(19) United States
(12) Patent Application Publication
(10) Pub. No.: US 2011/0110916 A1
(43) Pub. Date: May 12, 2011

(54) METHODS FOR TREATING AND/OR PREVENTING CARDIOMYOPATHIES BY ERK OR JNK INHIBITION

(76) Inventors: Howard J. Worman, New York, NY (US); Antoine Muchir, New York, NY (US)

(21) Appl. No.: 12/917,136

(22) Filed: Nov. 1, 2010

Related U.S. Application Data
(63) Continuation-in-part of application No. PCT/US09/42614, filed on May 1, 2009.
(60) Provisional application No. 61/049,462, filed on May 1, 2008.
(60) Provisional application No. 61/055,780, filed on May 23, 2008.

(51) Int. Cl. A61K 38/45; A61K 31/166; A61K 31/4184; A61K 31/352; A61K 31/277; A61K 31/416; A61K 31/454; A61P 9/00

(52) U.S. Cl. 424/94.5; 514/575; 514/394; 514/456; 514/523; 514/406; 514/326

(57) ABSTRACT

Provided is a method of treating or preventing a cardiomyopathy associated with activation of at least one kinase in the MAP kinase signaling pathway in heart tissue by providing to a subject an inhibitor of at least one kinase in the ERK signaling pathway or in the JNK signaling pathway, or both. In some embodiments, the cardiomyopathy is associated with one or more mutations in the LMNA gene, which encodes A-type nuclear lamins, or in the EMD gene, which encodes an inner nuclear membrane protein.

This application is a continuation-in-part of International Application No. PCT/US2009/42614 filed on May 1, 2009, which claims the benefit of priority of U.S. provisional applications Ser. No. 61/049,462, filed May 1, 2008, and Ser. No. 61/055,780, filed May 23, 2008. The disclosure of the aforementioned provisional applications, and of all patents, patent applications, and publications cited herein, are hereby incorporated by reference in their entirety.

The work described herein was supported in whole, or in part, by National Institutes of Health grant No. R01AR048997. Thus, the United States government has certain rights to the invention.

### BACKGROUND OF THE INVENTION

Cardiomyopathies may be caused a variety of factors, including environmental factors, genetic mutations, disruption of cell signaling pathways, and various other etiologies. The present invention is directed in part to methods of treating cardiomyopathies that are associated with activation of MAP kinase signaling pathways. Emery-Driefuss muscular dystrophy (EDMD) is characterized by genetic (inherited) cardiomyopathies. Acquired cardiomyopathies, such as hypertrophic cardiomyopathy, are also associated with MAP kinase activation (150).

Emery-Dreifuss muscular dystrophy (EDMD) results in cardiac disease, the initial presentation being atrioventricular conduction block followed by dilated cardiomyopathy (1). EDMD is also characterized by joint contractures in the spine, neck, elbows, and Achilles tendons, and progressive skeletal muscle weakness and wasting in a humero-peroneal distribution. EDMD was initially described as an X-linked inherited disorder, but it is now known that there are autosomal dominant and recessive forms of EDMD (100). X-linked EDMD is associated with mutations in the EMD gene (2, 4), while autosomal dominant and recessive EDMD is associated with mutations in the LMNA gene (5, 6).

The EMD gene encodes the ubiquitously expressed inner nuclear membrane protein emerin (3, 4). The LMNA gene encodes the widely expressed A-type nuclear lamins, of which lamin A and lamin C are the predominant somatic cell isoforms (8). Nuclear lamins are intermediate filament proteins that polymerize to form 10 nm diameter filaments on the inner aspect of the inner nuclear membrane (9-12). The lamina interacts with integral proteins in the inner nuclear membrane and provides anchorage sites for chromatin and structural support to the nuclear envelope (7). Many of the disease-causing A-type lamin mutants lead to disruption of the nuclear lamina and abnormal nuclear envelope architecture when expressed in cells (7).

In addition to playing a role in EDMD, mutations in the EMD and LMNA genes are associated with other cardiomyopathies, and indeed other non-cardiac diseases. For example, mutations in LMNA encoding A-type nuclear lamins cause several diverse diseases often referred to as laminopathies (7, 128), which, in addition to autosomal dominant and recessive EDMD, include dilated cardiomyopathy type 1A with conduction defect (68) and limb-girdle muscular dystrophy type 1B (69). These are a subset of the laminopathies that affect striated muscle (5, 6, 63, 39). A common feature of these disorders is cardiomyopathy. Indeed it is believed that 8% of familial and sporadic cardiomyopathies may be caused by mutations in the LMNA gene (129). While implantable pacemakers and defibrillators can prevent complications of cardiac dysrhythmias that occur early in these disorders, affected individuals eventually develop heart failure for which there is no curative treatment and cardiac transplantation is ultimately necessary (129-131). LMNA mutations are also associated with Charcot-Marie-Tooth disease type 2B1 (70) (a peripheral neuropathy with secondary muscle wasting and weakness), Dunnigan-type familial partial lipodystrophy (71-73) which affects adipose tissue (74), mandibuloacral dysplasia (75), Hutchison-Gilford progeria syndrome (76, 77), atypical Werner syndrome (78), neonatal lethal restrictive dermopathy (79), and disorders characterized by accelerated aging.

Despite that widespread expression of the EMD and LMNA genes, EDMD selectively affects striated muscle and tendons. Two main hypotheses have been proposed attempting to connect the pathophysiology of EDMD to functions of A-type lamins and emerin (7). The “mechanical stress” hypothesis proposes that the ability of A-type lamins and emerin to maintain the mechanical integrity of cells subject to stress is altered when LMNA or EMD genes are mutated. The “gene expression” hypothesis proposes a specific role of A-type lamins and emerin in proper tissue-selective gene expression. These hypotheses are not necessarily mutually exclusive, as altered nuclear mechanics and abnormal expression of stress-response genes have both been observed in cells lacking A-type lamins (13). However, despite data obtained mostly from cultured cells and in vitro binding assays that have lead to the “mechanical stress” and “gene expression” hypotheses, there are scant experimental results linking LMNA and EMD mutations to pathogenic pathways in affected tissues.

### SUMMARY OF THE INVENTION

We have determined the effects of an Lmna H222P mutation on signaling pathways involved in the development of cardiomyopathy in a knock-in mouse model of autosomal dominant Emery-Dreifuss muscular dystrophy. This is a model of inherited or genetic cardiomyopathy. Analysis of genome-wide expression profiles in hearts using Affymetrix GeneChips showed statistically significant differences in expression of genes in the MAPK pathways at the incipience of the development of clinical disease. Using real-time PCR, we showed that activation of MAPK pathways preceded clinical signs or detectable molecular markers of cardiomyopathy. In heart tissue and isolated cardiomyocytes, there was activation of MAPK cascades and downstream targets, implicated previously in the pathogenesis of cardiomyopathy. Expression of H222P lamin A in cultured cells activated MAPKs and downstream target genes. Activation of MAPK signaling by mutant A-type lamins could be a cornerstone in the development of heart disease in autosomal dominant Emery-Dreifuss muscular dystrophy.

We used the JNK inhibitor SP600125 (Calbiochem), which is a cell-permeable and selective inhibitor of all JNK isoforms (80-82), and PD98059 (Calbiochem), U0126 (EMD Biosciences), and MEK1/2 (EMD Biosciences), which are cell-permeable and selective for ERK isoforms (83-88). These compounds specifically block the MAP kinase kinases responsible for phosphorylating (activating) JNKs and ERKs. Lmna^{H222P/H222P} mice treated with MAPK inhibitors showed significantly improved ejection fraction and left ventricular end diastolic diameter as assessed by echocardiography, showing improvement in cardiac function. Kinase activation and activation of downstream genes were also inhibited in hearts of treated mice. Activation of ERK, JNK, or ERK plus JNK can lead to heart disease in Emery-Dreifuss muscular dystrophy and other cardiomyopathies. ERK, JNK, or ERK plus JNK inhibitors can block kinase activity and prevent onset of, improve or slow progression of, and/or improve cardiac function in cardiomyopathy in the Lmna^{H222P/H222P} mouse model of Emery-Dreifuss muscular dystrophy. Inhibitors to decrease activation can be used as treatment.

Therefore, this invention is based, in part, on the discovery that the JNK and ERK branches of the MAP kinase cascade are activated in mouse models of autosomal and X-linked EDMD, and the discovery that this activation occurs prior to the appearance of cardiac disease, suggesting that it is a primary pathogenic mechanism. The invention is also based, in part, on the discovery that, along with activation of JNK and ERK, EDMD is also associated with increased expression of “downstream” transcription factors, such as c-Jun, and genes they activate encoding sarcomeric proteins such as myosins and sacrolipin.

In one aspect, the invention provides a method of treating or preventing a cardiomyopathy associated with activation of at least one kinase in the mitogen-activated protein kinase (MAPK) signaling pathway in heart tissue, the method comprising providing to a subject an inhibitor of at least one kinase in the extracellular signal-regulated kinase (ERK) signaling pathway, or an inhibitor of at least one kinase in the c-Jun N-terminal kinase (INK) signaling pathway, or both.

In one embodiment, the cardiomyopathy is a genetic, or inherited, cardiomyopathy. For example, the cardiomyopathy can be associated with one or more mutations in LMNA or EMD. In another embodiment, the cardiomyopathy is an acquired cardimyopathy. In some embodiments, the cardiomyopathy can be a dilated cardiomyopathy or a hypertrophic cardiomyopathy.

The kinase in the ERK signaling pathway can be, for example, a MAPK/ERK kinase (MEK), in particular, MEK1 or MEK2.

The kinase in the JNK signaling pathwaycan be a JNK.

In one embodiment, the inhibitor of at least one kinase in the ERK signaling pathway is selected from the group consisting of a chromone and a flavone. The ERK signaling pathway inhibitor can be selected from the group consisting of 2-(2-amino-3-methoxyphenyl)-4H-1-benzopyran-4-one (PD98059), 1,4-diamino-2,3-dicyano-1,4-bis(2-aminophenylthio)butadiene (U0126), Z-& E-a-(amino-((4-aminophenyl)thio)methylene)-2-(trifluoromethyl)benzeneacetonitrile (MEK1/2), PD0325901, AZD6244/ARRY-142886, and ARRY-438162. In a preferable embodiment, the inhibitor of at least one kinase in the ERK signaling pathway is PD98059.

In a further embodiment, the inhibitor of at least one kinase in the JNK signaling pathway can be an anthrapyrazolone. In a preferred embodiment, the anthrapyrazolone is anthra[1,9-cd]pyrazol-6(2H)-one (SP600125). The inhibitor of at least one kinase in the JNK signaling pathway can be CC-401.

In one aspect, treating a cardiomyopathy comprises improving cardiac function or preventing deterioration in cardiac function. Improving cardiac function or preventing deterioration in cardiac function can comprise increasing at least one of ejection fraction or fractional shortening. Improving cardiac function or preventing deterioration in cardiac function can also comprise decreasing at least one of left ventricular end systolic diameter or left ventricular end diastolic diameter.

For purposes of the present invention, treating or preventing cardiomyopathy can comprise reducing expression of at least one molecular marker of cardiomyopathy. In one embodiment, the molecular marker is selected from the group consisting of atrial natriuretic factor, brain natriuretic factor, Bcl-2, Elk-1, c-Jun, JunD, Vegf, Myl7, Sln, and Elk 4. The molecular marker can be a sarcomere structure protein, for example, myosin.

The disclosure also provides a method for identification of a compound or a combination of compounds that is/are useful in the treatment of cardiac disease, such as cardiomyopathy, and/or improvement of cardiac function, the method comprising administering the compound or combination of compounds to an animal that is a model of cardiac disease or cardiac malfunction, wherein the model is a knock-in mouse model of autosomal dominant Emery-Dreifuss muscular dystrophy (Lmna^{H222P/H222P} mice), and determining whether the compound or combination of compounds improves cardiac function in the mouse, compared to a mouse model not so treated.

### BRIEF DESCRIPTION OF THE DRAWINGS

FIGS. 1A-1C show RNA expression profiling in hearts of Lmna H222P mice. (1A) Hierarchical clustering analysis of differentially expressed genes in hearts from Lmna^{+/+}, Lmna^{H222P/+} and Lmna^{H222P/H222P} mice. Rows indicate the expression of individual genes and vertical lines indicate each sample. For each gene, the ratio of transcript abundance in the samples to its abundance in the control is represented by color intensities (red indicates higher expression and green indicates lower expression). Transcriptional profiles of hearts from Lmna^{H222P/H222P} and Lmna^{H222P/+} mice show a greater degree of similarity to each other than to hearts from control Lmna^{+/+} mice. (1B) Volcano plots of absolute expression values (log₂[q-value]) determined by robust multichip analysis. For each probe set, expression in hearts from Lmna^{H222P/H222P} and Lmna^{H222P/+} mice is plotted. A two-fold threshold and q<0.05 was used to determine the probe sets significantly altered in the analysis (red dot squares). (1C) Validation of RNA expression profiling of selected genes in hearts from Lmna^{+/+}, Lmna^{H222P/+} and Lmna^{H222P/H222P} mice using real-time PCR. Bars indicate the fold overexpression of the indicated mRNA in hearts as calculated by the ΔΔC_{T} method. Values are means±standard deviations for n=6 samples per group. The real-time PCR were performed in triplicate with the different RNA samples. Matrices visualizing Affymetrix GeneChip data of corresponding probe sets of RNAs are shown at right of bar graph. In these matrices, each probe set is visualized as a row of colored squares with one square for each sample. Myh7, Myh4, Myl7, Acta2 and Sln show higher expression and Pttg lower expression compared to controls.

FIGS. 2A-2C show histological analysis of heart muscle in Lmna H222P mice and expression of myosins and ANF. (2A) Histological analysis of hearts from 10-week old control Lmna^{+/-} and Lmna^{H222P/H222P} mice. Representative fixed sections of left ventricles stained with hematoxylin and eosin (upper panels) and Gomori's trichrome (lower panels) are shown. Bars: 50 μm. Note normal-appearing cardiomyocytes and absence of fibrosis. (2B) Expression of myosins and ANF in hearts of 10-week old Lmna^{+/-}, Lmna^{H222P/+} and Lmna^{H222P/H222P} mice. Representative immunoblots for ANF, β-MHC and MLC-2 are shown. β-tubulin Ab labeling is shown as a loading control. (2C) Data in bar graphs are means±standard deviations of n=5 samples per group (*p<0.05).

FIGS. 3A-3B show MAPK signaling is activated in hearts and isolated cardiomyocytes from Lmna H222P mice. (3A) Detection of phosphorylated JNK and ERK1/2 in hearts and isolated cardiomyoctes from Lmna^{+/+}, Lmna^{H222P/+} and Lmna^{H222P/H222P} mice. JNK and ERK1/2 were measured by immunoblotting with Abs against total protein (JNK and ERK1/2) and phosphoprotein (pJNK and pERK1/2). Data in bar graphs are means±standard deviations of n=5 samples per group (*p<0.05, ***p<0.0005). (3B) Effect of MAPK activation on downstream targets in Lmna^{+/+}, Lmna^{H222P/+} and Lmna^{H222P/H222P} mice. Representative immunoblots using Abs that recognize phosphorylated c-Jun (pc-Jun), elk-1, bcl-2 and β-tubulin loading control are shown for proteins extracted from heart tissue and isolated ventricular cardiomyocytes.

FIGS. 4A-4C show immunofluorescence microscopic analysis of pERK1/2 in heart sections from Lmna^{H222P/H222P} mice. (4A) Sections of frozen heart from Lmna^{-/+} (top panel) and Lmna^{H222P/H222P} (bottom panel) mice were analyzed by immunofluorescence microscopy using Ab recognizing pERK1/2. Sections were counterstained with DAPI. Bars: 50 μm. (4B) Quantification of pERK1/2 labeling in cardiomyocytes from Lmna^{+/+} mice and Lmna^{H222P/H222P} mice. Cardiomyocytes are delimited by dotted line and intensity of emitted fluorescence is measured along the yellow line (a to b). Position of the nucleus and intensity of fluorescence using anti pERK1/2 Ab is shown in the diagram of a single cardiomyocyte. (4C) Bars indicate intensity of pERK1/2 fluorescence in the nucleus of the indicated hearts. Values are means±standard deviations for the intensity of nuclear fluorescence from n=90 cardiomyocytes from two different hearts per group (*p<0.05).

FIG. 5 shows expression of Elk-1, c-Jun, JunD and Elk-4 in various tissues from 10 week old Lmna+/+ and LmnaH222P/H222P mice. Summary of real-time PCR results in heart, skeletal muscle, lung, spleen and bladder are shown. Bars indicate the fold overexpression of the indicated mRNA normalized to Gapdh as calculated by the ΔΔCT method. Values are means±standard deviations for n=6 samples per group (*p<0.05, **p<0.005).

FIG. 6 shows time-course expression of genes activated by MAPK in hearts from LmnaH222P/H222P mice at 4, 7 and 10 weeks of age. Expression of Vegf, Myl7, Sln, c-Jun, Elk-1, JunD and Elk-4 in hearts of Lmna+/+ and LmnaH222P/H222P mice is shown. Bars indicate the fold overexpression of the indicated mRNA normalized to Gapdh as calculated by the ΔΔCT method. Values are means±standard deviations for n=6 samples per group (*p<0.05, **p<0.005).

FIGS. 7A-7F show Expression of H222P lamin A in transfected Cos-7 and C2C12 cells leads to increased phosphorylation and enhanced nuclear translocation of ERK1/2. (7A-7B) Effect of H222P lamin A expression on levels of pERK1/2 in transfected Cos-7 (A) and C2C12 (B) cells. Immunoblotting with pERK1/2 Ab or total ERK1/2 Ab was performed. Data are shown as means±standard deviations of n=11 (A) and n=7 (B) samples per group (*p<0.05). Significance of the results was determined using paired t-test (parametric) and a Wilcoxon test (non-parametric) Immunoblotting with GFP Ab are shown to demonstrate expression of proteins encoded by transfected plasmids Immunoblottings with β-actin Ab are shown as loading controls. (7C-7D) Effect of H222P lamin A on nuclear translocation of pERK1/2 in transfected Cos-7 (C) and C2C12 (D) cells. Representative photomicrographs are shown for non-transfected cells (NT), transfected cells expressing a GFP fusion of wild type lamin A (WT lamin A) and transfected cells expressing a GFP fusion of lamin A with the H222P amino acid substitution (H222P lamin A). Arrowheads show enhanced nuclear localization of pERK1/2 in cells expressing GFP-H222P lamin A Bars: 10 μm. (7E-7F) Percentages of Cos-7 (E) and C2C12 (F) cells with pERK1/2 primarily in the nucleus. Non-transfected cells (NT), transfected cells expressing a GFP fusion of wild type lamin A (WT lamin A) and transfected cells expressing a GFP fusion of lamin A with the H222P aa substitution (H222P lamin A) were randomly counted and scored for nuclear pERK1/2 (see arrowheads in C for example). Transfected cells were determined by presence of GFP signal. Values are means±standard deviations for n=200 cells per group (*p<0.05, **p<0.005). The person counting the cells was “blinded” as to which protein was expressed.

FIG. 8 shows activation of c-Jun and Elk-1 by expression of lamin A mutants. Cos-7 cells were transiently transfected with plasmids encoding wild type lamin A, lamin A with the indicated amino acid substitution and the associated phenotype to each mutation (e.g. EDMD or FPLD) or “empty vector” control. After 24h, luciferase activities induced by expression of c-Jun (upper panel) or Elk-1 (lower panel) were measured in cell lysates and normalized to β-gal activities obtained from a protein encoded by a co-transfected plasmid. Results are means±standard deviations of n=5 experiments (*p<0.05, **p<0.005).

FIG. 9 shows a model of how abnormalities of A-type lamins in the nuclear lamina may lead to cardiomyopathy. Abnormalities of A-type lamins in the nuclear lamina activates MAPK cascades, possibly via heterotrimeric G-protein receptors or by inducing stress responses by unknown mechanisms (?). This leads to enhanced phosphorylation of ERK and JNK1/2 and their subsequent nuclear translocation. In the nucleus, pERK1/2 and pJNK activate transcription factors such as elk-1, bcl-2, JunD, elk-4 and c-Jun, leading to increased synthesis of these proteins. Increased amounts and activities of transcription factors activated by pJNK and pERK1/2 alter expression of other genes, some encoding components of muscle fibers and sarcomeres. Aberrant expression of these proteins leads to development of cardiomyopathy.

FIGS. 10A-10D show expression of H222P lamin A in transfected Cos-7 and C2C12 leads to enhanced nuclear translocation of phospho-INK. (10A-10B) Effect of H222P lamin A on nuclear translocation of pJNK in transfected Cos-7 (A) and C2C12 (B) cells. Representative photomicrographs are shown for non-transfected cells (NT), transfected cells expressing a GFP fusion of wild type lamin A (WT lamin A) and transfected cells expressing a GFP fusion of lamin A with the H222P amino acid substitution (H222P lamin A). Arrowheads show enhanced nuclear localization of pJNK in cells expressing GFP-H222P lamin A Bars: 10 μm. (10C-10D) Percentages of Cos-7 (C) and C2C12 (D) cells with pJNK primarily in the nucleus. Non-transfected cells (NT), transfected cells expressing a GFP fusion of wild type lamin A (WT lamin A) and transfected cells expressing a GFP fusion of lamin A with the H222P aa substitution (H222P lamin A) were randomly counted and scored for nuclear pJNK (see arrowheads in A for example). Transfected cells were determined by presence of GFP signal. Values are means±standard deviations for n=200 cells per group (*p<0.05, **p<0.005).

FIG. 11 shows daily injection of inhibitors (PD98059, SP600125 or both altogether) in Lmna^{H222P/H222P} mice inhibits phosphorylation of their specific targets in heart from mice Immunoblots using anti-pERK1/2, anti-ERK1/2, anti-pJNK and anti-JNK antibodies on hearts from Lmna^{H222P/H222P} mice treated or not with the different inhibitors. Hearts from Lmna^{+/+} mice and Lmna^{H222P/H222P} mice treated with the vehicle alone (DMSO) were used as controls.

FIGS. 12A-12B show treatment of Lmna^{H222P/H222P} mice with MEK inhibitor PD98059 inhibits phosphorylation of ERK1/2 and activation of downstream target genes. (12A) Representative immunoblots using antibodies against phosphorylated ERK1/2 (pERK1/2) and antibodies against total ERK1/2 using proteins extracted from hearts from Lmna^{H222P/H222P} mice treated with PD98059 or placebo (DMSO). Results in hearts from Lmna^{+/+} mice and untreated Lmna^{H222P/H222P} mice are shown for comparison. Data in bar graphs are the quantification of phosphorylated ERK1/2 compared to total ERK1/2 measured by scanning the immunoblots and using Scion image Software (Scion Corporation). Values are means±standard deviations for n=3 samples from different animals per group. Results were compared using a two-tailed t test (*p<0.05). (12B) Quantitative real-time RT-PCR showing expression of RNAs of selected downstream target genes (Elk1, Elk4, Atf2, Atf4) of ERK signaling cascade in hearts from Lmna^{H222P/H222P} mice treated with PD98059 or placebo (DMSO). Results from hearts from Lmna^{+/-} mice and untreated Lmna^{H222P/H222P} mice are shown for comparison. Bars indicate the fold overexpression of the indicated mRNA in hearts. Values are means±standard deviations for n=4 samples from different animals per group. Reactions were performed in triplicate for each different RNA sample. Results were compared using a two-tailed t test (*p<0.05, **p<0.005).

FIGS. 13A-13B show the effect of MEK inhibitor PD98059 on cardiac expression of natriuretic peptides and myosins in Lmna^{H222P/H222P} mice. (13A) Immunoblot showing expression of natriuretic peptide precursor A (Nppa) in hearts from Lmna^{H222P/H222P} mice treated with PD98059 or placebo (DMSO). Results using hearts from Lmna^{+/-} mice and untreated Lmna^{H222P/H222P} mice are shown for comparison. Labeling with antibody against Gapdh is shown as a loading control. (13B) Quantitative real-time RT-PCR showing expression of RNAs from NppA and NppB genes, respectively encoding natriuretic peptide precursors A and B, and Myl4 and Myl7 genes, encoding myosin light chains, in hearts from Lmna^{H222P/H222P} mice treated with PD98059 or placebo (DMSO). Results from hearts from Lmna^{+/+} mice and untreated Lmna^{H222P/H222P} mice are shown for comparison. Bars indicate the fold overexpression of the indicated mRNA in hearts as calculated by the CT method. Values are means±standard deviations for n=4 samples from different animals per group. Reactions were performed in triplicate for each different RNA sample. Results were compared using a two-tailed t test (*p<0.05).

FIGS. 14A-14B show treatment with the MEK inhibitor PD98059 prevents dilation and deterioration of dynamics of the left ventricle in Lmna^{H222P/H222P} mice. (14A) Histological analysis of heart sections stained with hematoxylin and eosin from Lmna^{H222P/H222P} mice treated with PD98059 or placebo (DMSO). Hearts from Lmna^{+/+} mice and untreated Lmna^{H222P/H222P} mice are shown for comparison. The left ventricle is dilated in Lmna^{H222P/H222P} mice that were untreated or that received DMSO placebo whereas hearts from Lmna^{H222P/H222P} mice treated with PD98059 had a left ventricular chamber diameter is similar to Lmna^{+/+} mice. Scale bar: 1 mm. (14B) Transthoracic M-mode echocardiographic tracings in Lmna^{H222P/H222P} mice treated with PD98059 or placebo (DMSO). Tracings from Lmna^{+/+} mice and untreated Lmna^{H222P/H222P} mice are shown for comparison. Left ventricular end systolic diameter (LVESD) and left ventricular end diastolic diameter (LVEDD) are indicated. Note LVESD and LVEDD are similar in Lmna^{H222P/H222P} mice treated with PD98059 and decreased in Lmna^{H222P/H222P} mice that were untreated or that received DMSO placebo.

FIGS. 15A-15B show that treatment with PD98059 prevents abnormal elongation of cardiomyocyte nuclei in Lmna^{H222P/H222P} mice. (15A) Histological analysis of cross sections of hearts from Lmna^{H222P/H222P} mice treated with PD98059 or placebo (DMSO). Hearts from Lmna+/+ mice and untreated Lmna^{H222P/H222P} mice were used for comparisons. Sections are stained with hematoxylin and eosin. Inserts with yellow lines with arrowheads demonstrate measurement of nuclear length. Scale bar: 50 μm. (15B) Quantification of nuclear elongation in cardiomyocytes from mice. Cardiomyocyte nuclei are measured along the yellow lines with arrowheads as shown as examples in A. Bars indicate length of cardiomyocyte nuclei in the indicated hearts. Values are means±standard deviations for n=400 cardiomyocytes (*p<0.0005).

FIG. 16A is an immunoblot showing expression of total ERK1/2 and phosphorylated ERK1/2 (pERK1/2) in hearts from control and Lmna^{-/-} mice. Data in bar graphs are means±standard deviations derived from scanned immunoblots of n=4 samples per group (*p<0.05). FIG. 16B shows expression of mRNA encoded by c-Jun, Elk1, Mef2c, c-Fos, Atf2, JunD, Atf4 and Elk4 in hearts from control (open bars) and Lmna^{-/-} (dark bars) mice using real-time quantitative RT-PCR. Bars indicate fold overexpression of the indicated mRNA. Values are means±standard deviations for n=4 samples (*p<0.05, **p<0.005).

FIG. 17A shows expression of mRNA encoded by Gapdh, Emd and Lmna in HeLa cells transfected with siRNA duplexes against Gapdh, Emd and Lmna, using real-time quantitative RT-PCR. Bars indicate fold overexpression of the indicated mRNA. Values are means±standard deviations for n=4 samples (*p<0.05). FIG. 17B is an immunoblot showing expression of GAPDH, emerin and lamin A/C in HeLa cells transfected with siRNA duplexes against Gapdh, Emd and Lmna. Antibody against actin was used as a loading control. FIG. 17C shows expression of mRNA encoded by Gapdh, Emd and Lmna in C2C12 cells transfected with siRNA duplexes against Gapdh, Emd and Lmna, using real-time quantitative RT-PCR. Bars indicate fold overexpression of the indicated mRNA. Values are means±standard deviations for n=3 samples (*p<0.05). FIG. 17D is an immunoblot showing expression of GAPDH, emerin and lamin A/C in C2C12 cells transfected with siRNA duplexes against Gapdh, Emd and Lmna. Antibody against actin was used as a loading control.

FIG. 18A is a representative immunoblot showing expression of total ERK1/2 and phosphorylated ERK1/2 (pERK1/2) in HeLa cells transfected with siRNA duplexes against Gapdh, Lmna and Emd. FIG. 18B shows expression of downstream genes in ERK pathway in HeLa cells transfected with siRNA duplexes against Gapdh, Lmna and Emd. Real-time RT-PCR results for c-Jun, Elk1 and Elk4 are shown. Bars indicate the fold overexpression of the indicated mRNA normalized to Gapdh. Values are means±standard deviations for n=4 samples per group (*p<0.05). FIG. 18C is a representative immunoblot showing expression of total ERK1/2 and phosphorylated ERK1/2 (pERK1/2) in C2C12 cells transfected with siRNA duplexes against Gapdh, Lmna and Emd. FIG. 18D shows expression of downstream genes in ERK pathway in C2C12 cells transfected with siRNA duplexes against Gapdh, Lmna and Emd. Real-time RT-PCR results for c-Jun, Elk1 and Elk4 are shown. Bars indicate the fold overexpression of the indicated mRNA normalized to Gapdh. Values are means±standard deviations for n=4 samples per group (*p<0.05).

FIG. 19A shows the effect of siRNAs on nuclear translocation of pERK in transfected HeLa cells. Representative photomicrographs are shown for mock transfected cells, cells transfected with siRNA against Gapdh (siRNA Gapdh), Emd (siRNA Emd) and Lmna (siRNA Lmna). Arrowheads show enhanced nuclear localization of pERK in cells transfected with Emd and Lmna siRNAs. Bars: 10 μm. Bar graph shows percentages of HeLa cells with pERK primarily in the nucleus (see arrowheads for example). Values are means±standard deviations for n=200 cells per group (*p<0.05). FIG. 19B shows the effect of siRNAs on nuclear translocation of pERK in transfected C2C12 cells. Representative photomicrographs are shown for mock transfected cells, cells transfected with siRNA against Gapdh (siRNA Gapdh), Emd (siRNA Emd) and Lmna (siRNA Lmna). Arrowheads show enhanced nuclear localization of pERK in cells transfected with Emd and Lmna siRNAs. Bars: 10 μm. Bar graph shows percentages of C2C12 cells with pERK primarily in the nucleus (see arrowheads for example). Values are means±standard deviations for n=150 cells per group (*p<0.05).

FIG. 20A shows an immunoblot showing the effect of the MEK inhibitor PD98059 on the expression of total ERK1/2 and phosphorylated ERK1/2 in HeLa cells transfected with siRNAs against Gapdh, Lmna and Emd. FIG. 20B (upper part) is an immunoblot showing effect of the MEK inhibitor PD98059 on the expression of total ERK1/2 and phosphorylated ERK1/2 in C2C12 cells transfected with siRNAs against Gapdh, Lmna and Emd. Lower part shows results of ELISA showing effect of the MEK inhibitor PD98059 on the expression of total ERK1/2 and phosphorylated ERK1/2 in C2C12 cells transfected with siRNAs against Gapdh, Lmna and Emd. Bar graph shows the relative phosphorylation of ERK1/2. Values are means±standard deviations for n=3 samples per group (*p<0.05 when compared to mock treatment, #p<0.05 when compared C2C12 cells with or without addition of PD98059).

FIG. 21 shows representative immunoblots using antibodies against phosphorylated ERK1/2 (pERK1/2) and antibodies against total ERK1/2 using proteins extracted from hearts from Lmna^{H222P/H222P} mice treated with PD0325901 (n=4), AZD6244 (n=4), CI-1040 (n=4) or placebo (DMSO) (n=4). The graphs show quantification of pERK/total ERK for each group. Comparison between PD0325901-treated, AZD6244-treated and CI-1040-treated Lmna^{H222P/H222P} mice compared to DMSO-treated Lmna^{H222P/H222P} mice; **p<0.005.

FIG. 22 shows quantitative real-time RT-PCR showing expression of RNAs from NppA and NppB genes, respectively, encoding atrial natriuretic peptide and brain natriuretic in hearts from Lmna^{H222P/H222P} mice treated with PD0325901, AZD6244, CI-1040 or placebo (DMSO). Bars indicate the fold overexpression of the indicated mRNA in hearts as calculated by the CT method. Values are means±standard deviations for n=4 samples from different animals per group. Reactions were performed in triplicate for each different RNA sample. Results were compared using a Welch's ANOVA comparison (**p<0.005, ***p<0.0005, n.s=not significant).

FIG. 23 shows serum amount of atrial natriuretic factor in hearts from Lmna^{H222P/H222P} mice treated with PD0325901, AZD6244, CI-1040 or placebo (DMSO). Bars indicate the fold expression ratio (after treatment/before treatment) of the atrial natriuretic factor in hearts. Values are means±standard deviations for DMSO-treated (n=8), PD0325901-treated (n=6), AZD6244 (n=7) and CI-1040 (n=6) samples from different animals per group. (**p<0.005, n.s=not significant).

FIG. 24 shows quantitative real-time RT-PCR showing expression of RNAs from Mlc-1a and Mlc-2a genes, respectively, encoding myosin light chains, in hearts from Lmna^{H222P/H222P} mice treated with PD0325901, AZD6244, CI-1040 or placebo (DMSO). Bars indicate the fold overexpression of the indicated mRNA in hearts as calculated by the CT method. Values are means±standard deviations for n=4 samples from different animals per group. Reactions were performed in triplicate for each different RNA sample. Results were compared using a Welch's ANOVA comparison (**p<0.005, ***p<0.0005).

FIG. 25 shows echocardiographic measurement of LVEDD from Lmna^{H222P/H222P} mice treated with PD0325901 (n=11), AZD6244 (n=9), CI-1040 (n=6) or placebo (DMSO) (n=12). Results were compared using a Welch's ANOVA comparison (*p<0.05, ***p<0.0005).

FIG. 26 shows echocardiographic measurement of LVESD from Lmna^{H222P/H222P} mice treated with PD0325901 (n=11), AZD6244 (n=9), CI-1040 (n=6) or placebo (DMSO) (n=12). Results were compared using a Welch's ANOVA comparison (*p<0.05).

FIG. 27 shows enchocardiographic measurement of FS from Lmna^{H222P/H222P} mice treated with PD0325901 (n=11), AZD6244 (n=9), CI-1040 (n=6) or placebo (DMSO) (n=12).

FIG. 28A shows Gomori's trichrome staining of cross-sections of hearts from Lmna^{H222P/H222P} mice treated with PD0325901, AZD6244, CI-1040 or DMSO. Scale bar: 200 μm (upper panel) and 10 μm (lower panel). FIG. 28B shows quantification of fibrotic area in hearts from mice. Bars indicate the percentage of fibrosis per surface area of myocardium examined in hearts from Lmna^{H222P/H222P} mice treated with PD0325901 (n=5), AZD6244 (n=3), CI-1040 (n=2) or DMSO (n=4). *P<0.05. FIG. 28C shows the effect of MEK1/2 inhibitors on cardiac expression of genes encoding collagen in Lmna^{H222P/H222P} mice. Bar graphs indicate the expression of Colla1 and Colla2 in heart from Lmna^{H222P/H222P} mice treated with PD0325901, AZD6244, CI-1040 or DMSO. n=4 in each group. Values were obtained using the ΔΔCT method using Gapdh as housekeeping gene (see Full Materials and Methods). *P<0.05, **P<0.005, ***P<0.0005.

FIG. 29 shows the abnormal activation of ERK signaling in heart from patients with EDMD with cardiomyopathy. FIG. 29A shows representative immunoblots using antibodies against phosphorylated ERK1/2 (pERK1/2) and antibodies against total ERK1/2 using proteins extracted from hearts from patients and unaffected individuals. FIG. 29B shows quantification of pERK/total ERK for each individual.

FIG. 30 shows quantitative real-time RT-PCR showing expression of RNAs from PPARγ (FIG. 30A), NppA (FIG. 30B), and NppB genes (FIG. 30C), respectively, encoding atrial natriuretic peptide and brain natriuretic in hearts from PPARγ transgenic mice (model of lipotoxic cardiomyopathy). Bars indicate the fold overexpression of the indicated mRNA in hearts as calculated by the CT method. Values are means±standard deviations for n=4 samples from different animals per group. FIG. 30D shows expression of phosphoERK signaling in heart from PPARγ transgenic mice.

FIG. 31A shows representative immunoblots using antibodies against phophorylated ERK1/2 (p-ERK) and total ERK1/2 (ERK) and (FIG. 31B) against phophorylated JNK (p-JNK) and total JNK to probe proteins extracted from hearts from Lmna^{H222P/H222P} mice treated with PD98059, SP600125 or DMSO. Blots of proteins extracted from hearts of Lmna^{+/+} mice are shown for comparison. FIGS. 31C-D show quantification of (FIG. 31C) pERK/total ERK and (FIG. 31D) pJNK/total JNK. n=4 in each group. Comparison between DMSO-treated Lmna^{H222P/H222P} mice and Lmna^{+/+} mice; *P<0.05. Comparison between PD98059-treated and SP600125-treated and DMSO-treated Lmna^{H222P/H222P} mice; #P<0.05, ##P<0.005, n.s.: not significant.

FIG. 32 shows effect of PD98059 and SP600125 on cardiac expression of natriuretic peptides and myosin light chain in Lmna^{H222P/H222P} mice. Dot diagrams indicate the expression levels of Mlc-2a mRNA encoding the cardiac isoform of myosin light chain, Nppa mRNA encoding the atrial natriuretic factor and Nppb encoding the brain natriuretic peptide in hearts from Lmna^{+/+} mice and Lmna^{H222P/H222P} mice treated with PD98059, SP600125 or DMSO. n=4 in each group. Values were obtained using the ΔΔCT method using Gapdh as housekeeping gene (see Full Materials and Methods). *P<0.05, **P<0.005, #P<0.05, ##P<0.005.

FIG. 33 shows representative transthoracic M-mode echocardiographic tracings from Lmna^{H222P/H222P} mice treated with PD98059, SP600125 or DMSO. Tracings from Lmna^{+/+} mice are shown for comparison. LVESD and LVEDD are indicated.

FIG. 34A-B shows (FIG. 34A) Sirius red and (FIG. 34B) Gomori's trichrome staining of cross-sections of hearts from Lmna^{H222P/H222P} mice treated with PD98059, SP600125 or DMSO. A cross-section of heart from a Lmna^{+/+} mouse is shown for comparison. Scale bar: 50 μm. FIG. 34C shows quantification of fibrotic area in hearts from mice. n=3 in each group. Y-axis corresponds to the area (pixels) and X-axis represents the color spectrum (red corresponds to the muscle tissue and blue corresponds to the connective tissue). FIG. 34D is a graph showing the percentage of fibrosis per surface area of myocardium examined in hearts from Lmna^{+/+} mice and Lmna^{H222P/H222P} mice treated with PD98059, SP600125 or DMSO. n=3 in each group. ***P<0.0005, ###P<0.0005.

FIG. 35 show the effect of PD98059 and SP600125 on cardiac expression of genes encoding collagen and fibronectin in Lmna^{H222P/H222P} mice. Dot diagrams indicate the expression of Colla1, Colla2 and Fn1 in heart from Lmna^{+/+} mice and Lmna^{H222P/H222P} mice treated with PD98059, SP600125 or DMSO. n=3 in each group. Values were obtained using the ΔΔCT method using Gapdh as housekeeping gene (see Full Materials and Methods). *P<0.05, #P<0.05.

FIG. 36A shows a histological analysis of cross-sections of hearts from Lmna^{H222P/H222P} mice treated with PD98059, SP600125 or DMSO. Heart from a Lmna^{+/+} mouse is shown for comparison. Sections are stained with hematoxylin and eosin. Yellow lines with arrowheads demonstrate the measurement of nuclear length. Scale bar: 25 μm. FIG. 36B shows quantification of nuclear elongation in cardiomyocytes from mice. Cardiomyocyte nuclei were measured along the yellow lines with arrowheads. Bars indicate the length of cardiomyocyte nuclei in the indicated hearts. Values are means±SEM for n=150, 290, 690 and 575 cardiomyocytes from Lmna^{+/+} mice, DMSO-treated Lmna^{H222P/H222P} mice, PD98059-treated Lmna^{H222P/H222P} mice, and SP600125-treated Lmna^{H222P/H222P} mice, respectively. ***P<0.0005, ###P<0.0005.

FIG. 37A shows representative immunoblots using antibodies against phophorylated ERK1/2 (p-ERK) and total ERK1/2 (ERK) and against phophorylated JNK (p-JNK) and total JNK to probe proteins extracted from hearts from Lmna^{H222P/H222P} mice treated with PD98059, SP600125 or DMSO. FIG. 37B shows echocardiograhic data at 24 weeks for Lmna^{H222P/H222P} mice treated with DMSO, PD98059 or SP600125 from 19 to 24 weeks. Graphs show LVEDD, LVESD and FS for each treatment group. Values for each individual mouse and means±SEM are shown. *P<0.05, n.s.: not significant.

FIG. 38 shows effect of PD98059 and SP600125 on cardiac expression of natriuretic peptides, myosin light chain and collagen I in Lmna^{H222P/H222P} mice. Dot diagrams indicate the expression levels of mRNAs in hearts from Lmna^{H222P/H222P} mice treated with PD98059 (n=3), SP600125 (n=3) or DMSO (n=4). Values were obtained using the ΔΔCT method using Gapdh as housekeeping gene (see Methods). *P<0.05, n.s.: not significant that ERK1/2 are activated in hearts from human patients with ischemic heart and obstructive cardiomyopathy.

FIG. 39 is a graph showing the effect of Ekr1 gene depletion on cardiac fractional shortening (FS) in Lmna H222P mice at 16 weeks of age. Dot diagrams indicate FS for wild type mice with two normal copies of the Lmna and Erk1 genes (WT 16w), LmnaH222P/H222P mice with both copies of the Erk1 gene present (ERK+/+ Lmna H222P/H222P), mice with both Erk1 genes deleted but two wild type Lmna alleles (ERK −/− Lmna+/+), LmnaH222P/H222P mice with one copy of the Erk1 gene deleted (ERK +/− Lmna H222P/H222P) and LmnaH222P/H222P mice with both copies of the Erk1 gene deleted (ERK −/− Lmna H222P/H222P). Deletion of two copies of the Erk1 gene significantly improves FS in Lmna H222P/H222P mice. near tho n.s.=not significant; ***p<<0.0005

FIG. 40 is a photographic image of a western blot showing ERK activation in human hearts. ERK1/2 are activated in heart from patients with ischemic heart and obstructive cardiomyopathy.

### DETAILED DESCRIPTION OF THE INVENTION

### MAP Kinase Signaling

Mitogen-activated protein (MAP) kinases are serine/threonine-specific protein kinases that respond to extracellular stimuli (mitogens). MAP kinases are successively acting phosphorylases that function as regulators of cell growth, differentiation and transformation and have been implicated in many physiological and pathological processes (22, 28, 29). MAP kinase signaling cascades have been evolutionarily well-conserved from yeast to mammals. There are several types of MAP kinases, including, but not limited to the “extracellular signal-regulated kinases” or “ERKS” (such as ERK1 and ERK2), and the “c-jun N-terminal kinases” or “JNKs” (such as MAPK8, MAPK9, and MAPK10). Activation of the ERK subfamily of MAPKs is generally mediated by receptor protein tyrosine kinases or G-protein-coupled receptors (41). The JNK subfamily of MAPKs are generally activated by factors such as osmotic stress (42) and physical stress (43).

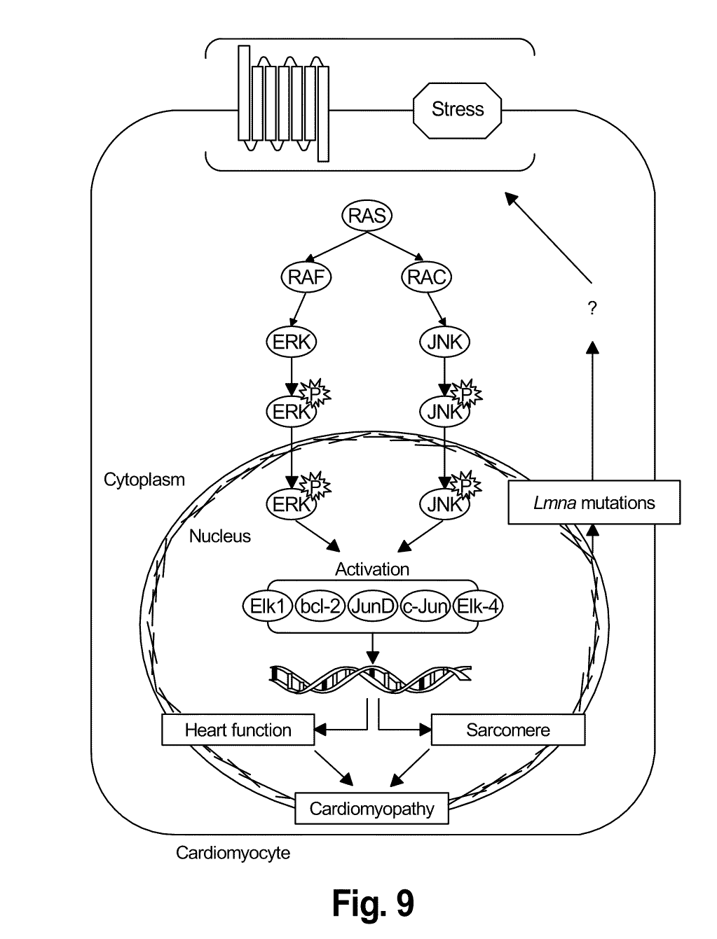

Several downstream target genes are activated by MAPKs including, but not limited to, Elk-1, Bcl-2, JunD, Elk-4 and c-Jun. Activation of these targets can in turn regulate expression of additional genes, including those encoding proteins involved in sarcomere structure, cardiomyofiber organization and other aspects of heart function (30, 31). Abnormal expression of these proteins can lead to cardiomyopathy (See FIG. 9). Examples of proteins in the ERK signaling pathway are Raf-1 and MAPK/ERK kinases (MEK). Examples of proteins in the JNK signaling pathway are c-Jun, JNK kinase 1, JNK kinase 2, and JNK Interacting Proteins.

MAP kinase signaling pathways, such as the JNK and ERK type signaling pathways, are well known to those of skill in the art. Such pathways are described in, for example, Maosong & Elion (151), Chang & Karin (152), Chen et al. (153), Pearson et al. (154), Davis et al. (155), Roux & Blenis (156), and the web site of Cell Signaling.com, the contents of each of which are hereby incorporated by reference.

### Inhibitors

The present invention provides methods for the treatment and/or prevention of cardiomyopathies which comprise administration of one or more inhibitors. The inhibitors of the invention include inhibitors of kinases in the extracellular signal-regulated kinase or “ERK” signaling pathway(s), and inhibitors of kinases in the c-jun N-terminal kinase or “JNK” signaling pathway(s). Any suitable inhibitor of a kinase in the ERK and/or JNK pathways may be used. Such inhibitors may be, for example, small molecule drugs, peptide agents, peptidomimetic agents, antibodies, inhibitory RNA molecules and the like. One of skill in the art will understand that these and other types of agents may be used to inhibit kinases in the ERK and/or JNK pathways.

In one embodiment, an inhibitor of the invention is a small molecule inhibitor of a kinase in an ERK signaling pathway. Such inhibitors include, but are not limited to, chromone and flavone type inhibitors. Other suitable small molecule inhibitors or ERK pathway kinases include, but are not limited to, 2-(2-amino-3-methoxyphenyl)-4H-1-benzopyran-4-one (PD98059) (see reference 168), PD0325901 (Pfizer), AZD6244/ARRY-142886 (AstraZeneca/Array BioPharma), ARRY-438162 (Array BioPharma), PD198306, PD0325901 (reference 172), AZD8330 (reference 172), CI-1040, PD184161, Z-& E-a-(Amino-((4-aminophenyl)thio)methylene)-2-(trifluoromethyl)benzeneacetonitrile (SL327) (see references 157-163), 1,4-Diamino-2,3-dicyano-1,4-bis(2-aminophenylthio)butadiene (see reference 164-166), U0126 (see reference 167 and 168), GW 5074 (reference 168), BAY 43-9006 (reference 168), PD184352 (reference 168), Wyeth-Ayerst Compound 14 (reference 168), Ro 09-2210 (reference 168), L-783.277 (reference 168), FR180204 (reference 169), 3-(2-aminoethyl)-5-))4-ethoxyphenyl)methylene)-2,4-thiazolidinedione (PKI-ERK-005) (references 170, 171), CAY10561 (CAS 933786-58-4; Cayman Chemical), GSK1120212 (reference 172), RDEA119 (Ardea Biosciences; reference 172), XL518 (reference 172), and ARRY-704 (AstraZeneca).

In another embodiment, an inhibitor of the invention is a small molecule inhibitor of a kinase in a JNK signaling pathway. Such inhibitors include, but are not limited to, anthrapyrazolone type inhibitors. Other suitable small molecule inhibitors of JNK pathway kinases include, but are not limited to, anthra[1,9-cd]pyrazol-6(2H)-one (SP600125), CC-401 (Celgene), CEP-1347 (Cephalon), BI-78D3 (reference 173), and AS601245 (reference 175). U.S. Pat. No. 7,199,124 to Ohkawa et al. also describes JNK inhibitors suitable for use in this invention.

In other embodiments, the inhibitors of the invention are peptide or peptidomimetic inhibitors of a kinase in the ERK or JNK signaling pathways. Such inhibitors include, but are not limited to a peptide corresponding to the amino-terminal 13 amino acids of MEK1 (MPKKKPTPIQLNP [SEQ ID NO: 1]) (see reference 168) and the JNK inhibitor XG-102, TAT-coupled dextrogyre peptide (reference 174).

In yet other embodiments, the inhibitors of the invention are antibody inhibitors of a kinase in the ERK or JNK signaling pathways. Such inhibitors include, but are not limited to humanized antibodies, fully human antibodies, and antibody fragments that bind to and inhibit the function of a kinase in the ERK or JNK signaling pathways.

In yet other embodiments, the inhibitors of the invention are nucleotide-based inhibitors of a kinase in the ERK or JNK signaling pathways. Such inhibitors include, but are not limited to siRNAs, shRNAs, dsRNAs, microRNAs, antisense RNA molecules, and ribozymes, that inhibit the expression or activity of a kinase in the ERK or JNK signaling pathways. Such nucleotide-based inhibitors may comprise ribonucleotides, deoxyribonucleotides, or various artificial nucleotide derivatives.

One of skill in the art will understand that other agents may be useful as inhibitors of kinases in the ERK and/or JNK signaling pathways and may be used in conjunction with the methods of the invention.

### Administration

The inhibitors of the invention may be formulated into compositions for administration to subjects for the treatment and/or prevention of cardiomyopathies. Such compositions may comprise the inhibitors of the invention in admixture with one or more pharmaceutically acceptable diluents and/or carriers and optionally one or more other pharmaceutically acceptable additives. The pharmaceutically-acceptable diluents and/or carriers and any other additives must be “acceptable” in the sense of being compatible with the other ingredients of the composition and not deleterious to the subject to whom the composition will be administered. One of skill in the art can readily formulate the inhibitors of the invention into compositions suitable for administration to subjects, such as human subjects, for example using the teaching a standard text such as Remington's Pharmaceutical Sciences, 18th ed, (Mack Publishing Company: Easton, Pa., 1990), pp. 1635-36), and by taking into account the selected route of delivery.

Examples of diluents and/or carriers and/or other additives that may be used include, but are not limited to, water, glycols, oils, alcohols, aqueous solvents, organic solvents, DMSO, saline solutions, physiological buffer solutions, peptide carriers, starches, sugars, preservatives, antioxidants, coloring agents, pH buffering agents, granulating agents, lubricants, binders, disintegrating agents, emulsifiers, binders, excipients, extenders, glidants, solubilizers, stabilizers, surface active agents, suspending agents, tonicity agents, viscosity-altering agents, carboxymethyl cellulose, crystalline cellulose, glycerin, gum arabic, lactose, magnesium stearate, methyl cellulose, powders, saline, sodium alginate. The combination of diluents and/or carriers and/or other additives used can be varied taking into account the nature of the active agents used (for example the solubility and stability of the active agents), the route of delivery (e.g. oral, parenteral, etc.), whether the agents are to be delivered over an extended period (such as from a controlled-release capsule), whether the agents are to be co-administered with other agents, and various other factors. One of skill in the art will readily be able to formulate the compounds for the desired use without undue experimentation.

The inhibitors of the invention may be administered to a subject in an amount effective to treat or prevent a cardiomyopathy. One of skill in the art can readily determine what would be an effective amount of the inhibitors of the invention to be administered to a subject, taking into account whether the inhibitor is being used prophylactically or therapeutically, and taking into account other factors such as the age, weight and sex of the subject, any other drugs that the subject may be taking, any allergies or contraindications that the subject may have, and the like. For example, an effective amount can be determined by the skilled artisan using known procedures, including analysis of titration curves established in vitro or in vivo. Also, one of skill in the art can determine the effective dose from performing pilot experiments in suitable animal model species and scaling the doses up or down depending on the subjects weight etc. Effective amounts can also be determined by performing clinical trials in individuals of the same species as the subject, for example starting at a low dose and gradually increasing the dose and monitoring the effects on cardiopmyopathy. Appropriate dosing regimens can also be determined by one of skill in the art without undue experimentation, in order to determine, for example, whether to administer the agent in one single dose or in multiple doses, and in the case of multiple doses, to determine an effective interval between doses.

The inhibitors of the invention may be administered to a subject by any suitable method that allows the agent to exert its effect on the subject in vivo. For example, the compositions may be administered to the subject by known procedures including, but not limited to, by oral administration, sublingual or buccal administration, parenteral administration, transdermal administration, via inhalation, via nasal delivery, vaginally, rectally, and intramuscularly. The compounds of the invention may be administered parenterally, or by epifascial, intracapsular, intracutaneous, subcutaneous, intradermal, intrathecal, intramuscular, intraperitoneal, intrasternal, intravascular, intravenous, parenchymatous, or sublingual delivery. Delivery may be by injection, infusion, catheter delivery, or some other means, such as by tablet or spray. In one embodiment, the inhibitors of the invention are administered to the subject by way of delivery directly to the heart tissue, such as by way of a catheter inserted into, or in the proximity of the subject's heart, or by using delivery vehicles capable of targeting the drug to the heart. For example, the inhibitors of the invention may be conjugated to or administered in conjunction with an agent that is targeted to the heart, such as an antibody or antibody fragment.

For oral administration, a formulation of the inhibitors of the invention may be presented as capsules, tablets, powders, granules, or as a suspension or solution. The formulation may contain conventional additives, such as lactose, mannitol, cornstarch or potato starch, binders, crystalline cellulose, cellulose derivatives, acacia, cornstarch, gelatins, disintegrators, potato starch, sodium carboxymethylcellulose, dibasic calcium phosphate, anhydrous or sodium starch glycolate, lubricants, and/or or magnesium stearate.

For parenteral administration (i.e., administration by through a route other than the alimentary canal), the inhibitors of the invention may be combined with a sterile aqueous solution that is isotonic with the blood of the subject. Such a formulation may be prepared by dissolving the active ingredient in water containing physiologically-compatible substances, such as sodium chloride, glycine and the like, and having a buffered pH compatible with physiological conditions, so as to produce an aqueous solution, then rendering the solution sterile. The formulation may be presented in unit or multi-dose containers, such as sealed ampoules or vials. The formulation may be delivered by injection, infusion, or other means known in the art.

For transdermal administration, the inhibitors of the invention may be combined with skin penetration enhancers, such as propylene glycol, polyethylene glycol, isopropanol, ethanol, oleic acid, N-methylpyrrolidone and the like, which increase the permeability of the skin to the compounds of the invention and permit the compounds to penetrate through the skin and into the bloodstream. The inhibitors of the invention also may be further combined with a polymeric substance, such as ethylcellulose, hydroxypropyl cellulose, ethylene/vinylacetate, polyvinyl pyrrolidone, and the like, to provide the composition in gel form, which are dissolved in a solvent, such as methylene chloride, evaporated to the desired viscosity and then applied to backing material to provide a patch.

In some embodiments, the inhibitors of the invention are provided in unit dose form such as a tablet, capsule or single-dose injection or infusion vial.

In certain embodiments, the inhibitors of the invention may be used in combination with other agents useful for the treatment of cardiomyopathies. For example, in one embodiment, the inhibitors of the invention may be delivered to a subject as part of a composition containing one or more additional active agents. In another embodiment, the inhibitors of the invention may be delivered to a subject in a composition or formulation containing only that active agent, while one or more other agents useful for the treatment of a cardiomyopathy may be also be administered to the subject in one or more separate compositions or formulations.

The inhibitors of the invention and the other agents useful for the treatment of cardiomyopathies may be administered to the subject at the same time, or at different times. For example, the inhibitors of the invention and the other agents may be administered within minutes, hours, days, weeks, or months of each other, for example as part of the overall treatment regimen of a subject. The inhibitors of the invention may also be used in combination with surgical or other interventional treatment regimens used for the treatment of cardiomyopathies.

### Mouse Model of Cardiomyopathy

The present invention is a method of treating or preventing a MAPK-associated cardiomyopathy. A “MAPK-associated cardiomyopathy” is a cardiomyopathy that is characterized by activation of the MAPK signaling pathway in heart tissue. Cardiomyopathies can also be associated with activation of one or more members of the ERK signaling pathway. Cardiomyopathies can additionally be associated with activation of one or more members of the JNK signaling pathway.

The cardiomyopathy can be inherited, as in EDMD, or acquired. A cardiomyopathy that results from activation of MAPK signaling, particularly from activation of ERK signaling and/or activation of JNK signaling, can be treated or prevented by administration of an inhibitor of the ERK or JNK signaling pathways, regardless of whether the cardiomyopathy is inherited or acquired. The methods of the present invention are useful in the treatment of various types of cardiomyopathies, including dilated cardiomyopathy and hypertrophic cardiomyopathy.

It has not been known how certain mutations in LMNA encoding A-type lamins cause striated muscle disease. Therefore, it has been impossible to develop targeted treatments. To obtain information on the pathogenic abnormalities in cardiac tissue that may cause cardiomyopathy in autosomal dominant EDMD, we carried out a genome-wide RNA expression analysis in hearts from LmnaH222P/+ and LmnaH222P/H222P “knock in” mice, which serve as a model for the human disease. A detailed description of these mice has been published previously (14). In brief, male LmnaH222P/H222P mice develop cardiac chamber dilation, decreased left ventricle fractional shortening and hypokinesis detectable by echocardiography starting at 8 weeks of age. At 12 weeks of age, abnormalities of the conduction system become pronounced and are characterized primarily by an increased PR interval on electrocardiograms. Histological analysis shows pronounced left ventricular fibrosis and fiber degeneration by 16 weeks of age along with obvious atrial dilation. The male mice die between 4 and 9 months of age. In female mice, disease develops more slowly. Both male and female mice also develop problems with locomotion secondary to skeletal muscle myopathy.

We have selected to study cardiac tissue and function rather than skeletal muscle, the most significant reason being that cardiomyopathy is the life-threatening problem in human patients with EDMD. Furthermore, cardiac tissue is homogenous and therefore easier to study biochemically than skeletal muscle, with is regionally and variably affected in EDMD as well as mouse models of the disease. Cardiac function is also easier to assess in LmnaH222P/H222P mice than skeletal muscle function. For example, left ventricular contraction can be readily measured by echocardiography and, as cardiac dysfunction is the cause of early death in these mice, survival can be easily assessed.

### Linkage of LMNA Mutations to EDMD

Cellular mechanisms linking mutations in LMNA to cardiomyopathy are unknown. While several investigators have hypothesized that LMNA mutations lead to alterations in gene expression that could have tissue-selective pathogenic consequences (7), altered expression of functional groups of genes or activation of signal transduction pathways that can explain the development of disease have not been demonstrated in affected tissues. We addressed this issue by using genome-wide profiling in hearts from a mouse model of autosomal dominant EDMD. Our analysis lead us to focus on MAPK signaling because in a genome-wide expression analysis several genes related to this pathway had significantly altered expression in hearts of mice with the Lmna H222P mutation prior to development of significant cardiomyopathy.

In hearts of Lmna^{H222P/H222P} mice, we found significantly increased expression of transcripts encoding several downstream components of MAPK cascades, such as c-Jun and Elk-1, only by using real-time PCR. Increased expression of these transcripts, which was approximately 2-fold, was not detected in our microarray analysis. Similar discrepancies between microarrays and real-time PCR have been reported (26, 27), especially when the absolute expression levels are low or when the differences between experimental and control are relatively small, which was the case for the transcripts we measured. In addition to increased expression of transcripts encoding several components, activation of MAPK cascades in hearts of Lmna H222P mice was also strongly supported by increased levels of selected encoded proteins, increases in nuclear pERK1/2 and activation of ERK1/2 and JNK in cells transfected with constructs encoding Lamin A with aa substitutions causing EDMD.

MAPK activation occurred prior to significant cardiomyopathy in Lmna^{H222P/H222P} mice and also in Lmna^{H222P/+} mice, which do not develop clinical heart disease until 2 years of age. This is consistent with activation of MAPK signaling underlying development of disease rather than occurring as a consequence. The temporal differences to develop cardiomyopathy between heterozygous and homozygous mice may be a result of “dosage”, as JNK activation and increased expression of its downstream targets bcl-2, and phosphorylated c-Jun, appeared to be more significant in hearts from Lmna^{H222P/H222P} mice compared to hearts from Lmna^{H222P/+} mice. Several genes were also activated or repressed in heterozygous mice compared to homozygous mice; however, how this is related to development of disease remains to be investigated.

Results from previous studies have implicated activation of MAPKs in development of cardiomyopathy. Petrich et al. (19, 20) generated transgenic mice expressing an activated mutant of MKK7, a kinase activating JNK, specifically in heart. These mice developed dilated cardiomyopathy. Similar results have been observed in transgenic mice overexpressing mutants of MKK3 and MKK6, kinases that also activate MAPKs (32). Nicol et al. (33) generated transgenic mice over expressing MEKS, which activates ERK, in hearts and these mice developed dilated cardiomyopathy. JNK is also activated in dilated human hearts (34, 35). Recently, Rodriguez-Viciana et al. demonstrated that mutations in MEK1 and MEK2, which encode kinases that activate ERK1 and ERK2, cause cardio-facial-cutaneous syndrome in humans (36). The MEK mutants were more active than wild type in phosphorylating ERK. Transgenic mice expressing activated MEK1 similarly have enhanced ERK1/2 signaling and develop cardiomyopathy (37). Activation of the ERK cascade has also been reported in caveolin-3 (38), caveolin-1 (39) and p85 subunit of class I(A) PI3K (40) knockout mice, all of which develop cardiomyopathy at 2 months of age.

While it remains unclear how A-type lamins with aa substitutions activate MAPKs, our results show that they do so when expressed in transfected cells. Activation of the ERK subfamily of MAPKs is mediated by receptor protein tyrosine kinases or G-protein-coupled receptors (41). JNK subfamily of MAPK is activated by osmotic stress (42) and physical stress (43). It is possible that abnormalities in the nuclear lamina lead to activation of G-protein coupled or other receptors via an unknown mechanism (FIG. 9). Several investigators have hypothesized that alterations in response to stress may underlie the development of striated muscle diseases caused by LMNA mutations (7). Abnormal responses to stress in cells with abnormalities in A-type lamins could therefore impact on activation of JNK (FIG. 9). Fibroblasts from mice lacking A-type lamins have increased nuclear deformation and impaired viability under mechanical strain as well as attenuated NF-κB-regulated transcription in response to stress (14). In addition, we observed that expression of H222P lamin A in transfected cells lead to enhanced nuclear translocation of activated ERK and JNK. Smith et al. (44) have demonstrated that suppression of cell proliferation after retinoic acid-induced endoderm differentiation of embryonic stem and carcinoma cells is achieved by restricting nuclear entry of activated MAPK and an intact cytoskeleton is required for the restraint. Hence, interactions between the nuclear lamina and cytoskeletal components could influence nuclear translocation of activated MAPKs, with abnormalities in the lamina enhancing their nuclear localization. Recently, Ivorra et al. (45) highlighted a direct interaction between A-type lamins and the transcription factor c-fos. This raises the possibility that A-type lamins may bind to component of MAPK cascades and that the H222P aa substitution may alter such an interaction.

Our results provide a foundation upon which pharmacological interventions for treatment or prevention of cardiomyopathy in EDMD can be based. If mutant A-type lamins activate JNK and ERK, which in turn lead to gene expression alterations responsible for the development of cardiomyopathy, MAPK inhibitors could be used to treat or prevent disease. MAPK inhibitors have been studied as therapeutic agents for a wide range of diseases. JNK inhibitors have been shown to be beneficial in reducing myocardial ischemic injury (59), stroke (60), hearing impairment (61) and various neurodegenerative disorders (62). The availability of MAPK inhibitors with in vivo activities makes “clinical trials” to prevent or treat cardiomyopathy in Lmna^{H222P/H222P} mice possible. In addition, knock-out mouse models of ERK1/2 and JNK have been generated (63). Crossing those mice with Lmna^{H222P/H222P} mice could also establish if abolishing function of MAPKs can rescue cardiomyopathy.

Analysis of genome-wide expression changes in hearts from Lmna H222P mice revealed significant alterations in expression of genes involved in inflammation and fibrosis prior to detectable abnormalities in hearts examined using conventional histological methods. Ultimately, fibrosis with minimal inflammation occurs in hearts from Lmna^{H222P/H222P} mice (14) as well as Lmna^{N195K/N195K} (mice, another model of EDMD (64). This suggests that in addition to treatment with MAPK inhibitors, early treatment with anti-inflammatory or anti-fibrotic agents may benefit human subjects with EDMD.

### Prevention and Treatment of MAPK-Associated Cardiomyopathies

“Treating” cardiomyopathy includes the improvement of cardiac function in a patient with cardiomyopathy, as measured by (1) an increase in ejection fraction (EF), and/or (2) an increase in fractional shortening (FS), and/or (3) a decrease in left ventricular end systolic diameter (LVESD), and/or (4) a decrease in left ventricular end diastolic diameter (LVEDD). “Treating” cardiomyopathy additionally includes the prevention of further deterioration of cardiac function, as measured by the above parameters.

“Preventing” cardiomyopathy includes arresting the onset of physiological and/or molecular indications of cardiomyopathy. Physiological indicators of cardiomyopathy include: (1) decreased ejection fraction (EF), and/or (2) decreased fractional shortening (FS), and/or (3) increased left ventricular end systolic diameter (LVESD), and/or (4) increased left ventricular end diastolic diameter (LVEDD). Molecular indicators of cardiomyopathy include increased expression of certain markers, including, but not limited to: sarcomere structure proteins (including β-myosin heavy chain and myosin light chain 2), atrial natriuretic factor, brain natriuretic factor, phosphorylated JNK, phosphorylated ERK1/2, Bcl-2, Elk-1, phosphorylated c-Jun, JunD, Vegf, Myl7, Sln, and Elk 4.

Our work demonstrates that ERK and JNK inhibitors improve the cardiac phenotype in a mouse model of EDMD. Our work showed that the cardiac function was in part or totally recovered, following 8 weeks treatment using PD98059 and/or SP600125 or U0126 or MEK1/2. In Example 2, we administered the inhibitors before the appearance of cardiac symptoms in Lmna^{H222P/H222P} mice. In Example 3, we show that the inhibitors can also be administered when the cardiomyopathy is evident in Lmna^{H222P/H222P} mice (after 12 weeks), to demonstrate that the inhibitors can also reverse the existing cardiac phenotype.

PD98059 shows high specificity for MEK over other serine/threonine kinases (83, 136). However, it also has activity against cyclooxygenase-1 and cyclooxygenase-2 (137). It is therefore possible that the beneficial effects of PD98059 in Lmna^{H222P/H222P} mice could in part be due to cyclooxygenase inhibition. We do not however consider cyclooxygenase inhibition to be a major mechanism of action given the widespread use of non-steroidal anti-inflammatory drugs in clinical practice and absence of data showing any utility in preventing heart failure. In fact, retrospective populations cohort studies suggest that use of both cyclooxygenase-2 inhibitors and non-selective cyclooxygenase inhibitors are associated with exacerbation of heart failure in humans (138, 139). Nonetheless, future controlled experimental testing of cyclooxygenase inhibition in Lmna^{H222P/H222P} mice would be useful in determining if it also has any beneficial effect in delaying or preventing cardiomyopathy.

Similar to Lmna^{H222P/H222P} mice, we have shown abnormal activation of ERK signaling in hearts of Emd^{-/y} mice lacking the integral inner nuclear membrane protein emerin that binds to A-type lamins (69). In humans, EMD mutations resulting in lack of or reduced emerin in the nuclear envelope cause X-linked Emery-Dreifuss muscular dystrophy (2, 4, 140). Like the autosomally inherited form of the disease caused by LMNA mutations, dilated cardiomyopathy is a major feature of X-linked Emery-Dreifuss muscular dystrophy. Therefore, the present results in Lmna^{H222P/H222P} mice are likely to be relevant to cardiomyopathy caused by emerin deficiency. However, because the clinical phenotype of first-degree heart block in Emd^{-/y} mice greater than 40 weeks of age is very subtle and not readily measurable without intensive electrophysiologically monitoring (106, 107), we have deferred a trial of an ERK inhibitor in this animal model.

Our results provide initial proof of principle for ERK and/or JNK inhibition as a therapeutic option to prevent or delay the onset of heart failure in cardiomyopathy caused by LMNA mutation. The only other demonstration of improving an abnormal phenotype caused by mutations in the gene encoding A-type lamins in mammals is the use of a protein farnesyltransferase inhibitor to block prenylation of truncated prelamin A in mice carrying a mutation that causes Hutchinson-Gilford progeria syndrome (141, 142). In the present invention, treatment with a MEK inhibitor at an age when Lmna^{H222P/H222P} mice first begin to develop cardiac abnormalities maintained LV function at normal levels while untreated mice had approximately a 30% reduction in ejection fraction over a time period of 8 weeks. (See Example 2.)

In humans, the progression of cardiomyopathy caused by LMNA mutations in often rapid compared to other primary cardiomyopathies (129). Therefore, pharmacological interventions to slow progression should be clinically beneficial. Further preclinical investigation, including for example an analysis of effects on different tissues, skeletal myopathy and overall activity, will determine the safety and efficacy of ERK or JNK inhibition as a therapeutic intervention for dilated cardiomyopathy. It is worth noting that oral MEK inhibitors have already been safely administered to humans (99, 143). In sum, for treatment of cardiomyopathy, for example in EDMD patients, it appears important to identify a MAPK inhibitor that inhibits specifically the ERK branch or the JNK branch, which inhibitor is tolerated over the long-term.

### Activation of ERK Signaling by Reduced Expression of A-Type Lamins and Emerin

Our studies have shown that abnormalities in A-type lamins and emerin activated MAP kinases in the hearts of mouse models of X-linked and autosomal EDMD [69, 89]. We have analyzed affected and unaffected tissues in Lmna^{H222P/H222P} mice and found abnormal activation of genes downstream of ERK only in cardiac and to a more limited extent skeletal muscle [89]. We have similarly demonstrated abnormal activation of ERK and downstream genes in hearts of emerin-deficient mice [69]. Although the exact mechanism of activation remains unclear, these findings provide the basis for pharmacological therapies that can prevent or improve cardiac function in cardiomyopathies, such as those associated with EDMD. In the present invention, we describe activation of ERK in a third mouse model of EDMD and established a cellular model of activation of induced by siRNA-mediated knockdown of emerin and A-type lamins. (See Example 4.) We show that loss of A-type lamins in mouse heart and partial loss of A-type lamins and emerin in cultured cells leads to activation.

Loss of emerin leads to EDMD in humans [2, 4] but it induces only a first-degree heart block in Emd^{y/-} mice [106, 107]. Haploinsufficiency and point mutations in LMNA lead to EDMD in humans [5] and Lmna^{-/-} mice have severe abnormalities of both skeletal and cardiac muscles [101]. We show in the present invention that ERK is activated in hearts from Lmna^{-/-} mice compared to control mice. However, this appears to be a less pronounced activation than in hearts from Emd^{-/y} and Lmna^{H222P/H222P} mice [69, 89]. The cardiac phenotype in these three mouse models of EDMD is different. The Emd^{-/y} mice we analyzed have only minimal cardiac dysfunction characterized by first-degree heart block and vacuolization of cardiomyocytes and have normal life spans [106]. Lmna^{H222P/H222P} develop cardiac chamber dilation associated with decreased left ventricle fractional shortening starting at about 8 weeks of age and subsequently develop more severe conduction system abnormalities and dilated cardiomyopathy, dying at an average age of 36 weeks [14]. Lmna^{-/-} mice develop cardiac disease at 4 weeks of age with atrophic and degenerated myocytes and die at an average age of 8 weeks [101, 108]. We hypothesized a relationship between the degree of MAP kinase cascade activation and the severity of the heart disease [69]. Our present results suggest that this might not be the case. ERK activation in the heart is related to the development of cardiac dysfunction but other factors or signaling pathways could determine its progression or severity. This could explain why Emd^{-/y} mice have an apparently greater activation of ERK than Lmna^{-/-} mice. We have reported that other signaling cascades may be altered in hearts from Emd^{-/y} and male Lmna^{H222P/H222P} mice [69]. Among them are Wnt signaling pathway, I-κB/NF-κB cascade and Tgf-β receptor signaling pathway. These pathways may not be viewed as unique cascades, as crosstalks between Wnt, Tgf-β and MAP kinase pathways occur [109-112]. Hence, other signaling pathways could interact with ERK activation in the development of cardiac disease in X-linked and autosomal EDMD.

We detected ERK activation in hearts of Lmna^{-/-} mice at 5 weeks of age, which mice develop cardiomyopathy at 4 weeks of age [108]. A recent publication by Wolf et al. [113] described cardiac abnormalities in Lmna^{+/-} mice. These heterozygous null mice develop cardiac conduction defects at 10 weeks of age and dilated cardiomyopathy at approximately 50 weeks of age. The authors apparently did not observe an abnormal activation of MAP kinases and downstream targets in hearts from Lmna^{+/-} mice at 20 weeks [113]. They concluded that lamin haploinsufficiency does not cause activation of the ERK or JNK branches of the MAP kinase pathway. These results are not incompatible with those in our current or previous [69, 89] studies. Firstly, Lmna^{-/-} mice at 20 weeks do not have left ventricular dilation and do not develop it for another 30 weeks. It is therefore possible that ERK activation occurs sometime between 20 and 50 weeks of age prior to the onset of cardiomyopathy, as in Lmna H222P mice [89]. Secondly, Wolf et al. [113] showed an unchanged expression of ERK1/2 and JNK1 but did not clearly report data on levels of the phosphorylated (activated) forms of the proteins. It is therefore possible that their methods missed to detect activation of ERK and JNK. Finally, Wolf et al. [113] examined MAP kinase activities only in two wild type mice, giving their reported negative result low, if any, statistical power. Differences and similarities in MAP kinase activities in hearts from various mouse models of EDMD and cultured cells with similar genetic alterations remain to be further examined.

We have demonstrated that activation of MAP kinase pathway is related to abnormalities in nuclear envelope in transfected cultured cells expressing lamin A variants found in subjects with EDMD [89]. While most of the human LMNA mutations causing EDMD are missense, some patients carry nonsense mutations leading to haploinsufficiency [5]. Mutations in EMD on the other hand lead in most cases to a loss of emerin [2, 4]. Here we show an aberrant increase of ERK activation and downstream transcription factors in siRNA-treated HeLa and C2C12 cells with decreased emerin and A-type lamin expression. These results reproducibly show that altering the expression of A-type lamins and emerin activates MAP kinases [69. 89]. The MAP kinase cascade is a signal transduction pathway that transmits signals from extracellular stimuli such as growth factors and hormones [114] and from intracellular stimuli such as redox state [115]. In the cardiac cells, MAP kinases are stimulated by G-protein-coupled receptors (endothelin-1, α-adrenoreceptor agonists, angiotensin II), as well as mechanical stretch (structural stress and electrical pacing), H₂O₂ and osmotic shock [116]. Recent studies have identified a physical link between the nuclear envelope and the cytoplasm; the LILAC complex [117]. The LILAC complex provides a mechanical network from the cell surface to the nucleus. Reduced expression of A-type lamins and emerin could weaken the LILAC complex and make cells more susceptible to mechanical stress, in turn more readily leading to MAP kinase activation. However, the mechanisms that activate MAP kinases in cells with abnormalities in A-type lamins and emerin remain to be determined experimentally.

Our results have practical implications because small molecule drugs can be used to inhibit ERK [118]. Several ERK and JNK inhibitors, including PD98059 and SP600125 are commercially available, potent and selective inhibitors. PD98059 mediates its inhibitory properties by binding to MEK, therefore preventing phosphorylation of ERK. We show that PD98059 reduces ERK1/2 activity in HeLa and C2C12 cells with reduced A-type lamins and emerin. This opens the road for other similar studies on cultured cells committed to striated muscle lineages such as differentiated myotubes, mouse muscle satellite cells, mouse cardiac muscle cells and primary cardiomyocytes. Such studies could determine if activation of ERK due to reduced expression of A-type lamins and emerin is related to changes in the expression of downstream genes in muscle development or function in vitro. Furthermore, our results provide the basis for a “clinical trial” of an ERK inhibitor in a mouse model of cardiomyopathy.

### EXAMPLES

The following examples are meant to illustrate the methods and materials of the present invention and are not intended to limit the invention in any way.

### Example 1

### Activation of MAPK Pathways Links LMNA Mutations to Cardiomyopathy in Emery-Dreifuss Muscular Dystrophy

### Methods

### Mice

Lmna H222P knock-in mice were generated and genotyped as described (14). Hearts were isolated from male Lmna^{H222P/H222P}, Lmna^{H222P/+} and Lmna^{+/+} mice at 4, 7 or 10 weeks of age. For all immunoblotting and real-time PCR experiments, Lmna^{H222P/H222P} and Lmna^{H222P/+} mice were compared directly to Lmna^{+/+} littermates. For microarray analysis, mice were combined from 5 different litters of crosses between Lmna^{H222P/+} mice; control Lmna^{+/+} mice were included from each of the litters from which Lmna^{H222P/H222P} and Lmna^{H222P/+} were used.

### RNAi Isolation

Total RNA was extracted using the Rneasy isolation kit (Qiagen) according to the manufacturer's instructions. Adequacy and integrity of extracted RNA were determined by gel electrophoresis and concentrations measured by ultraviolet absorbance spectroscopy.

### Microarray Processing

We used Mouse Genome 430 2.0 GeneChip Arrays (Affymetrix), which contain 45,101 probes sets corresponding to known genes and expressed sequence tags. Complimentary DNA synthesis, cRNA synthesis and labeling were performed as described in the Affymetrix GeneChip Technical Manual. Hybridization, washing, staining and scanning of arrays were performed at the Gene Chip Core Facility of the Columbia University Genome Center.

### Microarray Data Analysis

Image files were obtained through Affymetrix GeneChip software and analyzed by robust multichip analysis using Affymetrix microarray “.cel” image file and GeneTraffic (Iobion Informatics) software. Robust multichip analysis is composed of three steps: background correction, quantile normalization and robust probe set summary. Genes were identified as differentially expressed if they met a false discovery rate threshold of 0.05 in a two-sample t-test (q-value) and showed at least a two-fold difference in expression independent of absolute signal intensity. We have made the gene expression data available in the National Center for Biotechnology Information's Gene Expression Omnibus (GEO, http://www.ncbi.nlm.nih.gov/geo/), accessible through GEO Series accession number GSE6397 and GSE6398.

### Analysis of Functional Groups of Genes

Gene expression changes related to functional groups were analyzed using the Class Score method in ermineJ to provide a statistical confidence to functional groupings (65). The algorithm takes as input the log-transformed t-test p-values of genes that are members of a single Gene Ontology class and estimates the probability that the set of q-values would occur by chance. Significant Gene Ontology terms were identified using a false discovery rate of 0.05. For automated functional annotation and classification of genes of interest based on GO terms we used the Database for Annotation, Visualization and Integrated Discovery (DAVID) (http://david.abcc.ncifcrf.gov/) (66).

### Real-Time PCR Analysis

We synthesized cDNA using Omniscript Reverse Transcriptase (Qiagen) on total cellular RNA. For each replicate in each experiment, RNA from tissue samples of different animals was used. Primers were designed correspond to mouse RNA sequences using Primer3 (http://frodo.wi.mit.edu/cgi-bin/primer3/primer3_www.cgi). The Real-time PCR reaction contained iQ SYBR green super mix (Bio-Rad), 200 nM of each primer and 0.2 μl of template in a 25-μl reaction volume. Amplification was carried out using the MyiQ Single-Color Real-Time PCR Detection System (Bio-Rad) with an initial denaturation at 95° C. for 2 min followed by 50 cycles at 95° C. for 30 s and 62° C. for 30 s. Relative levels of mRNA expression were calculated according to the ΔΔC_{T} method (67). Individual expression values were normalized by comparison with Gapdh mRNA.

Extraction of Proteins from Hearts and Immunoblotting

Hearts were excised from mice and snap-frozen in liquid nitrogen-cooled isopentane. To obtain protein extracts, both ventricles were homogenized in extraction buffer (25 mM Tris [pH 7.4], 150 mM NaCl, 5 mM EDTA, 10 mM sodium pyrophosphate, 1 mM Na₃VO₄, 1% SDS, 1 mM dithiothreitol) containing protease inhibitors (25 mg/ml aprotinin and 10 mg/ml leupeptin). Protein samples were subjected to SDS-PAGE, transferred to nitrocellulose membranes and blotted with primary Abs against elk-1 (Santa-Cruz), ERK1/2 (Santa-Cruz), pERK1/2 (Cell Signaling), JNK1 (Santa-Cruz), pJNK (Cell Signaling), bcl-2 (Santa-Cruz), pc-Jun (Santa-Cruz), β-MHC (Santa-Cruz), MLC-2 (Santa-Cruz), ANF (Santa-Cruz), β- actin (Santa-Cruz) and β-tubulin (Santa-Cruz). Secondary Abs were HRP—conjugated (Amersham). Recognized proteins were visualized by enhanced chemiluminescence (ECL-Amersham). Antibodies against β-tubulin and β-actin were used as internal controls to normalize the amounts of protein between immunoblots. Band densities were calculated using Scion Image software (Scion Corporation) and normalized to the appropriate total extract to control for protein loading. Data are reported as means±standard deviations and are compared with respective controls using a two-tailed t test.

### Immunohistochemistry

Immunofluorescence staining for pERK1/2 was performed on Frozen sections (8 μm) of transversal cardiac muscles by fixing them in 3.7% formaldehyde in PBS for 15 minutes, then blocked in 5% fetal goat serum in PBS/triton for 1 hour. Cells were incubated in blocking solution with anti-pERK1/2 monoclonal antibody (Cell Signaling) overnight at 4° C. followed by PBS washing and incubation with Texas red-conjugated goat anti-mouse IgG secondary antibody (Invitrogen) and counterstained with 0.1 μg/ml DAPI (Sigma-Aldrich). Intensity of pERK1/2 in cardiocytes was measured using Scion Image software (Scion Corporation). Data are reported as means±standard deviations and are compared with respective controls using a two-tailed t test.

### Primary Culture and Isolation of Ventricular Myocytes

Lmna^{-/+} and Lmna^{H222P/H222P} knock-in mice (10 weeks of age) were anesthetized with pentofurane. Ventricular cardiomyocytes were isolated as described in the Alliance for Cellular Signaling procedure protocol PP00000125 (http://www.signaling-gateway.org). For immunoblotting, cells were washed in ice cold PBS and lysed in extraction buffer. Lysates were centrifuged at 16,000×g and the supernatants collected Immunoblotting was performed as described above.

### Plasmid Construction

To generate constructs to express lamins in transfected cells, cDNAs encoding wild type lamin A and lamin A with H222P, N195K, R298C, R482W, N456I and T528K aa substitutions were cloned in pegfp-C1 plasmid (Clontech) between XhoI and BamHI restriction endonuclease sites.

### Cell Culture and Transfection to Examiner MAPK Activation and Localization

Cos-7 and C2C12 cells were maintained in Dulbecco's modified Eagle's medium supplemented with 10% fetal calf serum and 0.5% gentamycin at 37° C. in a humidified atmosphere of 95% air and 5% CO₂. Cells were transfected with plasmids encoding GFP-wild type lamin A and GFP-H222P lamin A using Lipofectamine 2000 according to the manufacturer's instructions (Invitrogen). Cells were analyzed 48 hours after transfection. Cells were either fixed for 10 min in methanol at −20° C. or lysed in extraction buffer for subsequent immunoblotting.

### Immunofluorescence Microscopy

For immunofluorescence staining, fixed cells were incubated with rabbit Abs that recognize pERK1/2 (Cell Signaling) or pJNK (Cell Signaling). Cells were then washed and incubated with Texas Red conjugated goat anti-rabbit secondary Abs (Molecular Probes). For immunohistochemistry, frozen sections (8 μm) of transversal cardiac muscles were fixed in 3.7% formaldehyde in PBS for 15 minutes and then blocked in 5% fetal goat serum in PBS/Triton X-100 for 1 hour. Abs used for immunohistochemistry were primary rabbit anti-pERK1/2 (Cell Signaling) and secondary Texas Red conjugated goat anti-rabbit (Molecular Probes). Sections were counterstained with 0.1 μg/ml DAPI (Sigma-Aldrich). Immunofluorescence microscopy was performed on a Microphot SA (Nikon) microscope attached to a Spot RT Slide camera (Diagnostic Instruments). Images were processed using Adobe Photoshop 6.0 (Adobe Systems). Fluorescence intensity in cardiocytes was measured using Scion Image software (Scion Corporation). Data are reported as means±standard deviations and are compared with respective controls using a two-tailed t test.

### Luciferase Reporter Gene Assays

Luciferase reporter assays for c-Jun and Elk-1 activation were carried out using Path Detect In Vivo Signal Transduction Pathway Trans-Reporting System (Stratagene). Cos-7 cells were plated in 12 well plates. The following day, cells were transfected with pegfp-N1 constructs encoding wild type and mutant lamin A proteins, pFA2-cJun or pFA2-Elk-1 (Stratagene) and pFR-Luc (Stratagene) using Lipofectamine 2000. To correct for transfection efficiency, a plasmid encoding β-gal was co-transfected. After 24 h, cells were trypsinized and protein lysates obtained and extracted according to the manufacturer's instructions (Promega). Luciferase activity was measured with a luminometer.

Gene Expression Profiling in Hearts from Mice with Lmna H222P Mutation

To identify abnormal expression of genes involved in development of cardiomyopathy caused by Lmna mutation, we carried out a genome-wide RNA expression analysis in hearts from Lmna^{H222P/+} and Lmna^{H222P/H222P} mice. A detailed description of these mice has been previously published (14). Male Lmna^{H222P/H222P} mice develop cardiac chamber dilation, decreased left ventricle fractional shortening and hypokinesis detectable by echocardiography at 8 weeks of age. At 12 weeks of age, conduction system abnormalities become pronounced, characterized primarily by an increased PR interval on electrocardiograms. Histological analysis shows left ventricular fibrosis and fiber degeneration by 16 weeks of age along with atrial dilation. Male mice die between 4 and 9 months of age. To focus on primary events and avoid interference caused by fibrotic cells and non-specific tissue damage in hearts from older Lmna^{H222P/H222P} mice, we analyzed samples from mice at 10 weeks of age. There are no histological detectable abnormalities in hearts of mice at this age (see below). We also used hearts from heterozygous Lmna^{H222P/+} mice, which do not develop signs of cardiomyopathy until 24 months of age (Bonne et al., unpublished observation) and have normal life spans.

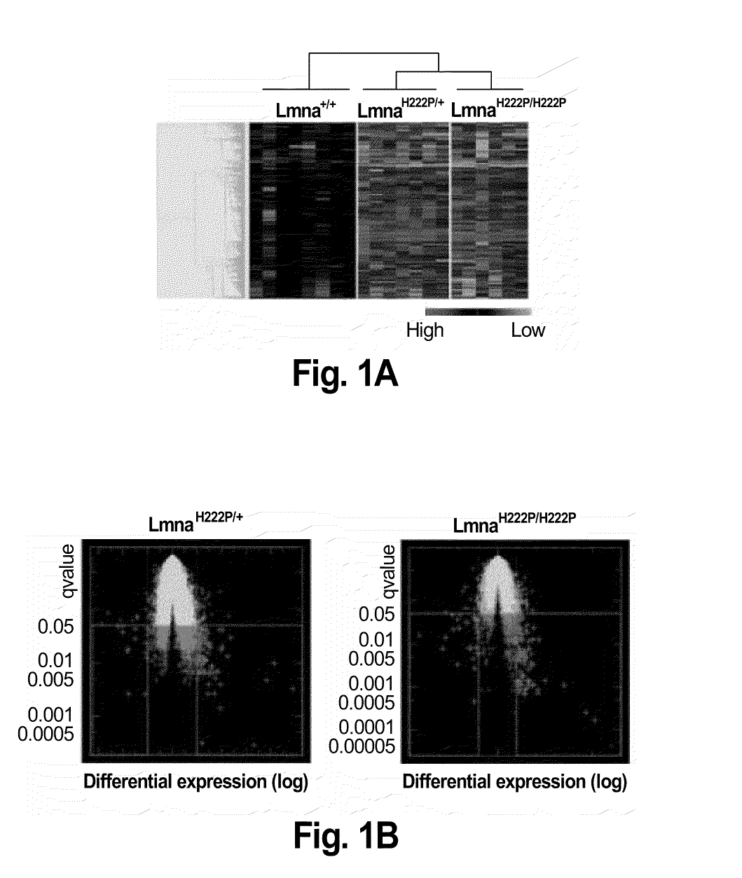

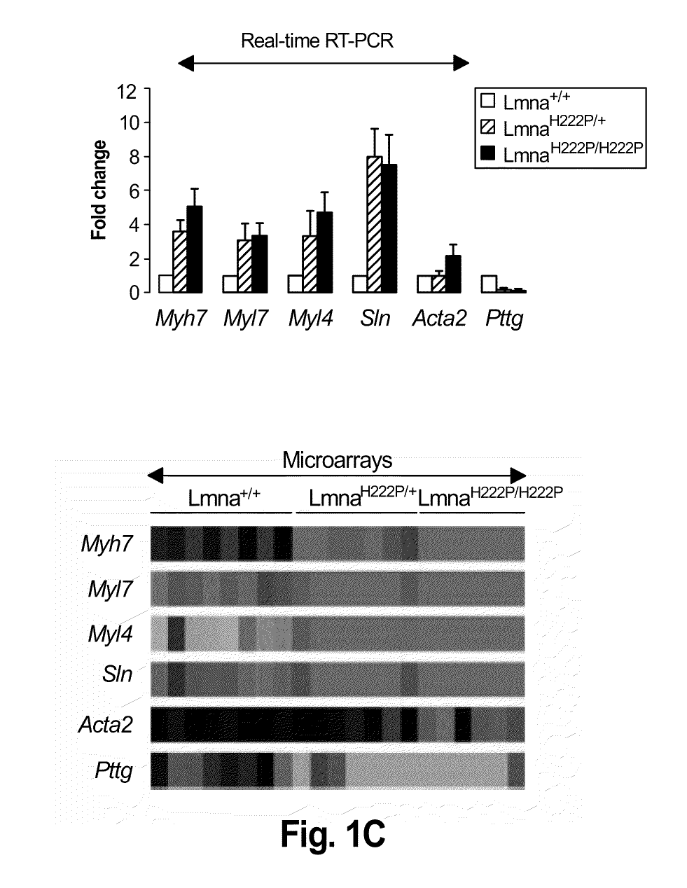

Hearts were isolated and transcription profiles determined using amplified RNA for microarray analyses. We used Affymetrix Mouse Genome 430 2.0 Arrays, which contain 45,101 probes sets for known and predicted genes. We examined similarities in transcription profiles between hearts from control Lmna^{+/+}, Lmna^{H222P/+} and Lmna^{H222P/H222P} mice by hierarchical cluster analysis. Using hearts from control mice (n=8) as a baseline, this analysis revealed a strong consistency between replicates and distinct patterns of gene expression (FIG. 1A). Compared to the mean value of expression in controls, hearts from Lmna^{H222P/H222P} (n=6) and Lmna^{H222P/+} (n=7) mice exhibited a large cluster of genes with increased expression and a small cluster with decreased expression.

We used a supervised learning method to distinguish probe sets representing genes with significant differences in expression between hearts from control and mutant mice. Probe sets were selected using sufficiently high absolute changes measured by q-values (q<0.05), which were determined using gene-wise t-tests. The analysis was tuned such that the false discovery rate among probe sets identified as significant was 5% and expression was more than 2-fold different than control. This analysis yielded 104 probe sets in hearts from Lmna^{H222P/+} mice and 114 in hearts from Lmna^{H222P/H222P} mice (FIG. 1B). The 104 probes sets identified in hearts from Lmna^{H222P/+} mice corresponded to 92 up-regulated genes, 69 known ones and 23 cDNAs with unknown functions (Table 1). The 12 down-regulated genes included 6 known ones and 6 uncharacterized cDNAs. The number of up-regulated genes corresponding to the probe sets identified in hearts from Lmna^{H222P/H222P} mice was 94, 73 known genes and 21 cDNAs of unknown function (Table 2). The number of down-regulated genes was 20, 8 known genes and 12 cDNAs with unknown functions. There were 57 similar probes sets between hearts from Lmna^{H222P/H222P} and Lmna^{H222P/+} mice (Table 3).

To validate expression of selected transcripts identified by microarray analysis, we performed real-time PCR using RNA extracted from mouse hearts (FIG. 1C). Genes encoding heavy and light chains of myosins (Myl7, Myl4, Myh7), actin-α2 (Acta2), sarcolipin (Sln) and pituitary tumor-transforming 1 (Pttg) were selected as representative. There was a correlation between real-time PCR results and altered expression detected by microarrays for these genes with greater than 2-fold differences in expression in hearts from both Lmna^{H222P/+} and Lmna^{H222P/H222P} mice (FIG. 1C).

Many genes, including some muscle-specific genes, with significantly altered expression compared to controls in hearts from Lmna^{H222P/+} and Lmna^{H222P/H222P} mice were identical (Table 3). Myl4, Myl7, Myh7 and Sln were up-regulated in hearts from both Lmna^{H222P/H222P} and Lmna^{H222P/+} mice. There was also increased expression of genes encoding LIM domain family members, including Pdlim3 and Fhl1. However, it appeared that increased expression of muscle-specific genes was greater in hearts of Lmna^{H222P/H222P} mice than from Lmna^{H222P/+} mice (Table 2 and Table 1, respectively). Statistically significant increases in RNA transcripts encoding atrial natriuretic factor and actin-α2 were observed only in hearts from Lmna^{H222P/H222P} mice (Table 2).

We used ermineJ software, which analyzes Gene Ontology terms applied to genes, to identify functional classes of genes differentially expressed in hearts from Lmna^{H222P/+} and Lmna^{H222P/H222P} mice compared to controls. Analysis using functional class scoring improves sensitivity by statistically evaluating genes in biologically meaningful groups. In hearts from Lmna^{H222P/H222P} mice, the highest scoring Gene Ontology classes were genes encoding proteins involved in inflammation and fibrosis (Table 4). However, these classes were not significantly altered in hearts from Lmna^{H222P/+} mice. Differential expression of genes encoding muscle components, including myosins and sarcomeric proteins, achieved statistical significance in hearts from Lmna^{H222P/H222P} and Lmna^{H222P/+} mice. Genes encoding various proteins involved in transcription and translation also demonstrated significant differences in expression, some only in hearts of Lmna^{H222P/H222P} and others in heterozygotes. Genes encoding proteins in the Wnt receptor signaling pathway, in heterotrimeric G-protein complexes, in the JNK cascade branch of the MAPK pathway, with protein phosphatase type 2A activities and with transmembrane receptor protein kinase activities demonstrated significantly altered expression in hearts from Lmna^{H222P/+} and Lmna^{H222P/H222P} mice (Table 4).

Analysis of Markers of Cardiomyopathy in Hearts from Mice with Lmna H222P Mutation

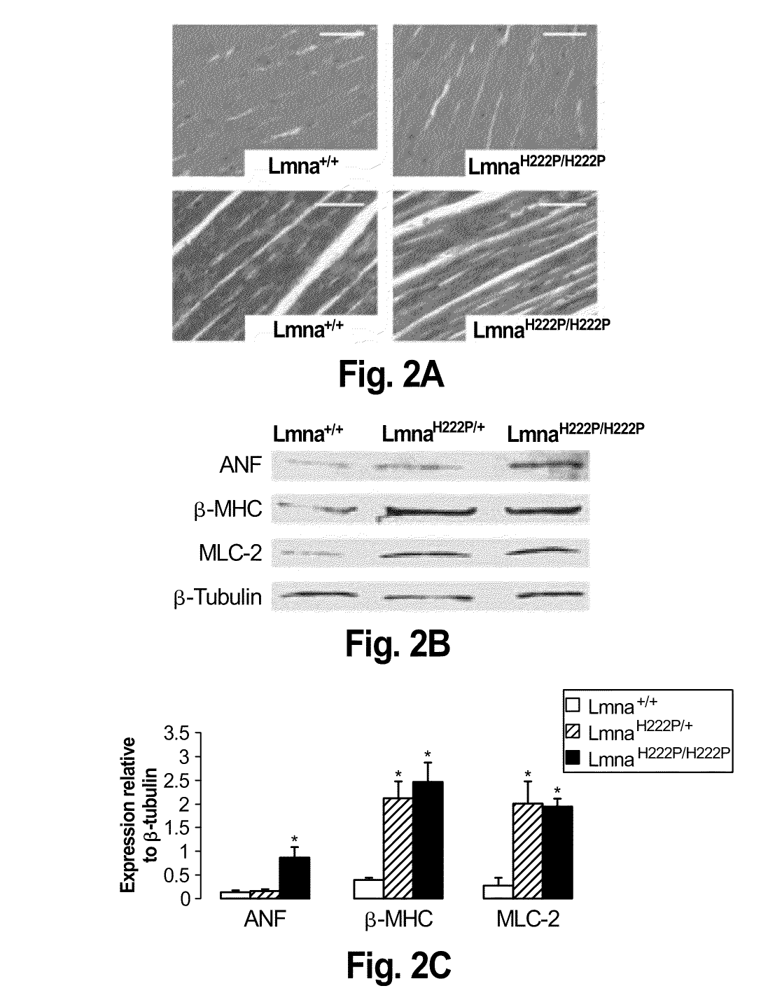

Several genes discovered to be differentially expressed in hearts of Lmna H222P knock-in mice using microarray analysis appeared to be involved in pathological changes of cardiomyopathy. Activation of genes encoding proteins involved in inflammation and fibrosis has been reported in previous studies of cardiomyopathies in humans and mice (15, 16) and agrees with pathological changes that develop in hearts of Lmna^{H222P/H222P} mice (14). However, histological examination did not reveal inflammation or fibrosis in hearts from Lmna^{H222P/H222P} mice at 10 weeks (FIG. 2A). This suggests that detection of “molecular signatures” using microarrays is more sensitive than conventional histology in detecting inflammation and fibrosis.

Deregulation of genes encoding muscle components and involved in “muscle organization” has been described in other studies of dilated cardiomyopathies (17, 18). We therefore used immunoblotting to analyze expression of β-myosin heavy chain (β-MHC) and myosin light chain 2 (MLC-2) polypeptides, as their genes showed significantly increased expression in hearts from mutant mice. Hearts from Lmna^{H222P/+} and Lmna^{H222P/H222P} mice respectively had 5.3-fold and 6.1-fold increases in β-MHC and 7.1-fold and 6.8-fold increases in MLC-2 expression compared to controls (FIG. 2B). We also measured expression of atrial natriuretic factor (ANF), which is up-regulated in heart failure, and its expression was increased in hearts from Lmna^{H222P/H222P} mice by approximately 6-fold compared to controls (FIG. 2B). However, the increase of ANF was not statistically significant in hearts from Lmna^{H222P/+} mice. This is consistent with results of microarray analysis and also cardiac chamber dilation in hearts of Lmna^{H222P/H222P} mice at 8 weeks of age (14).

### MAPK Pathways Involved in Development of Cardiomyopathy Are Activated in Hearts of Lmna H2222P Mice

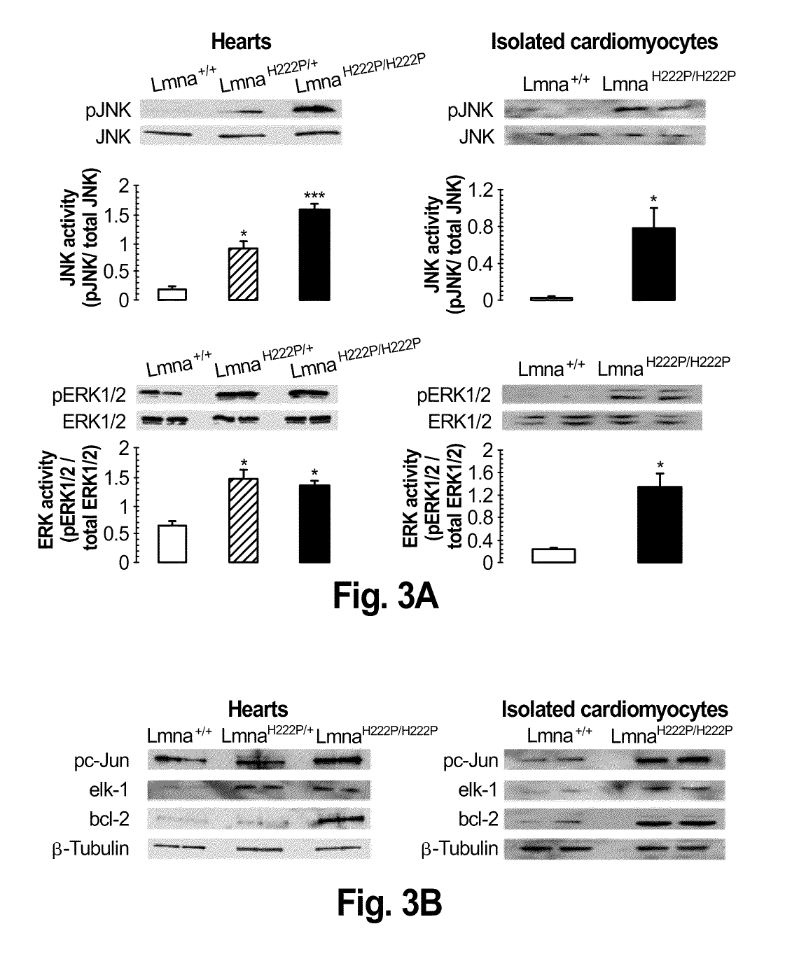

Our Functional Class Scoring analysis revealed significant differences in expression of genes encoding proteins in MAPK pathways in Lmna H222P mouse hearts (Table 4). Individual genes in MAPK pathways with significantly different expressions (q<0.05) in hearts from Lmna^{H222P+} and Lmna^{H222P/H222P} mice, as identified using DAVID (http://david.abcc.ncifcrf.gov/), are listed in Table 5 and Table 6. Because enhanced JNK cascade activity, a branch of MAPK pathways, has been shown to cause cardiomyopathy and conduction defects (19, 20), we focused our attention on the MAPK pathways. We first evaluated phosphorylation of two MAPKs, JNK and ERK1/2 (pJNK and pERK1/2, respectively), in hearts from Lmna^{+/+}, Lmna^{H222P/+} and Lmna^{H222P/H222P} mice. These kinases are activated by phosphorylation Immunoblotting with anti-pJNK Ab demonstrated 5-fold and 9-fold increases in pJNK in hearts from Lmna^{H222P/+} and Lmna^{H222P/H222P}, respectively (FIG. 3A). Phosphorylated ERK1/2 was also significantly increased in hearts from Lmna^{H222P/+} and Lmna^{H222P/H222P} mice (2.3-fold and 2.1-fold, respectively) (FIG. 3A). Infiltration of cells other than cardiomyocytes could be a variable accounting for detection of activated MAPKs in heart tissue. To remove the influence of such cells, we tested whether JNK and ERK1/2 kinases were activated in isolated ventricular cardiomyocytes from Lmna^{H222P/H222P}. Expressions of pERK1/2 and pJNK were increased 4-fold and 12-fold, respectively, in cardiomyocytes from Lmna^{H222P/H222P} mice compared to those from Lmna^{+/+} mice (FIG. 3A).

Phosphorylated JNK and pERK1/2 activate a series of downstream target genes, including those encoding bcl-2, elk-1 and c-Jun (21, 22, 23) Immunoblotting with Abs against bcl-2 and elk-1 demonstrated increased expression of these proteins in hearts from both Lmna^{H222P/H222P} and Lmna^{H222P/+} mice compared to Lmna^{+/+} mice (FIG. 3B). Pc-Jun was also increased in hearts from Lmna^{H222P/H222P} but not Lmna^{H222P/+} mice (FIG. 3B). Increases in elk-1, bcl-2 and pc-Jun were also detected in isolated ventricular cardiomyocytes from Lmna^{H222P/H222P} mice compared to controls (FIG. 3B). These data indicate aberrant activation of MAPK signaling in hearts from both Lmna^{H222P/H222P} and Lmna^{H222P/+} mice. However, the degree of enhanced signaling appeared to be greater in hearts from homozygous mutant mice.

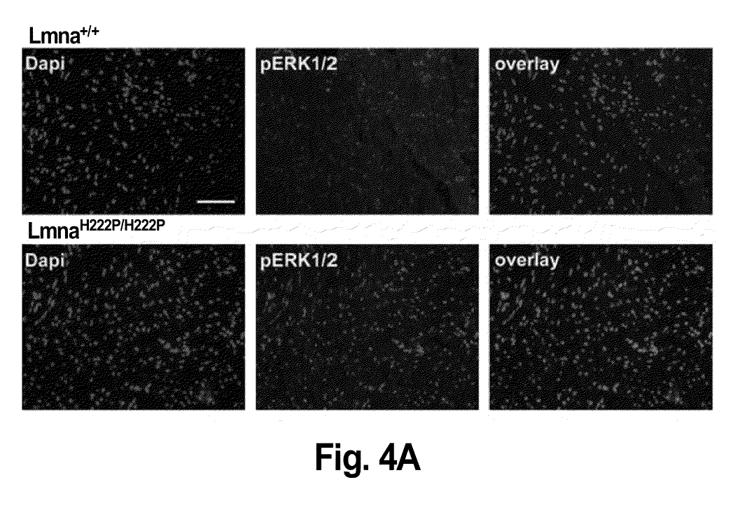

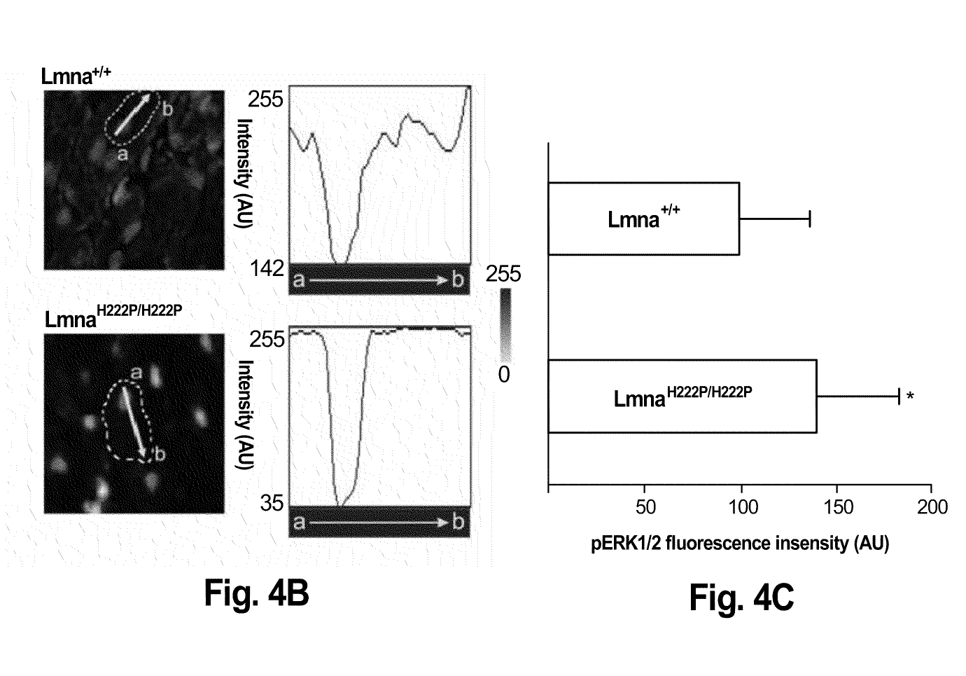

To analyze in vivo activation of MAPK, we used an Ab that recognized pERK1/2 in sections of heart tissue Immunofluorescence staining of heart sections from Lmna^{+/+} mice with these antibodies revealed a faint, rather diffuse pattern whereas fluorescence in hearts from Lmna^{H222P/H222P} mice was more intense and predominantly nuclear (FIG. 4A). Quantitative analysis of individual cardiomyocytes in the sections confirmed that anti-pERK Ab labeled both cytoplasm and nucleus in hearts from Lmna^{+/+} mice but essentially only the nucleus in hearts from Lmna^{H222P/H222P} mice (FIG. 4B). Fluorescence intensity of nuclear labeling was significantly higher in heart cells in Lmna^{H222P/H222P} mice compare to Lmna^{+/+} mice (FIG. 4C). These results demonstrate greater activation and nuclear translocation of a MAPK in hearts from Lmna^{H222P/H222P} mice compared to control.

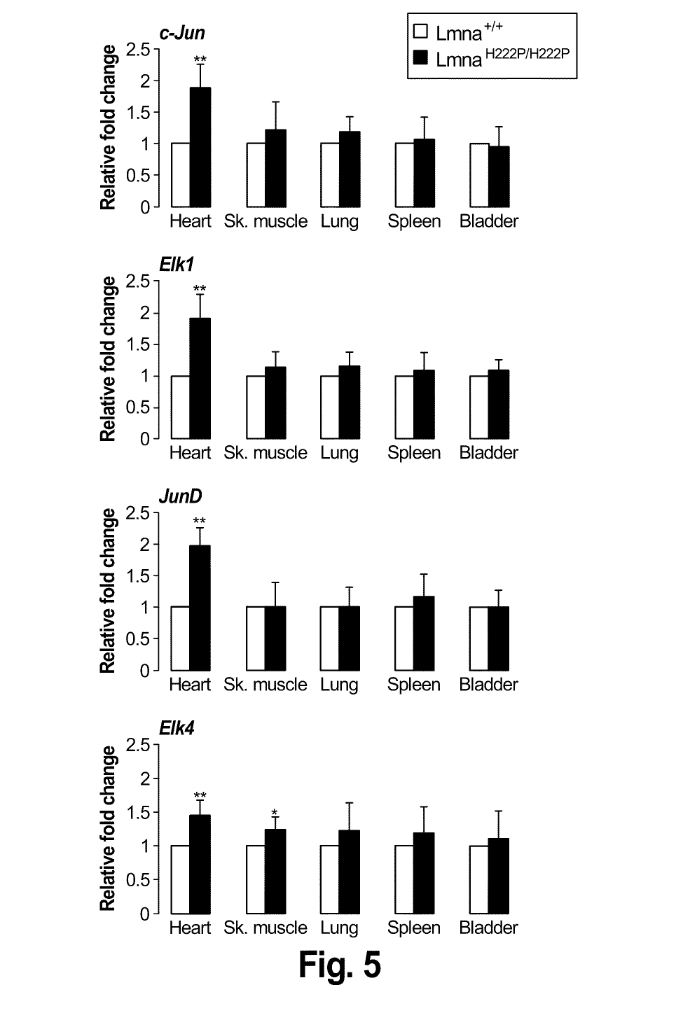

To determine if MAPK activation is observed only in heart, we measured expression of downstream target genes Elk-1, JunD, c-Jun and Elk-4 in different tissues. Real-time PCR showed significantly increased expression mostly in hearts from Lmna^{H222P/H222P} mice (FIG. 5). There was also increased expression of Elk-4 in skeletal muscle of Lmna^{H222P/H222P} mice.

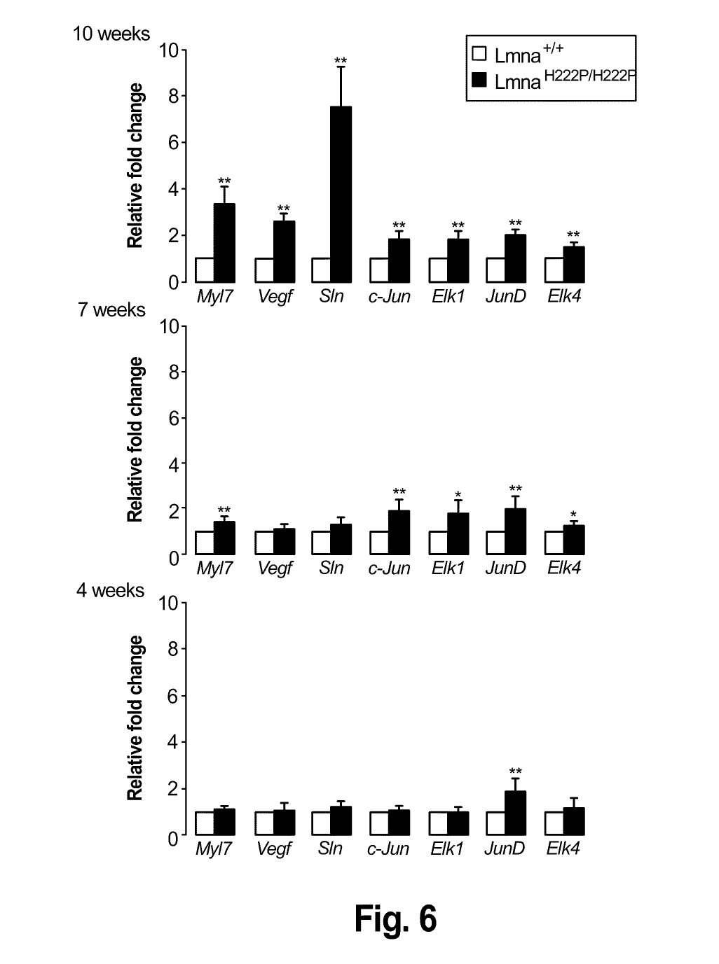

Our initial analyses used hearts from mice 10 weeks of age, when Lmna^{H222P/H222P} mice already have slight ventricular dilatation of the ventricles (14). Activation of fibrosis genes was also detected at this time. These alterations could affect cardiac cells and secondarily stimulate MAPK cascades. We therefore use real-time PCR to analyze expression of c-Jun, Elk-1, JunD and Elk-4 in hearts from Lmna^{+/+} and Lmna^{H222P/H222P} mice at 4, 7 and 10 weeks of age. In hearts from 10-week old Lmna^{H222P/H222P} mice, there was activation of Vegf, a marker of fibrosis, as well as Myl7 and Sln. Expression of c-Jun, Elk-1, JunD and Elk-4 was also significantly increased (FIG. 6). In 7-week old Lmna^{H222P/H222P} mice, there was increased cardiac expression of Myl7 but not Vegf or Sln and expression of c-Jun, Elk-1, JunD and Elk-4 was still increased (FIG. 6). At 4 weeks of age, there was only increased cardiac JunD expression in Lmna^{H222P/H222P} mice (FIG. 6). These results show that MAPK activation precedes increased expression of a fibrosis marker and two muscle-specific genes in hearts from Lmna^{H222P/H222P} mice.

A fetal-like gene expression program of genes encoding cytoskeletal proteins is characteristic of many types of cardiomyopathy (17, 18, 46-49) and is similarly initiated during cardiac remodeling due to mechanical strain, such in hypertensive cardiomyopathy (50, 51). Increases in ventricular expression of ANF have been documented in experimental models of heart failure and cardiomyopathy (29, 38, 52-55) as well as in human heart failure (56). Our analysis identified 104 and 114 genes that were differentially expressed in hearts from Lmna^{H222P/+} and Lmna^{H222P/H222P} mice, respectively. These included several in the cardiac fetal gene expression program, such as those encoding β-MHC (Myh7), MLC-2 (Myl4, Myl7) and ANF (Anf). However, levels in the changes in expression of these genes were different in hearts from Lmna^{H222P/+} and Lmna^{H222P/H222P} mice. For example, the log₂-fold changes in expression of Myh7 were 2.38 in hearts from Lmna^{H222P/+} mice and 3.49 in hearts from Lmna^{H222P/H222P}. Log₂-fold changes in expression of Myl4 were 3.84 in hearts from Lmna^{H222P/+} and 4.66 in hearts from Lmna^{H222P/H222P} mice. Anf was not differentially expressed in hearts from Lmna^{H222P/+} mice compared to wild type mice but was up-regulated significantly in hearts from Lmna^{H222P/H222P} mice. These different degrees of gene expression changes may reflect the early onset of heart failure in Lmna^{H222P/H222P} mice, which develop symptoms at approximately 2 months of age. In contrast, Lmna^{H222P/+} mice exhibit a decrease in left ventricular fractional shortening only at 2 years of age (Bonne et al., unpublished observation).

Expression of genes encoding skeletal muscle α-actin (57) and c-myc (58), which re-express during cardiac remodeling, was not increased in hearts from Lmna^{H222P/+} and Lmna^{H222P/H222P} ice. These genes are activated in an early-response against passive tension, for example in cardiac hypertrophy secondary to pressure overload. Lack of activation of these genes is consistent with dilated cardiomyopathy without cellular hypertrophy and disarray in hearts from Lmna^{H222P/H222P} mice (14). The observed up-regulation of genes encoding extracellular matrix-proteins, such as those encoding collagen Iα2 (Colla2), decorin (Dcn) and matrix metalloproteinase 14 (Mmp14), may underlie the development of fibrosis in hearts from Lmna^{H222P/H222P} mice.

Expression of Lamin A with the H222P aa Substitution Activates JNK and ERK and Alters Subcellular Localization

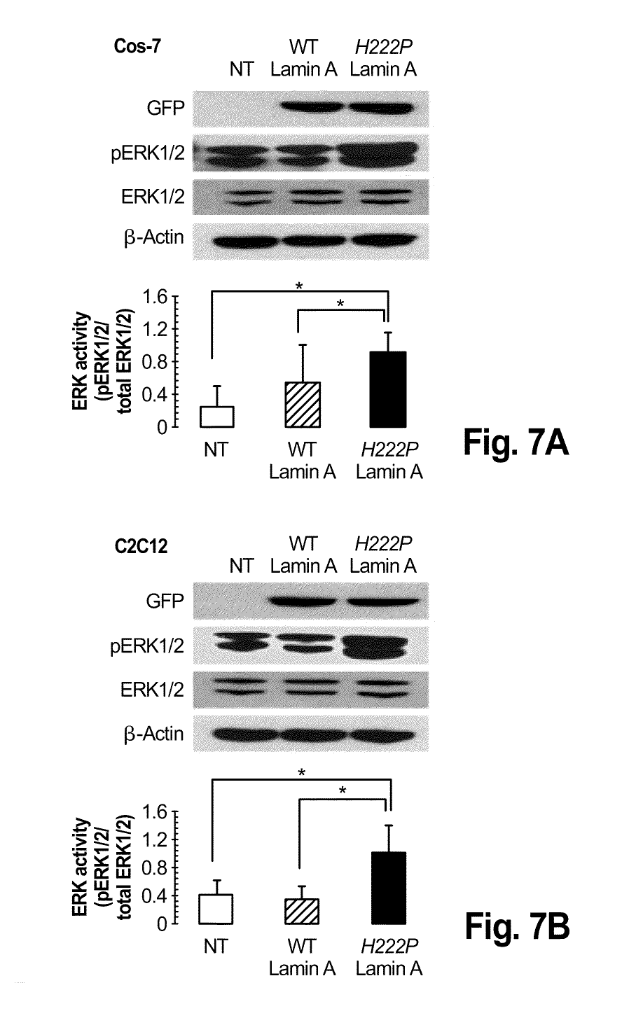

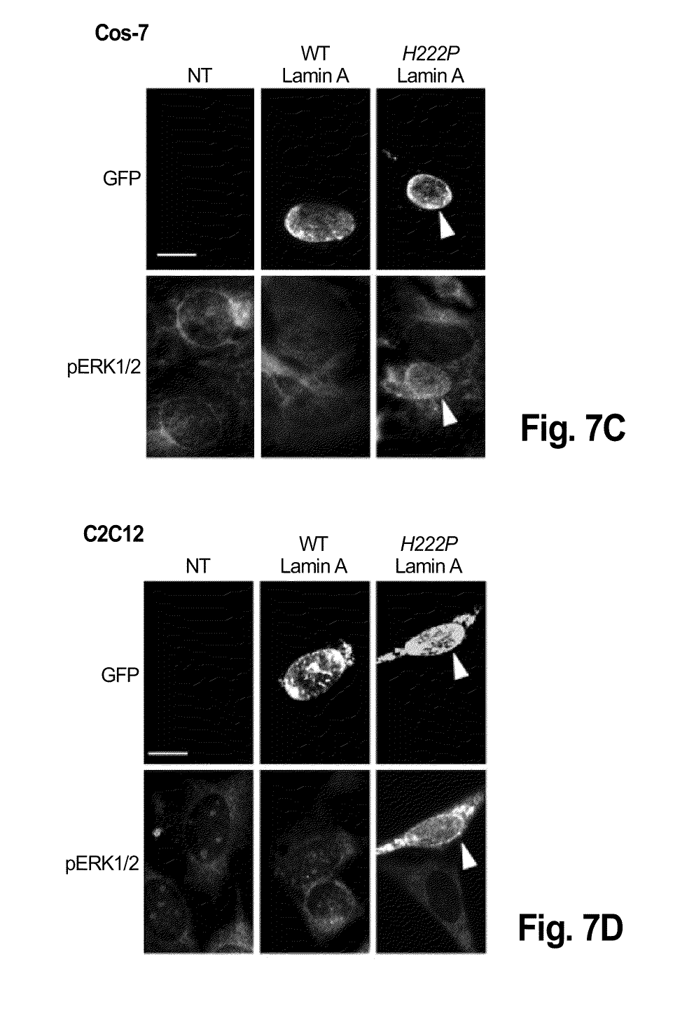

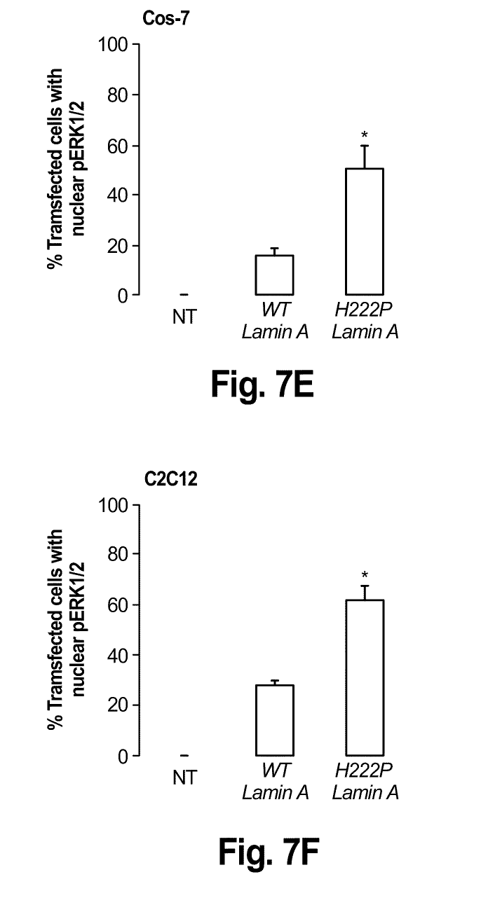

To determine if expression of lamin A with the H222P aa substitution is responsible for activation of MAPK signaling, we measured pERK1/2 and pJNK in transiently transfected Cos-7 and C2C12 cells expressing GFP fusions of wild type and H222P lamin A Immunoblotting with Abs against total ERK1/2 and pERK1/2 demonstrated that expression of H222P lamin A increased the amount of phosphorylated protein (FIG. 7A and FIG. 7B). The increase was significant compared to non-transfected cells and cells expressing the GFP fusion of wild type lamin A.

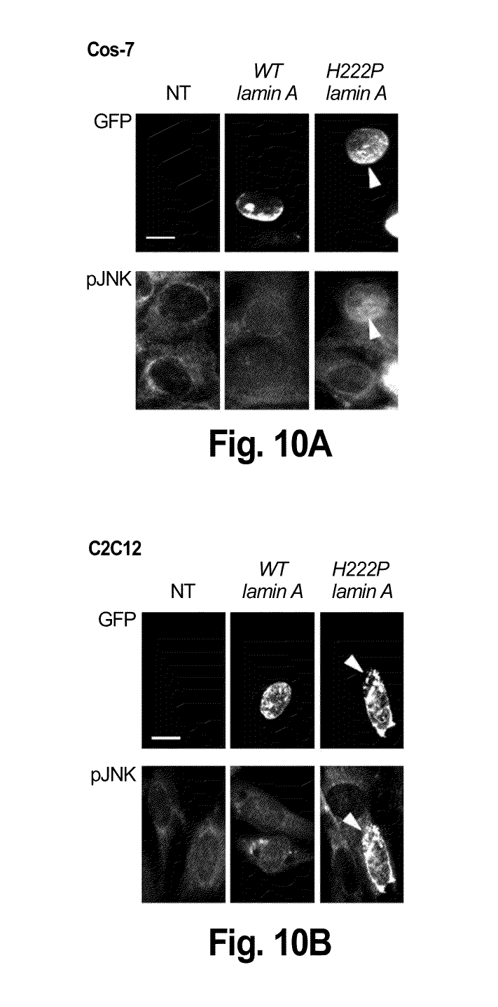

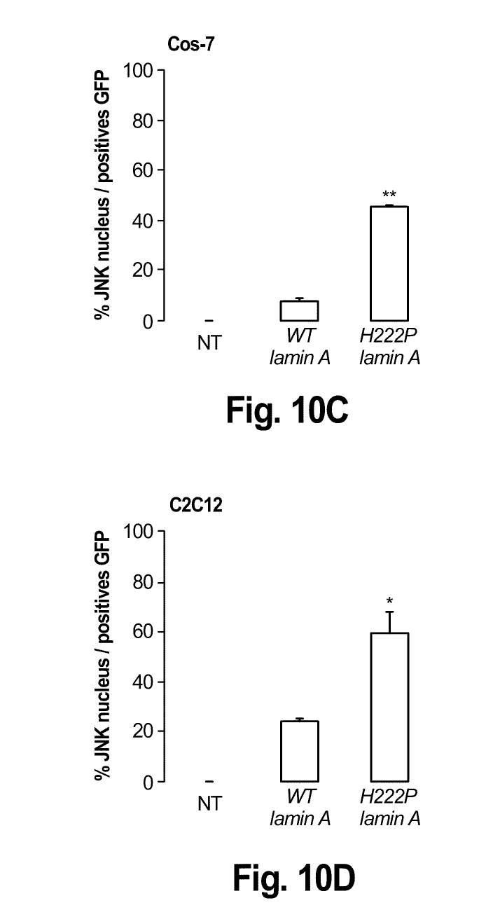

Translocation of pERK1/2 and pJNK from cytoplasm to nucleus is required for activation of downstream targets (24, 25). In non-transfected Cos-7 and C2C12 cells, pERK1/2 was mainly distributed in the cytoplasm (FIG. 7C and FIG. 7D). When transfected with a plasmid expressing the GFP fusion of wild type lamin A, pERK1/2 was also mainly distributed in cytoplasm (FIGS. 7C and 7D). In contrast, expression of a GFP fusion of H222P lamin A induced translocation of pERK1/2 into the nucleus (FIG. 7C and FIG. 7D). Approximately 80% of Cos-7 and C2C12 cells expressing H222P lamin A showed a nuclear localization of pERK1/2. Nuclear localization of pERK1/2 was observed in only 15% of Cos-7 cells and 30% of C2C12 cells expressing wild type lamin A and was not observed in untransfected cells (FIG. 7E and FIG. 7F). Similar results were obtained for pJNK (FIG. 10). Hence, expression of H222P lamin A stimulates phosphorylation and nuclear translocation of JNK and ERK1/2. While MAPKs were activated in C2C12 myoblasts transfected with H222P Lamin A, activated MAPK was not detected in skeletal muscle from Lmna^{H222P/H222P} mice. However, myoblasts are only a small component of heterogeneous skeletal muscle sections. Furthermore, in humans with EDMD as well as Lmna^{H222P/H222P} mice, skeletal muscle is variably affected.

Expression of Lamin A with aa Substitutions Identified in EDMD Associated with Cardiomyopathy Activates JNK and ERK

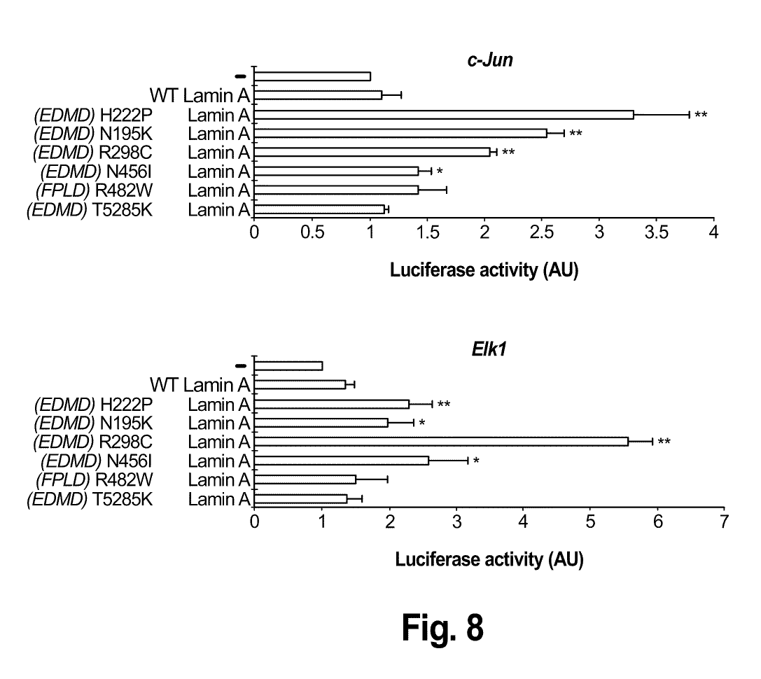

To further evaluate the effects of lamin A mutants on activation of MAPK pathways, we examined expression of c-Jun and Elk-1 reporter genes. We transiently transfected Cos-7 cells with plasmids encoding GFP fusions of H222P lamin A as well as wild type lamin A and 5 other lamin A mutants. Cells were simultaneously transfected with plasmids encoding a reporter system to detect c-Jun or Elk-1 promoter activities. Expression of H222P lamin A and lamin A with N195K, R298C, and N456I aa substitutions found in EDMD significantly increased activity of the c-Jun and Elk-1 promoters (FIG. 8). Overexpression of wild type lamin A and lamin A with a R482W mutation found in subjects with Dunnigan-type partial lipodystrophy did not significantly increase their activity. However, expression of one lamin A with an aa substitution that causes EDMD (T528K) did not significantly increase c-Jun and Elk-1 promoters activities in this assay. A possible explanation of this observation may be that a GFP-fusion of this mutant folds abnormally or is not as stable as the others when overexpressed in transfected cells. These results show that expression of A-type lamins with aa substitutions encoded by LMNA mutations causing cardiomyopathy leads to stimulation of downstream target genes in MAPK cascades in cultured cells.

TABLE 1
Genes with altered expression as defined by q < 0.05 and >1 log₂-fold
change in hearts from Lmna^{H222P/+} mice.

| Probe set name | Gene symbol | Gene name | Fold | q-value |
|---|---|---|---|---|
| 1449071_at | Myl7 | myosin, light polypeptide 7, regulatory | 4.91 | 0.003262303 |
| 1420884_at | Sln | sarcolipin | 4.14 | 0.009783776 |
| 1422580_at | Myl4 | myosin, light polypeptide 4, alkali; atrial, | 3.84 | 0.004778844 |
| | | embryonic | | |
| 1425521_at | Paip1 | polyadenylate binding protein-interacting | 3.32 | 0.000279415 |
| | | protein 1 | | |
| 1448553_at | Myh7 | myosin, heavy polypeptide 7, cardiac | 2.38 | 0.029672983 |
| | | muscle, beta | | |
| 1449824_at | Prg4 | proteoglycan 4 | 2.25 | 0.01330861 |
| 1441679_at | Cacna1c | calcium channel, voltage-dependent, L | 2.04 | 0.041791634 |
| | | type, alpha 1C subunit | | |
| 1449434_at | Car3 | carbonic anhydrase 3 | 2.03 | 0.019735505 |
| 1419100_at | Serpina3n | serine (or cysteine) proteinase inhibitor, | 1.92 | 0.041583562 |
| | | clade A, member 3N | | |
| 1426260_a_at | Ugt1a6 | UDP glycosyltransferase 1 family, | 1.86 | 0.006793397 |
| | | polypeptide A6 | | |
| 1424749_at | Wdfy1 | WD repeat and FYVE domain containing 1 | 1.79 | 0.006916706 |
| 1449178_at | Pdlim3 | PDZ and LIM domain 3 | 1.70 | 0.002607879 |
| 1448595_a_at | Rex3 | reduced expression 3 | 1.65 | 0.006428347 |
| 1428484_at | Osbpl3 | oxysterol binding protein-like 3 | 1.61 | 0.008837797 |
| 1453232_at | Calr3 | calreticulin 3 | 1.58 | 0.007966835 |
| 1424454_at | A930025J12RIK | RIKEN cDNA A930025J12 gene | 1.55 | 0.019735505 |
| 1453145_at | 4933439C20RIK | RIKEN cDNA 4933439C20 gene | 1.53 | 0.006361691 |
| 1417462_at | Cap1 | CAP, adenylate cyclase-associated protein | 1.50 | 0.020072033 |
| | | 1 (yeast) | | |
| 1435176_a_at | Idb2 | inhibitor of DNA binding 2 | 1.49 | 0.006407563 |
| 1430519_a_at | Cnot7 | CCR4-NOT transcription complex, | 1.47 | 0.0039589 |
| | | subunit 7 | | |
| 1433184_at | 6720477C19RIK | RIKEN cDNA 6720477C19 gene | 1.45 | 0.035675657 |
| 1454959_s_at | Gnai1 | guanine nucleotide binding protein, alpha | 1.45 | 0.015512236 |
| | | inhibiting 1 | | |
| 1423915_at | 4832415H08RIK | RIKEN cDNA 4832415H08 gene | 1.45 | 0.002607879 |
| 1417867_at | I | adipsin | 1.45 | 0.023285335 |
| 1423954_at | C3 | complement component 3 | 1.39 | 0.024131569 |
| 1449461_at | Rbp7 | retinol binding protein 7, cellular | 1.39 | 0.042288878 |
| 1455136_at | Atp1a2 | ATPase, Na+/K+ transporting, alpha 2 | 1.38 | 0.006428347 |
| | | polypeptide | | |
| 1452417_x_at | AV057155 | AV057155 Mus musculus pancreas | 1.35 | 0.040758652 |
| | | C57BL/6J adult Mus musculus | | |
| 1443799_at | AV348753 | AV348753 RIKEN full-length enriched, | 1.35 | 0.029103142 |
| | | adult male olfactory | | |
| 1421551_s_at | Ifi202b | interferon activated gene 202B | 1.34 | 0.011539146 |
| 1449514_at | Gprk5 | G protein-coupled receptor kinase 5 | 1.32 | 0.023285335 |
| 1419527_at | Comp | cartilage oligomeric matrix protein | 1.31 | 0.032455373 |
| 1432205_a_at | C130038G02RIK | RIKEN cDNA C130038G02 gene | 1.30 | 0.00705383 |
| 1422651_at | Acdc | adipocyte, C1Q and collagen domain | 1.27 | 0.028936401 |
| | | containing | | |
| 1447640_s_at | Pbx3 | pre B-cell leukemia transcription factor 3 | 1.27 | 0.027186297 |
| 1427183_at | Efemp1 | epidermal growth factor-containing | 1.26 | 0.012371525 |
| 1448823_at | Cxcl12 | fibulin-like extracellular matrix protein 1 | 1.25 | 0.006196772 |
| | | chemokine (C—X—C motif) ligand 12 | | |
| 1421855_at | Fgl2 | fibrinogen-like protein 2 | 1.24 | 0.012386508 |
| 1420731_a_at | Csrp2 | cysteine and glycine-rich protein 2 | 1.24 | 0.011179832 |
| 1454966_at | AK031326 | unknown | 1.23 | 0.042632665 |
| 1427038_at | BC049766 | unknown | 1.22 | 0.007548482 |
| 1437123_at | Mmrn2 | multimerin 2 | 1.21 | 0.000313227 |
| 1418674_at | Osmr | oncostatin M receptor | 1.20 | 0.026645199 |
| 1415994_at | Cyp2e1 | cytochrome P450, family 2, subfamily e, | 1.19 | 0.044394162 |
| | | polypeptide 1 | | |
| 1428343_at | C730034d20rik | RIKEN cDNA C730034D20 gene | 1.19 | 0.018171429 |
| 1420930_s_at | Catnal1 | catenin alpha-like 1 | 1.19 | 0.03182764 |
| 1455812_x_at | Slitl2 | Slit-like 2 (Drosophila) | 1.18 | 0.023909901 |
| 1421163_a_at | Nfia | nuclear factor I/A | 1.18 | 0.034980094 |
| 1423854_a_at | BC008101 | unknown | 1.17 | 0.009795877 |
| 1427660_x_at | D6MIT97 | DNA segment, Chr 6, Massachusetts | 1.17 | 0.038499517 |
| | | Institute of Technology 97 | | |
| 1424383_at | BC003277 | cDNA sequence BC003277 | 1.16 | 0.009154495 |
| 1420952_at | Son | Son cell proliferation protein | 1.16 | 0.046071596 |
| 1417126_a_at | 3110001N18RIK | RIKEN cDNA 3110001N18 gene | 1.15 | 0.009282894 |
| 1440335_at | AV020525 | AV020525 Mus musculus 18-day embryo | 1.13 | 0.046559367 |
| | | C57BL/6J Mus musculus | | |
| 1416666_at | Serpine2 | serine (or cysteine) proteinase inhibitor, | 1.13 | 0.013133867 |
| | | Glade E, member 2 | | |
| 1451447_at | C330016O16RIK | RIKEN cDNA 0330016O16 gene | 1.13 | 0.012386508 |
| 1426208_x_at | Plagl1 | pleiomorphic adenoma gene-like 1 | 1.12 | 0.022034481 |
| 1448669_at | Dkk3 | dickkopf homolog 3 (Xenopus laevis) | 1.12 | 0.032901376 |
| 1429197_s_at | BC038651 | unknown | 1.12 | 0.006916706 |
| 1436672_at | BB766329 | BB766329 RIKEN full-length enriched, | 1.12 | 0.017740892 |
| | | B16 F10Y cells Mus | | |
| 1448734_at | Cp | ceruloplasmin | 1.11 | 0.021961097 |
| 1418021_at | Slp | sex-limited protein | 1.11 | 0.034532714 |
| 1434975_x_at | 9030221M09RIK | RIKEN cDNA 9030221M09 gene | 1.11 | 0.019735505 |
| 1426851_a_at | Nov | nephroblastoma overexpressed gene | 1.11 | 0.033160989 |
| 1450876_at | Cfh | complement component factor h | 1.10 | 0.035303157 |
| 1449556_at | H2-T23 | histocompatibility 2, T region locus 23 | 1.10 | 0.027018405 |
| 1422631_at | Ahr | aryl-hydrocarbon receptor | 1.09 | 0.006196772 |
| 1433647_s_at | Rhobtb3 | Rho-related BTB domain containing 3 | 1.09 | 0.017793681 |
| 1433525_at | Ednra | endothelin receptor type A | 1.09 | 0.02333671 |
| 1419155_a_at | Sox4 | SRY-box containing gene 4 | 1.09 | 0.027469092 |
| 1419130_at | Deadc1 | deaminase domain containing 1 | 1.09 | 0.006602357 |
| 1453435_a_at | Fmo2 | flavin containing monooxygenase 2 | 1.09 | 0.020289251 |
| 1417065_at | Egr1 | early growth response 1 | 1.08 | 0.021448558 |
| 1449106_at | Gpx3 | glutathione peroxidase 3 | 1.08 | 0.030677188 |
| 1448162_at | Vcam1 | vascular cell adhesion molecule 1 | 1.08 | 0.026413922 |
| 1422715_s_at | Acp1 | acid phosphatase 1, soluble | 1.07 | 0.023907748 |
| 1423753_at | Bambi | BMP and activin membrane-bound | 1.06 | 0.011539146 |
| | | inhibitor, homolog (Xenopus laevis) | | |
| 1430637_at | 2210016H18RIK | RIKEN cDNA 2210016H18 gene | 1.06 | 0.047172391 |
| 1434990_at | Ak122434 | unknown | 1.06 | 0.006916706 |
| 1451240_a_at | Glo1 | glyoxalase 1 | 1.05 | 0.042857264 |
| 1421955_a_at | Nedd4 | neural precursor cell expressed, | 1.05 | 0.035645582 |
| | | developmentally down-regulated gene 4 | | |
| 1436431_at | 1700025G04RIK | RIKEN cDNA 1700025G04 gene | 1.05 | 0.011155847 |
| 1418536_at | H2-Q7 | histocompatibility 2, Q region locus 7 | 1.04 | 0.033908679 |
| 1451285_at | Fus | fusion, derived from t(12;16) malignant | 1.04 | 0.022761224 |
| | | liposarcoma (human) | | |
| 1435943_at | Dpep1 | dipeptidase 1 (renal) | 1.03 | 0.010803727 |
| 1448705_at | Zfp297 | zinc finger protein 297 | 1.03 | 0.014276303 |
| 1438631_x_at | BC017545 | unknown | 1.03 | 0.021575905 |
| 1456226_x_at | Ddr1 | discoidin domain receptor family, | 1.03 | 0.021976404 |
| | | member 1 | | |
| 1417872_at | Fhl1 | four and a half LIM domains 1 | 1.03 | 0.009154583 |
| 1455940_x_at | Wdr6 | WD repeat domain 6 | 1.03 | 0.01925172 |
| 1434328_at | Loc380747 | similar to 60S ribosomal protein L15 | 1.02 | 0.02321354 |
| 1419103_a_at | Abhd6 | abhydrolase domain containing 6 | 1.02 | 0.00503353 |
| 1438754_at | Av372127 | AV372127 RIKEN full-length enriched, | −1.02 | 0.043783361 |
| | | adult male colon Mus | | |
| 1447802_x_at | AV099323 | expressed sequence AV099323 | −1.03 | 0.012371525 |
| 1452590_a_at | BC032982 | unknown | −1.08 | 0.015451729 |
| 1434008_at | Loc384934 | similar to sodium channel beta 4 subunit | −1.08 | 0.023285335 |
| 1451675_a_at | Alas2 | aminolevulinic acid synthase 2, erythroid | −1.10 | 0.002607879 |
| 1452318_a_at | M12573 | unknown | −1.51 | 0.043962944 |
| 1426607_at | 3110070M22RIK | RIKEN cDNA 3110070M22 gene | −1.51 | 0.028191889 |
| 1418480_at | Cxcl7 | chemokine (C—X—C motif) ligand 7 | −1.78 | 0.002607879 |
| 1437721_at | BB543398 | BB543398 RIKEN full-length enriched, 0 | −1.80 | 0.018132165 |
| | | day neonate eyeball | | |
| 1422919_at | Hrasls | HRAS-like suppressor | −2.02 | 0.037734414 |
| 1438390_s_at | Pttg1 | pituitary tumor-transforming 1 | −2.05 | 0.049414848 |
| 1428347_at | Cyfip2 | cytoplasmic FMR1 interacting protein 2 | −2.22 | 0.04909376 |

TABLE 2
Genes with altered expression as defined by q < 0.05 and >1 log₂-fold change in
hearts from Lmna^{H222P/H222P} mice.

| Probe set name | Gene symbol | Gene name | Fold | q-value |
|---|---|---|---|---|
| 1449071_at | Myl7 | myosin, light polypeptide 7, regulatory | 6.02 | 4.16256E−05 |
| 1420884_at | Sln | sarcolipin | 5.13 | 0.000169726 |
| 1422580_at | Myl4 | myosin, light polypeptide 4, alkali; | 4.66 | 0.000256675 |
| | | atrial, embryonic | | |
| 1448553_at | Myh7 | myosin, heavy polypeptide 7, cardiac | 3.49 | 0.001102336 |
| | | muscle, beta | | |
| 1453898_at | AK009352 | unknown | 2.50 | 0.001402809 |
| 1449434_at | Car3 | carbonic anhydrase 3 | 2.17 | 0.012439184 |
| 1457666_s_at | Ifi202b | interferon activated gene 202B | 2.11 | 0.000846853 |
| 1448595_a_at | Rex3 | reduced expression 3 | 2.10 | 0.000715362 |
| 1425521_at | Paip1 | polyadenylate binding protein- | 1.89 | 0.023572154 |
| | | interacting protein 1 | | |
| 1449824_at | Prg4 | proteoglycan 4 | 1.85 | 0.003308574 |
| 1456062_at | Anf | atrial natriuretic factor | 1.84 | 0.001119551 |
| 1418701_at | Arvcf | armadillo repeat gene deleted in velo- | 1.83 | 0.012252465 |
| | | cardio-facial syndrome | | |
| 1419100_at | Serpina3n | serine (or cysteine) proteinase inhibitor, | 1.77 | 0.005743576 |
| | | clade A, member 3N | | |
| 1454959_s_at | Gnai1 | guanine nucleotide binding protein, | 1.74 | 0.003649336 |
| | | alpha inhibiting 1 | | |
| 1429196_at | BC038651 | unknown | 1.73 | 0.003065936 |
| 1437358_at | Wdfy1 | WD repeat and FYVE domain | 1.65 | 0.001337287 |
| | | containing 1 | | |
| 1419155_a_at | Sox4 | SRY-box containing gene 4 | 1.63 | 0.001102336 |
| 1448669_at | Dkk3 | dickkopf homolog 3 (Xenopus laevis) | 1.57 | 0.00096279 |
| 1455136_at | Atp1a2 | ATPase, Na+/K+ transporting, alpha 2 | 1.57 | 0.001402809 |
| | | polypeptide | | |
| 1450857_a_at | Col1a2 | procollagen, type I, alpha 2 | 1.55 | 0.026789241 |
| 1428484_at | Osbpl3 | oxysterol binding protein-like 3 | 1.48 | 0.020741743 |
| 1425394_at | BC023105 | cDNA sequence BC023105 | 1.45 | 0.006458272 |
| 1430519_a_at | Cnot7 | CCR4-NOT transcription complex, | 1.45 | 0.004940192 |
| | | subunit 7 | | |
| 1420731_a_at | Csrp2 | cysteine and glycine-rich protein 2 | 1.44 | 0.000911703 |
| 1432205_a_at | C130038G02RIK | RIKEN cDNA C130038G02 gene | 1.42 | 0.000671074 |
| 1424383_at | BC003277 | cDNA sequence BC003277 | 1.41 | 0.000846853 |
| 1448823_at | Cxcl12 | chemokine (C—X—C motif) ligand 12 | 1.39 | 0.000715362 |
| 1435290_x_at | H2-Aa | histocompatibility 2, class II antigen A, | 1.39 | 0.003976722 |
| | | alpha | | |
| 1449178_at | Pdlim3 | PDZ and LIM domain 3 | 1.37 | 0.005362642 |
| 1429060_at | D830013H23RIK | RIKEN cDNA D830013H23 gene | 1.37 | 0.004876867 |
| 1421855_at | Fgl2 | fibrinogen-like protein 2 | 1.33 | 0.005743576 |
| 1425425_a_at | Wif1 | Wnt inhibitory factor 1 | 1.33 | 0.040656125 |
| 1428343_at | C730034D20RIK | RIKEN cDNA C730034D20 gene | 1.32 | 0.001873588 |
| 1437401_at | Igf1 | insulin-like growth factor 1 | 1.32 | 0.001699794 |
| 1435176_a_at | Idb2 | inhibitor of DNA binding 2 | 1.30 | 0.001735733 |
| 1417065_at | Egr1 | early growth response 1 | 1.29 | 0.012103309 |
| 1419527_at | Comp | cartilage oligomeric matrix protein | 1.28 | 0.015363433 |
| 1416666_at | Serpine2 | serine (or cysteine) proteinase inhibitor, | 1.28 | 0.016566752 |
| | | clade E, member 2 | | |
| 1426208_x_at | Plagl1 | pleiomorphic adenoma gene-like 1 | 1.28 | 0.013612184 |
| 1449106_at | Gpx3 | glutathione peroxidase 3 | 1.27 | 0.001946526 |
| 1449368_at | Dcn | decorin | 1.25 | 0.012103309 |
| 1418174_at | Dbp | D site albumin promoter binding protein | 1.25 | 0.005860191 |
| 1423753_at | Bambi | BMP and activin membrane-bound | 1.23 | 0.001119551 |
| | | inhibitor, homolog (Xenopus laevis) | | |
| 1425519_a_at | Ii | Ia-associated invariant chain | 1.22 | 0.005689275 |
| 1453145_at | 4933439C20RIK | RIKEN cDNA 4933439C20 gene | 1.22 | 0.020741743 |
| 1423854_a_at | BC008101 | unknown | 1.21 | 0.004003416 |
| 1427038_at | BC049766 | unknown | 1.21 | 0.024041634 |
| 1448162_at | Vcam1 | vascular cell adhesion molecule 1 | 1.19 | 0.017776185 |
| 1439766_x_at | Vegfc | vascular endothelial growth factor C | 1.19 | 0.000846853 |
| 1451447_at | C330016O16RIK | RIKEN cDNA C330016O16 gene | 1.18 | 0.017776185 |
| 1419130_at | Deadc1 | deaminase domain containing 1 | 1.18 | 0.013925793 |
| 1437224_at | Rtn4 | reticulon 4 | 1.18 | 0.006437433 |
| 1420952_at | Son | Son cell proliferation protein | 1.17 | 0.002274827 |
| 1416454_s_at | Acta2 | actin, alpha 2, smooth muscle, aorta | 1.17 | 0.001385314 |
| 1437056_x_at | 1810049K24RIK | RIKEN cDNA 1810049K24 gene | 1.17 | 0.041776172 |
| 1448416_at | Mglap | matrix gamma-carboxyglutamate (gla) | 1.16 | 0.004940192 |
| | | protein | | |
| 1448383_at | Mmp14 | matrix metalloproteinase 14 | 1.16 | 0.045912269 |
| | | (membrane-inserted) | | |
| 1447640_s_at | Pbx3 | pre B-cell leukemia transcription factor 3 | 1.15 | 0.035771251 |
| 1415859_at | 3230401O13RIK | RIKEN cDNA 3230401O13 gene | 1.15 | 0.023410072 |
| 1418532_at | Fzd2 | frizzled homolog 2 (Drosophila) | 1.15 | 0.007687062 |
| 1460049_s_at | 1500015O10R1K | RIKEN cDNA 1500015O10 gene | 1.15 | 0.045369376 |
| 1449461_at | Rbp7 | retinol binding protein 7, cellular | 1.12 | 0.022192646 |
| 1451567_a_at | Ifi203 | interferon activated gene 203 | 1.11 | 0.017776185 |
| 1422607_at | Etv1 | ets variant gene 1 | 1.11 | 0.004940192 |
| 1424186_at | 2610001E17RIK | RIKEN cDNA 2610001E17 gene | 1.11 | 0.049669589 |
| 1426851_a_at | Nov | nephroblastoma overexpressed gene | 1.11 | 0.023572154 |
| 1417462_at | Cap1 | CAP, adenylate cyclase-associated | 1.10 | 0.033983868 |
| | | protein 1 (yeast) | | |
| 1426510_at | C330023F11RIK | RIKEN cDNA C330023F11 gene | 1.10 | 0.036018704 |
| 1419042_at | AW111922 | expressed sequence AW111922 | 1.10 | 0.000756273 |
| 1437123_at | Mmrn2 | multimerin 2 | 1.10 | 0.000871383 |
| 1455346_at | Masp1 | mannan-binding lectin serine protease 1 | 1.10 | 0.007687062 |
| 1448288_at | E030026I10RIK | RIKEN cDNA E030026I10 gene | 1.09 | 0.004940192 |
| 1422631_at | Ahr | aryl-hydrocarbon receptor | 1.09 | 0.018968038 |
| 1433924_at | Peg3 | paternally expressed 3 | 1.08 | 0.035287641 |
| 1421917_at | Pdgfra | platelet derived growth factor receptor, | 1.08 | 0.012633389 |
| | | alpha polypeptide | | |
| 1422476_at | Ifi30 | interferon gamma inducible protein 30 | 1.08 | 0.022192646 |
| 1450648_s_at | H2-Ab1 | histocompatibility 2, class II antigen A, | 1.08 | 0.001119551 |
| | | beta 1 | | |
| 1441137_at | AK015956 | unknown | 1.08 | 0.035122609 |
| 1449514_at | Gprk5 | G protein-coupled receptor kinase 5 | 1.05 | 0.049669589 |
| 1417872_at | Fhl1 | four and a half LIM domains 1 | 1.05 | 0.001337287 |
| 1417025_at | H2-Eb1 | histocompatibility 2, class II antigen E | 1.05 | 0.004603993 |
| | | beta | | |
| 1443621_at | BG092359 | mac09f12.x1 Soares mouse 3NbMS | 1.04 | 0.001224434 |
| | | Mus musculus cDNA clone | | |
| 1426083_a_at | Btg1 | B-cell translocation gene 1, anti- | 1.04 | 0.016940974 |
| | | proliferative | | |
| 1449556_at | H2-T23 | histocompatibility 2, T region locus 23 | 1.03 | 0.02194224 |
| 1454696_at | BC003294 | unknown | 1.02 | 0.023314255 |
| 1456226_x_at | Ddr1 | discoidin domain receptor family, | 1.02 | 0.024041634 |
| | | member 1 | | |
| 1454764_s_at | Slc38a1 | solute carrier family 38, member 1 | 1.02 | 0.04708546 |
| 1418929_at | Esrrbl1 | estrogen-related receptor beta like 1 | 1.02 | 0.011056089 |
| 1455393_at | Cp | ceruloplasmin | 1.02 | 0.019001003 |
| 1448754_at | Rbp1 | retinol binding protein 1, cellular | 1.02 | 0.041776172 |
| 1439381_x_at | Mrvldc1 | MARVEL (membrane-associating) | 1.01 | 0.020452219 |
| | | domain containing 1 | | |
| 1447903_x_at | Ap1s2 | adaptor-related protein complex 1, | 1.01 | 0.031364482 |
| | | sigma 2 subunit | | |
| 1434141_at | Gucy1a3 | guanylate cyclase 1, soluble, alpha 3 | 1.01 | 0.046340908 |
| 1417253_at | Frg1 | FSHD region gene 1 | 1.01 | 0.013925793 |
| 1424505_at | 0610042C05RIK | RIKEN cDNA 0610042C05 gene | −1.02 | 0.018968038 |
| 1434008_at | Loc384934 | similar to sodium channel beta 4 subunit | −1.07 | 0.017776185 |
| 1456397_at | BB210819 | BB210819 RIKEN full-length enriched, | −1.08 | 0.013489859 |
| | | 0 day neonate thymus | | |
| 1434893_at | AI845177 | UI-M-BG0-aht-a-04-0-UI.s1 | −1.09 | 0.023572154 |
| | | NIH_BMAP_MSC Mus musculus | | |
| | | cDNA | | |
| 1451371_at | 1110025G12RIK | RIKEN cDNA 1110025G12 gene | −1.10 | 0.027434576 |
| 1452590_a_at | BC032982 | unknown | −1.11 | 0.005944427 |
| 1421278_s_at | Spna1 | spectrin alpha 1 | −1.12 | 0.033541643 |
| 1455208_at | Pex19 | peroxisome biogenesis factor 19 | −1.14 | 0.008107383 |
| 1452388_at | BC054782 | unknown | −1.15 | 0.035755727 |
| 1424077_at | 2610020H15RIK | RIKEN cDNA 2610020H15 gene | −1.23 | 0.002678732 |
| 1417680_at | Kcna5 | potassium voltage-gated channel, | −1.24 | 0.040656125 |
| | | shaker-related subfamily, member 5 | | |
| 1438511_a_at | 1190002H23RIK | RIKEN cDNA 1190002H23 gene | −1.25 | 0.000463385 |
| 1447802_x_at | AV099323 | expressed sequence AV099323 | −1.31 | 0.004421495 |
| 1457282_x_at | Tubgcp5 | tubulin, gamma complex associated | −1.45 | 0.007899515 |
| | | protein 5 | | |
| 1427126_at | M12573 | unknown | −1.91 | 0.046946097 |
| 1428991_at | Hrasls | HRAS-like suppressor | −2.07 | 0.003308574 |
| 1437721_at | BB543398 | BB543398 RIKEN full-length enriched, | −2.08 | 0.036438465 |
| | | 0 day neonate eyeball | | |
| 1424105_a_at | Pttg1 | pituitary tumor-transforming 1 | −2.69 | 0.013852722 |
| 1428347_at | Cyfip2 | cytoplasmic FMR1 interacting protein 2 | −2.84 | 0.007687062 |
| 1432198_at | 6330414G02R1K | RIKEN cDNA 6330414G02 gene | −4.12 | 0.015578517 |

TABLE 3
Genes commonly affected as defined by q < 0.05 and >1 log₂-fold change
in hearts from Lmna^{H222P/H222P} and Lmna^{H222P/+} mice.

| Probe set | Gene | | Lmna^{H222P/H222P} | | Lmna^{H222P/+} | |
|---|---|---|---|---|---|---|
| name | symbol | Gene name | Fold | q-value | Fold | q-value |
| 1449071_at | Myl7 | myosin, light polypeptide 7, | 6.02 | 4.16256E−05 | 4.91 | 0.003262303 |
| | | regulatory | | | | |
| 1420884_at | Sln | sarcolipin | 5.13 | 0.000169726 | 4.14 | 0.009783776 |
| 1422580_at | Myl4 | myosin, light polypeptide 4, alkali; | 4.66 | 0.000256675 | 3.84 | 0.004778844 |
| | | atrial, embryonic | | | | |
| 1448553_at | Myh7 | myosin, heavy polypeptide 7, | 3.49 | 0.001102336 | 2.38 | 0.029672983 |
| | | cardiac muscle, beta | | | | |
| 1449434_at | Car3 | carbonic anhydrase 3 | 2.17 | 0.012439184 | 2.03 | 0.019735505 |
| 1457666_s_at | Ifi202b | interferon activated gene 202B | 2.11 | 0.000846853 | 1.34 | 0.011539146 |
| 1448595_a_at | Rex3 | reduced expression 3 | 2.10 | 0.000715362 | 1.65 | 0.006428347 |
| 1425521_at | Paip1 | polyadenylate binding protein- | 1.89 | 0.023572154 | 3.32 | 0.000279415 |
| | | interacting protein 1 | | | | |
| 1449824_at | Prg4 | proteoglycan 4 | 1.85 | 0.003308574 | 2.25 | 0.01330861 |
| 1454959_s_at | Gnai1 | guanine nucleotide binding protein, | 1.74 | 0.003649336 | 1.45 | 0.015512236 |
| | | alpha inhibiting 1 | | | | |
| 1429196_at | BC038651 | unknown | 1.73 | 0.003065936 | 1.12 | 0.006916706 |
| 1437358_at | Wdfy1 | WD repeat and FYVE domain | 1.65 | 0.001337287 | 1.79 | 0.006916706 |
| | | containing 1 | | | | |
| 1419155_a_at | Sox4 | SRY-box containing gene 4 | 1.63 | 0.001102336 | 1.09 | 0.027469092 |
| 1448669_at | Dkk3 | dickkopf homolog 3 (Xenopus laevis) | 1.57 | 0.00096279 | 1.12 | 0.032901376 |
| 1455136_at | Atp1a2 | ATPase, Na+/K+ transporting, alpha | 1.57 | 0.001402809 | 1.38 | 0.006428347 |
| | | 2 polypeptide | | | | |
| 1428484_at | Osbpl3 | oxysterol binding protein-like 3 | 1.48 | 0.020741743 | 1.61 | 0.008837797 |
| 1430519_a_at | Cnot7 | CCR4-NOT transcription complex, | 1.45 | 0.004940192 | 1.47 | 0.0039589 |
| | | subunit 7 | | | | |
| 1420731_a_at | Csrp2 | cysteine and glycine-rich protein 2 | 1.44 | 0.000911703 | 1.24 | 0.011179832 |
| 1432205_a_at | C130038G02RIK | RIKEN cDNA C130038G02 gene | 1.42 | 0.000671074 | 1.30 | 0.00705383 |
| 1424383_at | BC003277 | cDNA sequence BC003277 | 1.41 | 0.000846853 | 1.16 | 0.009154495 |
| 1448823_at | Cxcl12 | chemokine (C-X-C motif) ligand 12 | 1.39 | 0.000715362 | 1.25 | 0.006196772 |
| 1449178_at | Pdlim3 | PDZ and LIM domain 3 | 1.37 | 0.005362642 | 1.70 | 0.002607879 |
| 1421855_at | Fgl2 | fibrinogen-like protein 2 | 1.33 | 0.005743576 | 1.24 | 0.012386508 |
| 1428343_at | C730034D20RIK | RIKEN cDNA C730034D20 gene | 1.32 | 0.001873588 | 1.19 | 0.018171429 |
| 1435176_a_at | Idb2 | inhibitor of DNA binding 2 | 1.30 | 0.001735733 | 1.49 | 0.006407563 |
| 1417065_at | Egr1 | early growth response 1 | 1.29 | 0.012103309 | 1.08 | 0.021448558 |
| 1419527_at | Comp | cartilage oligomeric matrix protein | 1.28 | 0.015363433 | 1.31 | 0.032455373 |
| 1416666_at | Serpine2 | serine (or cysteine) proteinase | 1.28 | 0.016566752 | 1.13 | 0.013133867 |
| | | inhibitor, clade E, member 2 | | | | |
| 1426208_x_at | Plagl1 | pleiomorphic adenoma gene-like 1 | 1.28 | 0.013612184 | 1.12 | 0.022034481 |
| 1449106_at | Gpx3 | glutathione peroxidase 3 | 1.27 | 0.001946526 | 1.08 | 0.030677188 |
| 1423753_at | Bambi | BMP and activin membrane-bound | 1.23 | 0.001119551 | 1.06 | 0.011539146 |
| | | inhibitor, homolog (Xenopus laevis) | | | | |
| 1453145_at | 4933439C20RIK | RIKEN cDNA 4933439C20 gene | 1.22 | 0.020741743 | 1.53 | 0.006361691 |
| 1423854_a_at | BC008101 | unknown | 1.21 | 0.004003416 | 1.17 | 0.009795877 |
| 1427038_at | BC049766 | unknown | 1.21 | 0.024041634 | 1.22 | 0.007548482 |
| 1448162_at | Vcam1 | vascular cell adhesion molecule 1 | 1.19 | 0.017776185 | 1.08 | 0.026413922 |
| 1451447_at | C330016O16RIK | RIKEN cDNA C330016O16 gene | 1.18 | 0.017776185 | 1.13 | 0.012386508 |
| 1419130_at | Deadc1 | deaminase domain containing 1 | 1.18 | 0.013925793 | 1.09 | 0.006602357 |
| 1420952_at | Son | Son cell proliferation protein | 1.17 | 0.002274827 | 1.16 | 0.046071596 |
| 1447640_s_at | Pbx3 | pre B-cell leukemia transcription | 1.15 | 0.035771251 | 1.27 | 0.027186297 |
| | | factor 3 | | | | |
| 1449461_at | Rbp7 | retinol binding protein 7, cellular | 1.12 | 0.022192646 | 1.39 | 0.042288878 |
| 1426851_a_at | Nov | nephroblastoma overexpressed gene | 1.11 | 0.023572154 | 1.11 | 0.033160989 |
| 1417462_at | Cap1 | CAP, adenylate cyclase-associated | 1.10 | 0.033983868 | 1.50 | 0.020072033 |
| | | protein 1 (yeast) | | | | |
| 1437123_at | Mmrn2 | multimerin 2 | 1.10 | 0.000871383 | 1.21 | 0.000313227 |
| 1422631_at | Ahr | aryl-hydrocarbon receptor | 1.09 | 0.018968038 | 1.09 | 0.006196772 |
| 1449514_at | Gprk5 | G protein-coupled receptor kinase 5 | 1.05 | 0.049669589 | 1.32 | 0.023285335 |
| 1417872_at | Fhl1 | four and a half LIM domains 1 | 1.05 | 0.001337287 | 1.03 | 0.009154583 |
| 1449556_at | H2-T23 | histocompatibility 2, T region locus 23 | 1.03 | 0.02194224 | 1.10 | 0.027018405 |
| 1456226_x_at | Ddr1 | discoidin domain receptor family, | 1.02 | 0.024041634 | 1.03 | 0.021976404 |
| | | member 1 | | | | |
| 1455393_at | Cp | ceruloplasmin | 1.02 | 0.019001003 | 1.11 | 0.021961097 |
| 1434008_at | Loc384934 | similar to sodium channel beta 4 | −1.07 | 0.017776185 | −1.08 | 0.023285335 |
| | | subunit | | | | |
| 1452590_a_at | BC032982 | unknown | −1.11 | 0.005944427 | −1.08 | 0.015451729 |
| 1447802_x_at | AV099323 | expressed sequence AV099323 | −1.31 | 0.004421495 | −1.03 | 0.012371525 |
| 1427126_at | M12573 | unknown | −1.91 | 0.046946097 | −1.51 | 0.043962944 |
| 1428991_at | Hrasls | HRAS-like suppressor | −2.07 | 0.003308574 | −2.02 | 0.037734414 |
| 1437721_at | BB543398 | BB543398 RIKEN full-length | −2.08 | 0.036438465 | −1.80 | 0.018132165 |
| | | enriched, 0 day neonate eyeball | | | | |
| 1424105_a_at | Pttg1 | pituitary tumor-transforming 1 | −2.69 | 0.013852722 | −2.05 | 0.049414848 |
| 1428347_at | Cyfip2 | cytoplasmic FMR1 interacting | −2.84 | 0.007687062 | −2.22 | 0.04909376 |
| | | protein 2 | | | | |

TABLE 4
Top scoring gene ontology (GO) terms listed with corresponding q-value and GO
identification numbers in hearts from Lmna^{H222P/H222P} and Lmna^{H222P/+} mice.

| | | q-value | |
|---|---|---|---|
| GO term | GO id | H222P/H222P | H222P/+ |
| Inflammation | | | |
| MHC class II receptor activity | GO: 0045012 | 0.00000015 | |
| antigen processing | GO: 0030333 | 0.00000022 | |
| antigen presentation | GO: 0019882 | 0.00000036 | |
| MHC class I receptor activity | GO: 0030106 | 0.00343037 | |
| complement activation | GO: 0006956 | 0.02981152 | |
| Fibrosis | | | |
| vascular endothelial growth factor receptor | GO: 0005021 | 0.00661132 | |
| activity | | | |
| Muscle Components | | | |
| contractile fiber | GO: 0043292 | 0.00005914 | 0.01837139 |
| sarcomere | GO: 0030017 | 0.00022889 | 0.03241135 |
| muscle myosin | GO: 0005859 | 0.00038115 | 0.04948603 |
| structural constituent of muscle | GO: 0008307 | 0.0005612 | 0.0474557 |
| Transcription/Translation | | | |
| poly(A) binding | GO: 0008143 | 0.0054132 | |
| specific RNA polymerase II transcription factor | GO: 0003704 | 0.0145961 | 0.01878892 |
| activity | | | |
| single-stranded DNA binding | GO: 0003697 | 0.01538049 | 0.03929412 |
| eukaryotic 43S preinitiation complex | GO: 0016282 | 0.02104322 | 0.04732741 |
| heterogeneous nuclear ribonucleoprotein | GO: 0030530 | 0.02280669 | |
| complex | | | |
| ATP-dependent RNA helicase activity | GO: 0004004 | 0.02928037 | 0.00315888 |
| transcriptional repressor complex | GO: 0017053 | 0.02961308 | 0.01050794 |
| tRNA ligase activity | GO: 0004812 | 0.03062475 | |
| double-stranded RNA binding | GO: 0003725 | 0.04470709 | 0.0136727 |
| regulation of translational initiation | GO: 0006446 | 0.04794275 | 0.01326877 |
| Signaling Pathways | | | |
| insulin-like growth factor binding | GO: 0005520 | 0.00044041 | |
| Ras protein signal transduction | GO: 0007265 | 0.00208363 | |
| Wnt receptor signaling pathway | GO: 0016055 | 0.00237991 | 0.0185489 |
| heterotrimeric G-protein complex | GO: 0005834 | 0.00414498 | 0.00283341 |
| JNK cascade | GO: 0007254 | 0.00526864 | 0.02433549 |
| MAP kinase activity | GO: 0004707 | 0.01475557 | |
| nuclear translocation of MAPK | GO: 0000189 | 0.02418454 | |
| protein phosphatase type 2A activity | GO: 0000158 | 0.03400446 | 0.02621094 |
| transmembrane receptor protein kinase activity | GO: 0019199 | 0.03446463 | 0.02434485 |

TABLE 5
Genes from MAPK pathways affected as defined by q < 0.05
in hearts from Lmna^{H222P/H222P} mice.

| Gene symbol | Gene name | q-value |
|---|---|---|
| Tgfb2 | transforming growth factor, beta 2 | 2.86E−07 |
| Fgf9 | fibroblast growth factor 9 | 5.67E−06 |
| Mapk8 | mitogen activated protein kinase 8 | 1.49E−05 |
| Evi1 | ecotropic viral integration site 1 | 3.05E−05 |
| Pdgfra | platelet derived growth factor receptor, alpha polypeptide | 4.59E−05 |
| Ddit3 | DNA-damage inducible transcript 3 | 4.63E−05 |
| Pdgfa | platelet derived growth factor, alpha | 1.61E−04 |
| Ikbkg | inhibitor of kappaB kinase gamma | 5.91E−04 |
| Rap1b | RAS related protein 1b | 6.48E−04 |
| Tgfbr2 | transforming growth factor, beta receptor II | 6.56E−04 |
| Rasa1 | RAS p21 protein activator 1 | 7.32E−04 |
| Map3k7 | mitogen activated protein kinase kinase kinase 7 | 8.20E−04 |
| Fgf12 | fibroblast growth factor 12 | 8.94E−04 |
| Flnb | filamin, beta | 0.001165294 |
| Tgfbr1 | transforming growth factor, beta receptor I | 0.001166535 |
| Rasa2 | RAS p21 protein activator 2 | 0.001215545 |
| Ppp3ca | protein phosphatase 3, catalytic subunit, alpha isoform | 0.001246963 |
| Stk4 | serine/threonine kinase 4 | 0.001354133 |
| Tgfb3 | transforming growth factor, beta 3 | 0.002364518 |
| Il1b | interleukin 1 beta | 0.002765324 |
| Stmn1 | stathmin 1 | 0.002968037 |
| Dusp9 | dual specificity phosphatase 9 | 0.003646724 |
| Mapk7 | mitogen activated protein kinase 7 | 0.003757431 |
| Tnfrsf1a | tumor necrosis factor receptor superfamily, member 1a | 0.00415812 |
| Prkx | protein kinase, X-linked | 0.004502912 |
| Nfkb2 | nuclear factor of kappa light polypeptide gene enhancer in B- | 0.004526246 |
| | cells 2, p49/p100 | |
| Pla2g12b | phospholipase A2, group XIIB | 0.005008582 |
| Mapk14 | mitogen activated protein kinase 14 | 0.005486382 |
| Arrb2 | arrestin, beta 2 | 0.005499632 |
| Map3k7ip2 | mitogen-activated protein kinase kinase kinase 7 interacting | 0.005572035 |
| | protein 2 | |
| Fgf14 | fibroblast growth factor 14 | 0.005893794 |
| Map2k4 | mitogen activated protein kinase kinase 4 | 0.006352425 |
| Map3k4 | mitogen activated protein kinase kinase kinase 4 | 0.006428794 |
| Rapgef4 | Rap guanine nucleotide exchange factor (GEF) 4 | 0.00646519 |
| B230120H23Rik | RIKEN cDNA B230120H23 gene | 0.007287265 |
| Fgfr1 | fibroblast growth factor receptor 1 | 0.008101899 |
| Nfatc2 | nuclear factor of activated T-cells, cytoplasmic, calcineurin- | 0.008135684 |
| | dependent 2 | |
| Mapk1 | mitogen activated protein kinase 1 | 0.009114735 |
| Casp3 | caspase 3 | 0.01030442 |
| Atf2 | activating transcription factor 2 | 0.010609392 |
| Pdgfrb | platelet derived growth factor receptor, beta polypeptide | 0.011061486 |
| Rasgrp1 | RAS guanyl releasing protein 1 | 0.01152764 |
| Pla2g2f | phospholipase A2, group IIF | 0.011765433 |
| Map3k5 | mitogen activated protein kinase kinase kinase 5 | 0.013138994 |
| Map4k4 | mitogen-activated protein kinase kinase kinase kinase 4 | 0.013794331 |
| Ntf3 | neurotrophin 3 | 0.015551899 |
| Prkacb | protein kinase, cAMP dependent, catalytic, beta | 0.015732516 |
| Fgf13 | fibroblast growth factor 13 | 0.016029769 |
| Nras | neuroblastoma ras oncogene | 0.01603981 |
| Crk | v-crk sarcoma virus CT10 oncogene homolog (avian) | 0.016282 |
| Cdc42 | cell division cycle 42 homolog (S. cerevisiae) | 0.016346284 |
| Mapk9 | mitogen activated protein kinase 9 | 0.016360449 |
| Mef2c | myocyte enhancer factor 2C | 0.016628602 |
| Ikbkb | inhibitor of kappaB kinase beta | 0.017489001 |
| Pak1 | p21 (CDKN1A)-activated kinase 1 | 0.019850033 |
| Stk3 | serine/threonine kinase 3 (Ste20, yeast homolog) | 0.020054912 |
| Pla2g4a | phospholipase A2, group IVA (cytosolic, calcium-dependent) | 0.020689473 |
| Mapk8ip3 | mitogen-activated protein kinase 8 interacting protein 3 | 0.021525545 |
| Ntrk2 | neurotrophic tyrosine kinase, receptor, type 2 | 0.023029898 |
| Map2k1 | mitogen activated protein kinase kinase 1 | 0.023250303 |
| Elk4 | ELK4, member of ETS oncogene family | 0.023453863 |
| Il1r2 | interleukin 1 receptor, type II | 0.024099017 |
| Ppm1a | protein phosphatase 1A, magnesium dependent, alpha isoform | 0.02798708 |
| Elk1 | ELK1, member of ETS oncogene family | 0.028133111 |
| Map3k12 | mitogen activated protein kinase kinase kinase 12 | 0.028611936 |
| Grb2 | growth factor receptor bound protein 2 | 0.030613258 |
| Dusp4 | dual specificity phosphatase 4 | 0.031594265 |
| Atf4 | activating transcription factor 4 | 0.032840296 |
| Ptprr | protein tyrosine phosphatase, receptor type, R | 0.034302631 |
| Map3k14 | mitogen-activated protein kinase kinase kinase 14 | 0.034493988 |
| Ppp3cb | protein phosphatase 3, catalytic subunit, beta isoform | 0.035522355 |
| Map4k3 | mitogen-activated protein kinase kinase kinase kinase 3 | 0.036013688 |
| Map4k1 | mitogen activated protein kinase kinase kinase kinase 1 | 0.037140547 |
| Mapkapk5 | MAP kinase-activated protein kinase 5 | 0.038864042 |
| Tmem37 | transmembrane protein 37 | 0.039176243 |
| Tnik | TRAF2 and NCK interacting kinase | 0.040413687 |
| Prkca | protein kinase C, alpha | 0.04096107 |
| Sos1 | Son of sevenless homolog 1 (Drosophila) | 0.041150981 |
| Hspa5 | heat shock 70 kD protein 5 (glucose-regulated protein) | 0.043896851 |
| Ntrk1 | neurotrophic tyrosine kinase, receptor, type 1 | 0.044662799 |
| Rps6ka4 | ribosomal protein S6 kinase, polypeptide 4 | 0.045486593 |
| Srf | serum response factor | 0.048518972 |
| Pdgfb | platelet derived growth factor, B polypeptide | 0.049732811 |

TABLE 6
Genes from MAPK pathways affected as defined by q < 0.05 in hearts from Lmna^{H222P/+} mice.

| Gene symbol | Gene name | q-value |
|---|---|---|
| Fgf12 | fibroblast growth factor 12 | 3.60E−06 |
| Evi1 | ecotropic viral integration site 1 | 1.08E−04 |
| Raf1 | v-raf-leukemia viral oncogene 1 | 1.11E−04 |
| Tgfbr2 | transforming growth factor, beta receptor II | 2.29E−04 |
| B230120H23Rik | RIKEN cDNA B230120H23 gene | 2.45E−04 |
| Ppm1a | protein phosphatase 1A, magnesium dependent, alpha isoform | 2.59E−04 |
| Stmn1 | stathmin 1 | 2.64E−04 |
| Mapk8 | mitogen activated protein kinase 8 | 2.69E−04 |
| Fgf10 | fibroblast growth factor 10 | 4.00E−04 |
| Rasgrp1 | RAS guanyl releasing protein 1 | 4.05E−04 |
| Map3k7ip2 | mitogen-activated protein kinase kinase kinase 7 interacting | 4.59E−04 |
| | protein 2 | |
| Pdgfra | platelet derived growth factor receptor, alpha polypeptide | 4.98E−04 |
| Rap1b | RAS related protein 1b | 6.48E−04 |
| Dusp9 | dual specificity phosphatase 9 | 6.53E−04 |
| Srf | serum response factor | 7.34E−04 |
| Flnb | filamin, beta | 8.02E−04 |
| Map3k5 | mitogen activated protein kinase kinase kinase 5 | 0.00121393 |
| Pak1 | p21 (CDKN1A)-activated kinase 1 | 0.001274338 |
| Tgfb2 | transforming growth factor, beta 2 | 0.001350584 |
| Rasa2 | RAS p21 protein activator 2 | 0.001385751 |
| Rasgrf1 | RAS protein-specific guanine nucleotide-releasing factor 1 | 0.001568889 |
| Fgfr1 | fibroblast growth factor receptor 1 | 0.001913437 |
| Mapk1 | mitogen activated protein kinase 1 | 0.001942955 |
| Tnfrsf1a | tumor necrosis factor receptor superfamily, member 1a | 0.002375645 |
| Rasa1 | RAS p21 protein activator 1 | 0.002487897 |
| Pdgfa | platelet derived growth factor, alpha | 0.002639407 |
| Stk4 | serine/threonine kinase 4 | 0.002681895 |
| Map4k3 | mitogen-activated protein kinase kinase kinase kinase 3 | 0.002754704 |
| Traf6 | Tnf receptor-associated factor 6 | 0.003022471 |
| Map2k1 | mitogen activated protein kinase kinase 1 | 0.003068013 |
| Ntrk2 | neurotrophic tyrosine kinase, receptor, type 2 | 0.003238241 |
| Arrb2 | arrestin, beta 2 | 0.003281171 |
| Map3k12 | mitogen activated protein kinase kinase kinase 12 | 0.003364804 |
| Ppp3ca | protein phosphatase 3, catalytic subunit, alpha isoform | 0.0043393 |
| Ikbkb | inhibitor of kappaB kinase beta | 0.004378057 |
| Fgf9 | fibroblast growth factor 9 | 0.004713371 |
| Tgfbr1 | transforming growth factor, beta receptor I | 0.004829289 |
| Nf1 | neurofibromatosis 1 | 0.004963115 |
| Il1r2 | interleukin 1 receptor, type II | 0.005098257 |
| Tmem37 | transmembrane protein 37 | 0.005264078 |
| Fgfr4 | fibroblast growth factor receptor 4 | 0.005404511 |
| Elk4 | ELK4, member of ETS oncogene family | 0.005409921 |
| Pla2g4a | phospholipase A2, group IVA (cytosolic, calcium-dependent) | 0.005475986 |
| Prkaca | protein kinase, cAMP dependent, catalytic, alpha | 0.005507381 |
| Dusp7 | dual specificity phosphatase 7 | 0.00564084 |
| Map2k7 | mitogen activated protein kinase kinase 7 | 0.006080179 |
| Atf4 | activating transcription factor 4 | 0.006608756 |
| Stk3 | serine/threonine kinase 3 (Ste20, yeast homolog) | 0.006703101 |
| Casp4 | caspase 4, apoptosis-related cysteine peptidase | 0.00719374 |
| Map3k7 | mitogen activated protein kinase kinase kinase 7 | 0.007929567 |
| Ptpn5 | protein tyrosine phosphatase, non-receptor type 5 | 0.008807588 |
| Map3k14 | mitogen-activated protein kinase kinase kinase 14 | 0.008979205 |
| Il1b | interleukin 1 beta | 0.009114094 |
| Egf | epidermal growth factor | 0.009371844 |
| Ikbkg | inhibitor of kappaB kinase gamma | 0.009464352 |
| Map2k6 | mitogen activated protein kinase kinase 6 | 0.009745366 |
| Map2k1ip1 | mitogen-activated protein kinase kinase 1 interacting protein 1 | 0.010073412 |
| Acvr1b | activin A receptor, type 1B | 0.010415173 |
| Map4k4 | mitogen-activated protein kinase kinase kinase kinase 4 | 0.010658562 |
| Rap1a | RAS-related protein-1a | 0.01069035 |
| Mapk9 | mitogen activated protein kinase 9 | 0.010733895 |
| Map3k7ip1 | mitogen-activated protein kinase kinase kinase 7 interacting | 0.011029738 |
| | protein 1 | |
| Map3k4 | mitogen activated protein kinase kinase kinase 4 | 0.011082817 |
| Kras | v-Ki-ras2 Kirsten rat sarcoma viral oncogene homolog | 0.011178974 |
| Casp14 | caspase 14 | 0.011191381 |
| Dusp4 | dual specificity phosphatase 4 | 0.011423174 |
| Casp1 | caspase 1 | 0.01143588 |
| Nfatc2 | nuclear factor of activated T-cells, cytoplasmic, calcineurin- | 0.013223984 |
| | dependent 2 | |
| Rapgef4 | Rap guanine nucleotide exchange factor (GEF) 4 | 0.01378897 |
| Nr4a1 | nuclear receptor subfamily 4, group A, member 1 | 0.014080154 |
| Map3k3 | mitogen activated protein kinase kinase kinase 3 | 0.014893574 |
| Hspa1l | heat shock protein 1-like | 0.015984548 |
| Ntf3 | neurotrophin 3 | 0.016139683 |
| Fgf14 | fibroblast growth factor 14 | 0.017407472 |
| Fasl | Fas ligand (TNF superfamily, member 6) | 0.018752911 |
| Mef2c | myocyte enhancer factor 2C | 0.019680604 |
| Ppp3cb | protein phosphatase 3, catalytic subunit, beta isoform | 0.020361974 |
| Fgf22 | fibroblast growth factor 22 | 0.021250778 |
| Casp6 | caspase 6 | 0.022509927 |
| Mknk1 | MAP kinase-interacting serine/threonine kinase 1 | 0.023706562 |
| Sitpec | signaling intermediate in Toll pathway-evolutionarily conserved | 0.02385116 |
| Mapk13 | mitogen activated protein kinase 13 | 0.024399541 |
| Fgfr2 | fibroblast growth factor receptor 2 | 0.024557857 |
| Chuk | conserved helix-loop-helix ubiquitous kinase | 0.025051891 |
| Casp9 | caspase 9 | 0.025852039 |
| Mknk2 | MAP kinase-interacting serine/threonine kinase 2 | 0.026408231 |
| Fgf1 | fibroblast growth factor 1 | 0.027300185 |
| Atf2 | activating transcription factor 2 | 0.027981699 |
| Ikbke | inhibitor of kappaB kinase epsilon | 0.028390773 |
| Akt3 | thymoma viral proto-oncogene 3 | 0.029365859 |
| Pla2g12b | phospholipase A2, group XIIB | 0.029772852 |
| Prkcb1 | protein kinase C, beta 1 | 0.030051395 |
| Nlk | nemo like kinase | 0.032550417 |
| Nfkb1 | nuclear factor of kappa light chain gene enhancer in B-cells 1, | 0.03400866 |
| | p105 | |
| Ntf5 | neurotrophin 5 | 0.035237338 |
| Ppm1b | protein phosphatase 1B, magnesium dependent, beta isoform | 0.035547733 |
| Pak2 | p21 (CDKN1A)-activated kinase 2 | 0.03714335 |
| Ppp3r2 | protein phosphatase 3, regulatory subunit B, alpha isoform | 0.038363453 |
| | (calcineurin B, type II) | |
| Ntrk1 | neurotrophic tyrosine kinase, receptor, type 1 | 0.039271939 |
| Mapk8ip3 | mitogen-activated protein kinase 8 interacting protein 3 | 0.039579867 |
| Daxx | Fas death domain-associated protein | 0.0396163 |
| Prkcc | protein kinase C, gamma | 0.039729139 |
| Mapkapk2 | MAP kinase-activated protein kinase 2 | 0.040014548 |
| Crk | v-crk sarcoma virus CT10 oncogene homolog (avian) | 0.040886872 |
| Nras | neuroblastoma ras oncogene | 0.042562889 |
| Pla2g6 | phospholipase A2, group VI | 0.042567554 |
| Nfatc4 | nuclear factor of activated T-cells, cytoplasmic, calcineurin- | 0.044083907 |
| | dependent 4 | |
| Fgf5 | fibroblast growth factor 5 | 0.044422804 |
| Map4k2 | mitogen activated protein kinase kinase kinase kinase 2 | 0.045341238 |
| Hspb1 | heat shock protein 1 | 0.045691236 |
| Ptprr | protein tyrosine phosphatase, receptor type, R | 0.049859977 |

Examination of MARK Inhibition on Gene Expression in Cells with LMNA Mutations

Cells transfected with plasmids encoding wild type Lamin A, Lamin A mutants that cause EDMD and other “control” Lamin A mutants that cause lipodystrophy or progeria are treated with either SP600125, PD98059, both together or vehicle (DMSO). To detect JNK and ERK activation, transfected cells are lysed in Laemmli extraction buffer (92) for subsequent immunoblotting and fixed in ice cold methanol for subsequent fluorescence microscopy. To assess activated (phosphorylated) JNK and ERK1/2 by immunoblotting, proteins in cell extracts are separated by SDS-PAGE, transferred to nitrocellulose membranes and detected using antibodies that recognize ERK1/2 (Santa-Cruz), phosphorylated ERK1/2 (Cell Signaling), JNK1 (Santa-Cruz) and phosphorylated JNK1 (Cell Signaling). Recognized proteins are visualized by enhanced chemiluminescence (ECL-Amersham). Antibodies against β-tubulin are used as an internal control to normalize the amounts of protein between blots Immunoblotting results are quantified as the ratio of signal between the protein of interest and signal of β-tubulin using Scion NIH Image software.

To assess nuclear translocation of JNK and ERK1/2, fixed cells are incubated with the same antibodies, washed, incubated with Texas Red conjugated secondary antibodies and examined by fluorescence microscopy. GFP fluorescence is simultaneously recorded to know which cells are transfected. Fluorescence microscopy is performed on a Microphot SA (Nikon) microscope attached to a Spot RT Slide camera (Diagnostic Instruments). Transiently transfected cells have been adequate for previous studies to carry out these types of experiments, but, if necessary, stably transfected cell lines expressing A-type lamins can be used, as previously described (90). To measure activation of downstream targets of activated JNK and ERK, luciferase reporter systems for c-Jun and Elk-1, respectively, are used (Path Detect In Vivo Signal Transduction Pathway Trans-Reporting System; Stratagene). Cells are cultured in the presence of SP600125, PD98059, both or vehicle and transfected with pegfp-N1 constructs encoding wild type and mutant lamins, pFA2-cJun or pFA2-Elk-1 (Stratagene) and pFR-Luc (Stratagene). To correct for transfection efficiency, a plasmid encoding β-galactosidase is co-transfected. After 24 hours, cells are trypsinized, protein lysates obtained and luciferase activity measured using a luminometer.

### Example 2

### MAP Kinase Inhibition Prevents Cardiomyopathies

### Methods

### Inhibitors

PD98059 (Calbiochem) and SP600125 (Calbiochem) were dissolved in Dimethyl Sulfoxide (DMSO, Sigma) at a concentration of 0.5 mg/ml and were delivered to a dose of 3 mg/kg/day for 5 days a week. U0126 (Cat. #662005 EMD Biosciences) and MEK1/2 (Cat. #444939 EMD Biosciences) were also dissolved in DMSO and delivered 5 days a week. The placebo control consisted of DMSO alone. Placebo and inhibitors were administered by intraperitoneal injection using a 27_{G}⁵/₈ syringe. Treatment was started when mice were 8 weeks of age and continued until 16 weeks of age.

### Mice

Lmna H222P knock-in mice were generated and genotyped as described (14). Genotyping of mice for the Lmna H222P allele was performed by PCR using oligonucleotides 5′-CAGCCATCACCTCTCCTTTG-3′ [SEQ ID NO: 2] and 5′-AGCACCAGGGAGAGGACAGG-3′ [SEQ ID NO: 3]. Lmna^{H222P/H222P} mice were separated by sex and were given either vehicle alone (DMSO), the MEK inhibitor PD98059 alone, the JNK inhibitor SP600125 alone, both PD98059 and SP600125 together, the MEK inhibitor U0126 alone, or the MEK inhibitor MEK1/2 alone. All the mice were fed on a chow diet and housed in a barrier facility. The Institutional Animal Care and Use Committee at Columbia University Medical Center approved the use of animals in the study protocol.

### Protein Extraction and Western Blots

Hearts were excised from mice at 16-weeks of age and were homogenized in RIPA extraction buffer (Cell Signalling) containing protease inhibitors (25 mg/ml aprotinin and 10 mg/ml leupeptin). Protein samples were subjected to SDS-PAGE, transferred to nitrocellulose membranes and blotted with primary antibodies against ERK1/2 (Santa-Cruz), phosphorylated ERK1/2 (Cell Signaling), JNK (Santa-Cruz), natriuretic peptide precursor A (Santa-Cruz), phosphorylated JNK (Cell Signaling), and Gapdh (Ambion). Secondary antibodies were HRP-conjugated (Amersham). Recognized proteins were visualized by enhanced chemiluminescence (ECL, Amersham). The signal generated using an antibody against Gapdh was used as internal controls to normalize the amounts of protein between immunoblots.

### RNA Isolation and Quantitative Real-Time RT-PCR Analysis

Total RNA was extracted using the Rneasy isolation kit (Qiagen) as previously described (96). cDNA was synthesized as previously described (96) using Omniscript Reverse Transcriptase (Qiagen) on total cellular RNA. For each replicate in each experiment, RNA from tissue samples of different animals was used. Primers were designed that correspond to mouse RNA sequences using Primer3 (http://frodo.wi.mit.edu/cgi-biniprimer3/primer3_www.cgi). The real-time RT-PCR reaction contained iQ SYBR green super mix (Bio-Rad), 200 nM of each primer and 0.2 μl of template in a 25 μl reaction volume. Amplification was carried out using appropriate primers and the MyiQ Single-Color Real-Time PCR Detection System (Bio-Rad) with an initial denaturation at 95° C. for 2 min followed by 50 cycles at 95° C. for 30 s and 62° C. for 30 s. Relative levels of mRNA expression were calculated using the CT method (67). Individual expression values were normalized by comparison with Gapdh mRNA.

### Pathological Analysis of Hearts

Mice were sacrificed at 16 weeks of age and freshly removed hearts were fixed in 4% formaldehyde for 48 hours, embedded in paraffin, sectioned at 5 nm and stained with hematoxylin and eosin and Masson's trichrome. Representative stained sections were photographed using a Microphot SA (Nikon) light microscope attached to a Spot RT Slide camera (Diagnostic Instruments). Images were processed using Adobe Photoshop 6.0 (Adobe Systems). Length of cardiomyocytes was measured using Scion Image software (Scion Corporation). Data were reported as means±standard deviations and are compared with respective controls using a two-tailed t test.

### Transthoracic Echocardiography

At 16 weeks of age, mice were anesthetized with 1.5% isoflurane in O₂ and placed on a heating pad (37° C.). Cardiac function was assessed by echocardiography with a Visualsonics Vevo 770 ultrasound with a 30-MHz transducer applied to the chest wall. Cardiac ventricular dimensions and ejection fraction were measured in 2D-mode and M-mode three times for the number of animals indicated. A “blinded” echocardiographer, unaware of the genotype or treatment, performed the examinations.

### Statistical Analysis

To determine significant differences between groups of animals analyzed by echocardiography, we used one-way analysis of variance (ANOVA). For each parameter, there was a global effect between different groups (p<0.001). This indicated that at least one group had significantly different results than another. We then used a Tukey adjustment for post hoc multiple comparisons (5% error type I) to determine which groups were significantly different. Homogeneity of variances between groups was validated using Levene test (alpha=0.05). Normality of residuals was validated using Shapiro-Wilk test. To validate all results, non-parametric tests (Kruskal-Wallis and Mann-Whitney) were performed and concordance checked. Other statistical methods used are described in the figure legends.

Systemic Treatment of Lmna^{H222P/H222P} Mice with PD98050 Inhibits ERK Activity in Heart

We have demonstrated previously abnormal activation of the extracellular signal-regulated kinase (ERK) branch of the mitogen-activagted protein kinase (MAPK) signaling cascade in hearts of Lmna H222P “knock in” mice, a model of autosomal Emery-Dreifuss muscular dystrophy (96). Male Lmna^{H222P/H222P} mice develop left ventricular (LV) dilatation and depressed contractile function starting at approximately 8 to 10 weeks of age and invariably develop LV dilatation and decreased cardiac contractility at 16 weeks of age, typically dying between 16 and 36 weeks (14). Based on our observations that ERK is activated in these mice prior to the onset of clinically detectable cardiomyopathy as well as our demonstration that lamin A variants that cause striated muscle disease activate ERK when expressed in cultured cells, we hypothesized that activation of ERK plays a primary pathogenic role in the development of cardiomyopathy (96).

We further hypothesized that pharmacological inhibition of ERK would prevent or delay development of dilated cardiomyopathy in Lmna^{H222P/H222P} mice. To test this hypothesis, we treated Lmna^{H222P/H222P} with compounds that inhibits MAPK/ERK kinase (MEK), thereby preventing phosphorylation (activation) of ERK (83). Here we report results of an analysis, using hearts from a mouse model of EDMD, exploring effects of an A-type lamin mutation on gene expression and signaling pathways involved in development of cardiomyopathy.

We administered PD98059, at a dose of 3 mg/kg/day or 6 mg/kg/day, or placebo (dimethylsulfoxide; DMSO) by intraperitoneal injection 5 days a week to male homozygous Lmna mutant mice (Lmna^{H222P/H222P}). PD98059 is a commercially available, potent and selective inhibitor of MEK. PD98059 mediates its inhibitory properties by binding to MEK, therefore preventing phosphorylation of ERK1/2. A comparable dose of PD98059 administered systematically has been shown to inhibit ERK activity in rat hearts (132). The doses of inhibitors used were also in the same range as those previously shown for MAPK inhibitor to be effective on the development of heart failure in the hamster (4).

Treatment was initiated at 8 weeks of age and continued until the mice were 16 weeks of age. At 16 weeks of age, the mice were analyzed by echocardiography and then sacrificed for histological and biochemical studies. Untreated male Lmna^{+/+} and Lmna^{H222P/H222P} mice were similarly analyzed for comparisons.

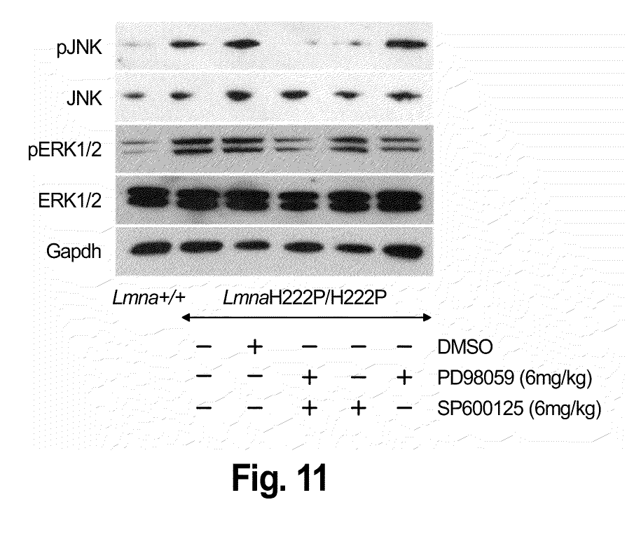

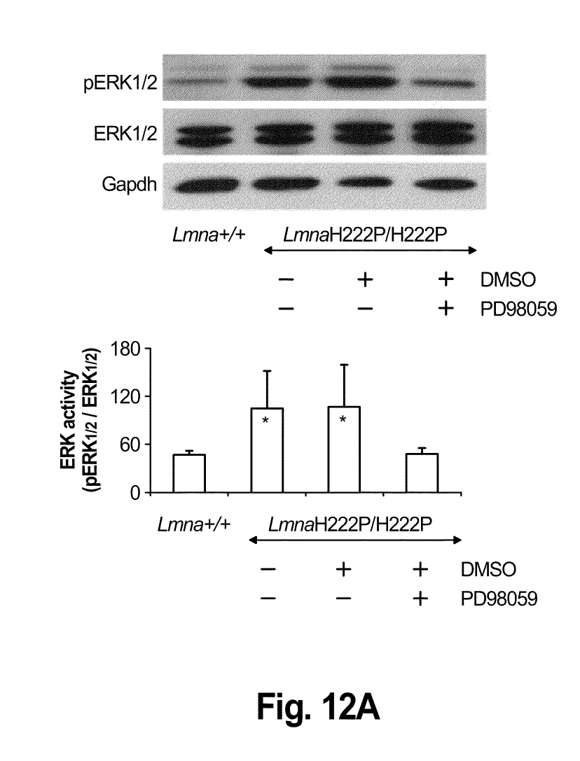

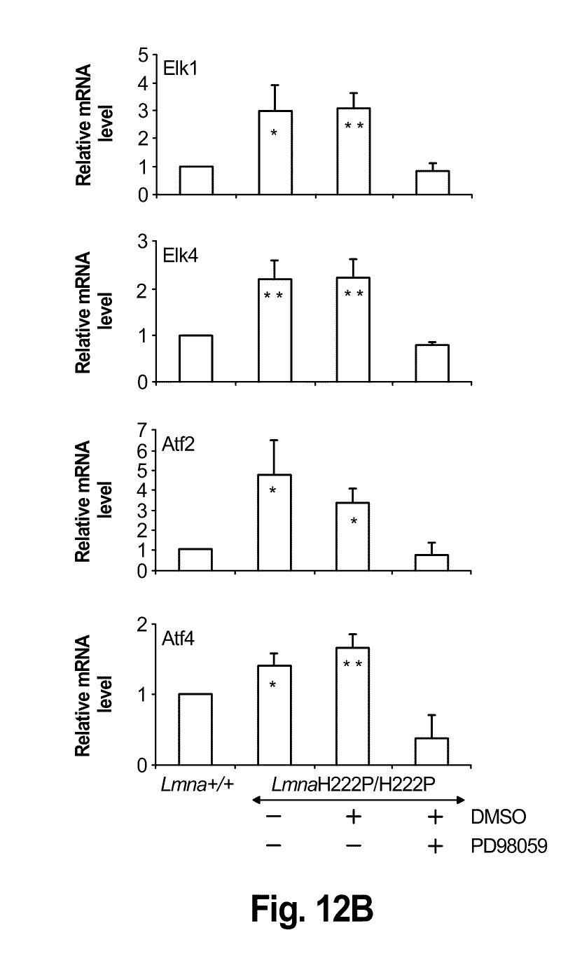

Systemic administration of PD98059 to mice inhibited phosphorylation of ERK1 and ERK2 in hearts, as shown by immunoblotting of proteins in tissue homogenates with antibodies against phoshorylated ERK1/2 and total ERK1/2 (FIGS. 11 and 12A). The inhibition was specific of ERK1/2 relative to Jun N-terminal kinase (JNK), as at a dose of 3 mg/kg/day we did not observe inhibition of JNK signaling in heart. To confirm inhibition of ERK1/2 signaling, we monitored the expression of selected downstream genes activated by the kinases using real-time RT-PCR. As expected, inhibition of phosphorylation of ERK1/2 lead to decreased expression of Elk1, Elk4, Atf2 and Atf4 (FIG. 12B).

Systemic Treatment of Lmna^{H222P/H222P} Mice with SP600125 Inhibits JNK Activity in Heart

We administered a JNK inhibitor, SP600125, to male Lmna^{H222P/H222P} mice as described above. SP600125 is a commercially available inhibitor of JNK. SP600125 blocked the phosphorylation of JNK but did not block the phosphorylation of ERK1/2. We also administered the PD98059 and SP600125 inhibitors together. SP600125 inhibits the phosphorylation of its targets in heart when administered systemically to mice, as shown by western blots of phosphorylated JNK and total JNK (FIG. 11).

Treatment with PD98050 and/or SP600125 Prevents Development of Cardiomyopathy

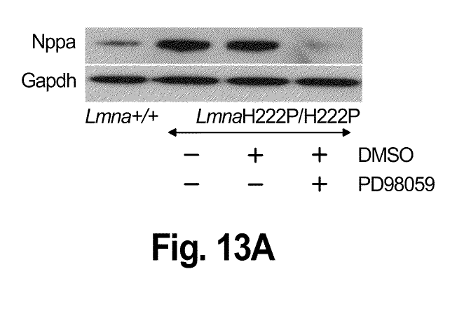

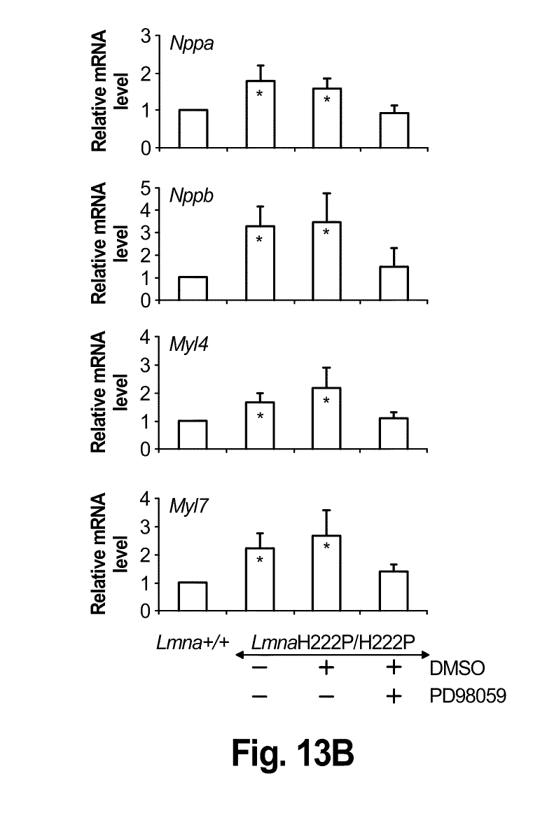

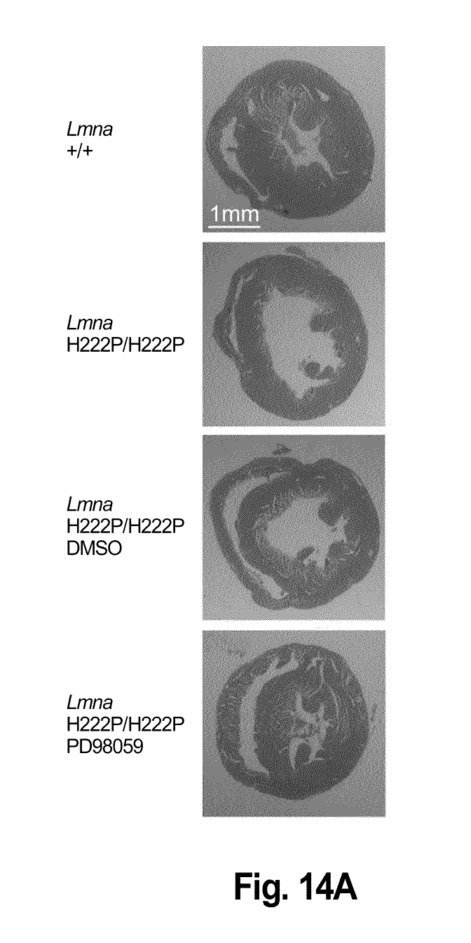

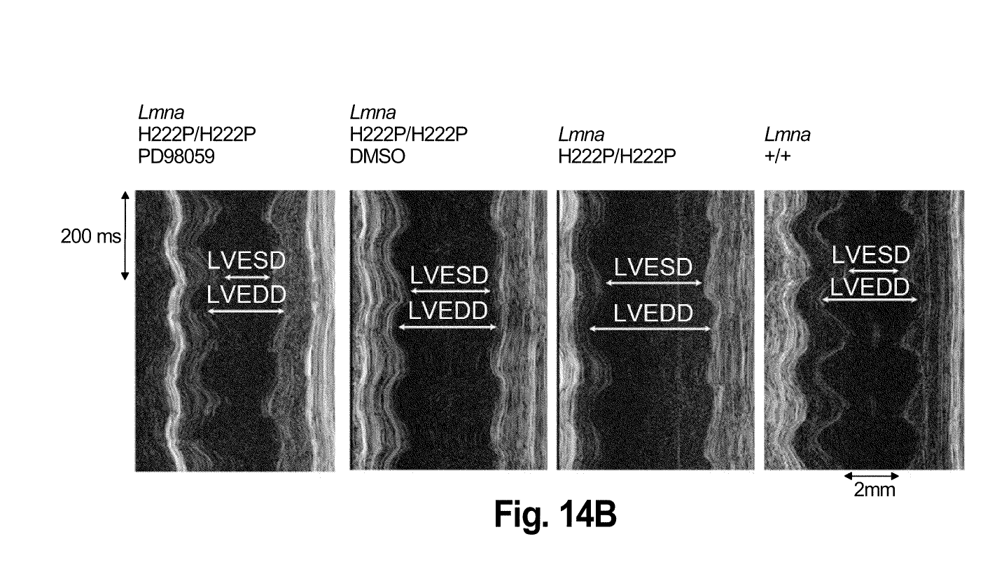

A feature of dilated cardiomyopathy is the up-regulation of cardiac hormones such as natriuretic peptides (17, 55, 133). Up-regulation of genes involved in sarcomere organization also occurs in dilated cardiomyopathies (18, 55, 134). In hearts from untreated Lmna^{H222P/H222P} mice and those treated with placebo, expression of natriuretic peptide precursor A was significantly increased (FIG. 13A). In contrast, PD98059-treated Lmna^{H222P/H222P} mice had a cardiac expression of this peptide similar to Lmna^{+/+} mice (FIG. 13A). In hearts from untreated Lmna^{H222P/H222P} mice and Lmna^{H222P/H222P} mice treated with placebo, expression of Nppa and Nppb mRNAs encoding natriuretic peptide precursors as well as Myl4 and Myl7 mRNAs encoding myosin light chains was significantly increased (FIG. 13B). In contrast, PD98059-treated Lmna^{H222P/H222P} mice had a cardiac expression of Nppa, Nppb, Myl4 and Myl7 similar to Lmna^{+/+} mice (FIG. 13B).

Lmna^{H222P/H222P} mice invariably develop dilated cardiomyopathy by 12 weeks of age. We monitored dilation as well as dynamic of the left ventricle in absence or presence of PD98059, SP600125 or both PD98059 and SP600125 in Lmna^{H222P/H222P} mice. At 16 weeks, Lmna^{H222P/H222P} ice developed left ventricle (LV) dilation. LV dilatation in male Lmna^{H222P/H222P} ice at 16 weeks of age was demonstrated by histopathological analysis (FIG. 14A). At this age, there was no significant cardiomyocyte disarray or cardiac fibrosis on light microscopic examination.

As would be measured to confirm cardiomyopathy in humans, we used M-mode transthoracic echocardiography to measure three important parameters of dilation of the LV: left ventricle end systolic diameter (LVESD) and left ventricle end diastolic diameter (LVEDD), the ejection fraction (EF), and the fractional shortening (FS). We also measured thickness of the left ventricular posterior wall (LVPW) and the interventricular septum diameter (IVSD). Treatment with PD98059 prevented development of LV dilatation as measured by histopathology and echocardiography (FIG. 14). Treatment with SP600125 prevented development of LV dilatation as measured by echocardiography (Table 7). Lmna^{H222P/H222P} mice showed increased left ventricular end systolic and end diastolic diameters compared to Lmna^{+/+} mice (FIG. 14B).

Cardiac and structure and function were further assessed by echocardiography at 16 weeks of age in a total of 43 living mice in the 5 different groups studied (Table 7). Compared to Lmna^{-/+} mice, Lmna^{H222P/H222P} mice had significantly increased LV end diastolic and end systolic diameters. They also had decreased cardiac contractility indicated by reduced ejection fraction and LV fractional shortening. Ejection fraction in Lmna^{H222P/H222P} mice was decreased by approximately 30% compared to Lmna^{+/+} mice at 16 weeks age (73.12±6.69 percent vs. 50.78±9.12 percent; p<0.005). Lmna^{H222P/H222P} mice treated with DMSO had ventricular chamber diameters, ejection fraction and LV fractional shortening similar to untreated Lmna^{H222P/H222P} mice. Lmna^{H222P/H222P} mice treated with PD98059 had normal cardiac contractility with ejection fraction and LV fractional shortening virtually identical to Lmna^{+/+} mice. With 100% accuracy in real-time, a “blinded” echocardiographer unaware of the genotype or treatment received classified all Lmna^{+/+} mice and Lmna^{H222P/H222P} mice receiving PD98059 as having normal cardiac function and all Lmna^{H222P/H222P} mice that were untreated or treated with placebo as having abnormal cardiac function. Hence, treatment with PD98059 or SP600125 for 8 weeks prevented the development of LV dilatation and cardiac contractile dysfunction in Lmna^{H222P/H222P} mice.

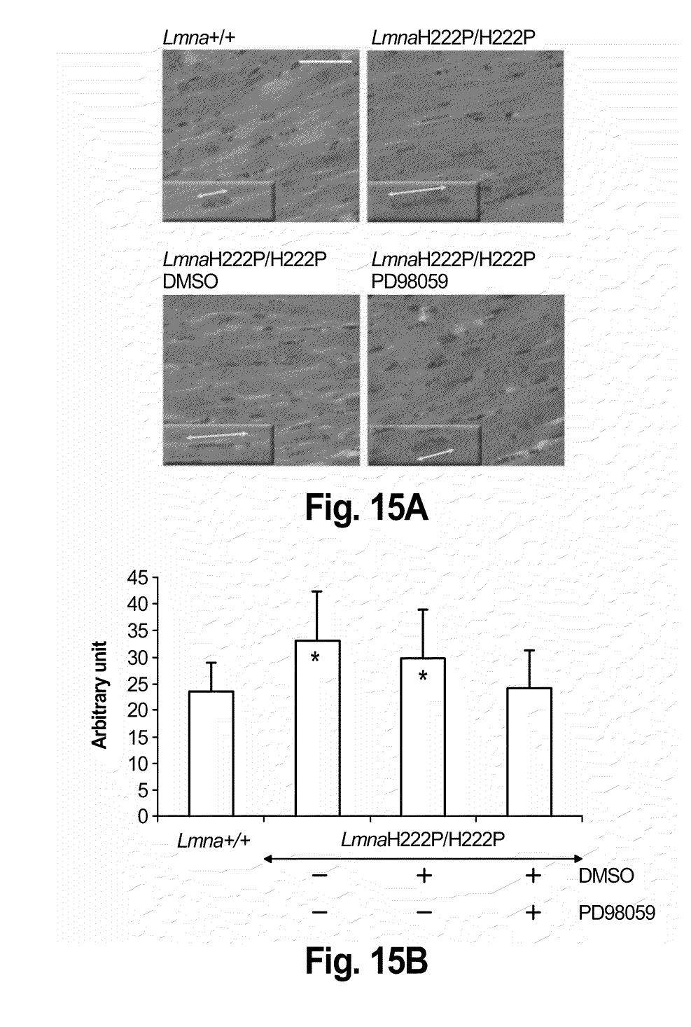

Alterations in nuclear morphology, including abnormal elongation of nuclei, have been described in hearts of mice deficient in A-type lamins that develop dilated cardiomyopathy (108). We observed similar elongation of nuclei in cardiomyocytes of 16 week-old Lmna^{H222P/H222P} mice. Treatment with PD98059 prevented this alteration. Nuclei in cardiomyocytes of Lmna^{+/+} mice had a well-rounded oval shape whereas nuclei in cardiomyocytes of Lmna^{H222P/H222P} mice had an abnormally elongated shape (FIG. 15A). Cardiomyocyte nuclei in Lmna^{H222P/H222P} mice treated with PD98059 but not placebo had an overall shape that was similar to those in Lmna^{+/+} mice (FIG. 15A). Means lengths of cardiomyocyte nuclei in untreated and placebo-treated Lmna^{H222P/H222P} mice were significantly longer than in Lmna^{+/+} mice and Lmna^{H222P/H222P} mice treated with PD98059 (FIG. 15B).

Overall, LV diameters, cardiomyocyte nuclear morphology and cardiac ejection fraction were normal in Lmna^{H222P/H222P} mice treated with PD98059 at an age when untreated and placebo-treated mice had significant abnormalities in these parameters. Enhanced synthesis of natriuretic peptides and sarcomeric proteins was also prevented. Treatment with an inhibitor of ERK activation therefore delayed the development of significant cardiomyopathy in mice with an Lmna mutation that causes Emery-Dreifuss muscular dystrophy in humans.

TABLE 7
Echocardiographic data at 16 weeks of age for LMNA^{+/+} (WT) mice and Lmna^{H222P/H222P} mice

| | | LVEDD | LVESD | LVPW | IVSD | | |
|---|---|---|---|---|---|---|---|
| Genotype | n | (mm) | (mm) | (mm) | (mm) | EF (%) | FS (%) |
| Lmna^{+/+} | 12 | 3.45 ± 0.42 | 2.00 ± 0.36 | 0.71 ± 0.10 | 0.70 ± 0.13 | 73.12 ± 6.69 | 41.72 ± 5.76 |
| Lmna^{H222P/H222P} | 6 | 4.14 ± 0.27## | 3.25 ± 0.45## | 0.82 ± 0.12 | 0.70 ± 0.05 | 50.78 ± 9.12## | 25.82 ± 5.70## |
| (mock) | | | | | | | |
| Lmna^{H222P/H222P} | 5 | 3.89 ± 0.14# | 3.04 ± 0.32## | 0.75 ± 0.08 | 0.69 ± 0.02 | 52.70 ± 9.03## | 26.96 ± 5.68## |
| (DMSO) | | | | | | | |
| Lmna^{H222P/H222P} | 7 | 3.12 ± 0.20** | 1.82 ± 0.16** | 0.77 ± 0.10 | 0.69 ± 0.06 | 73.52 ± 4.68** | 41.55 ± 4.29** |
| (PD98059) | | | | | | | |
| Lmna^{H222P/H222P} | 13 | 3.46 ± 0.27** | 2.30 ± 0.45** | 0.73 ± 0.08 | 0.72 ± 0.07 | 69.24 ± 9.59** | 38.70 ± 7.38** |
| (SP600125) | | | | | | | |
| Lmna^{H222P/H222P} | 7 | 3.65 ± 0.35 | 2.43 ± 0.37# | | | 71.13 ± 6.66 | 38.33 ± 7.24 |
| (PD98059 + | | | | | | | |
| SP600125) | | | | | | | |
| Lmna^{H222P/H222P} | 3 | 3.54 ± 0.16 | 2.47 ± 0.09 | 0.69 ± 0.11 | 0.64 ± 0.03 | 62.83 ± 4.09 | 33.79 ± 3.10 |
| (U0126)§ | | | | | | | |
| Lmna^{H222P/H222P} | 2 | 3.77 ± 0.32 | 2.62 ± 0.28 | 0.69 ± 0.08 | 0.60 ± 0.00 | 64.43 ± 0.77 | 34.50 ± 0.36 |
| (MEK1/2)§ | | | | | | | |

LVEDD: left ventricle end diastolic diameter; LVESD: left ventricle end systolic diameter; LVPW: left ventricular posterior wall; IVSD: interventricular septum diameter; EF: ejection fraction; FS: fractional shortening.
“Mock” indicates untreated Lmna^{H222P/H222P} mice. “DMSO” indicates Lmna^{H222P/H222P} mice treated only with vehicle.
Values are means ± standard deviations.
*p < 0.05 versus Lmna^{H222P/H222P} (DMSO);
**p < 0.01 versus Lmna^{H222P/H222P} (DMSO)
#p < 0.05 versus Lmna^{+/+};
##p < 0.01 versus Lmna^{+/+}
§Statistical analysis is not provided for U0126 and MEK1/2 due to the small n.

Our results support the hypothesis that ERK and JNK activation induced by abnormalities in A-type lamins is a pathogenic mechanism in the generation of cardiomyopathy. For example, we demonstrated inhibition of ERK phosphorylation and attenuated activation of downstream genes when PD98059 was administered systemically to Lmna^{H222P/H222P} mice. Concurrent with this inactivation of ERK signaling in heart we documented normal LV diameters, normal cardiomyocyte nuclear morphology and normal cardiac ejection fraction in Lmna^{H222P/H222P} mice treated with PD98059 at an age when untreated and placebo-treated mice had significant abnormalities in these parameters. These results are consistent with our findings that ERK is abnormally activated in cardiomyocytes of Lmna^{H222P/H222P} mice and cells expressing lamin A variants found in human subjects with cardiomypathy (96). Results from Favreau et al. (135) using cultured myoblasts also suggest that the nuclear lamina may serve as scaffold for substrates of the MEK-ERK pathway and that this may be impeded by A-type lamin alterations resulting from LMNA mutations that cause Emery-Dreifuss muscular dystrophy.

### Example 3

### MAP Kinase Inhibition Improves Cardiac Function in Existing Cardiomyopathies

Using the methods described in Example 2, we treated Lmna^{H222P/H222P} mice with PD98059 alone or SP600125 alone, starting at 16 weeks of age, when ejection fraction has already deteriorated and left ventricular end diastolic diameter is increased, until 20 weeks of age. Treatment with each of these prevented further deterioration in cardiac function (Table 8), suggesting that JNK and ERK inhibition can prevent further deterioration in cardiac function once clinically apparent cardiomyopathy is present.

TABLE 8
Echocardiographic data at 20 weeks of age for Lmna^{+/+} (WT) mice and Lmna^{H222P/H222P} mice

| | | LVEDD | LVESD | LVPW | IVSD | | |
|---|---|---|---|---|---|---|---|
| Genotype | n | (mm) | (mm) | (mm) | (mm) | EF (%) | FS (%) |
| Lmna^{+/+} | 12 | 3.50 ± 0.22 | 2.07 ± 0.28 | 0.81 ± 0.10 | 0.77 ± 0.07 | 73.21 ± 4.06 | 41.71 ± 3.50 |
| Lmna^{H222P/H222P} | 7 | 4.43 ± 0.79## | 3.80 ± 1.06## | 0.67 ± 0.09 | 0.62 ± 0.06 | 43.82 ± 20.41## | 22.49 ± 12.08## |
| (mock) | | | | | | | |
| Lmna^{H222P/H222P} | 22 | 3.87 ± 0.50# | 3.00 ± 0.61## | 0.64 ± 0.13 | 0.63 ± 0.11 | 53.87 ± 12.12## | 27.86 ± 7.22## |
| (DMSO) | | | | | | | |
| Lmna^{H222P/H222P} | 19 | 3.55 ± 0.50 | 2.41 ± 0.50**# | 0.69 ± 0.08 | 0.66 ± 0.05 | 65.46 ± 11.49**# | 35.91 ± 8.21** |
| (PD98059) | | | | | | | |
| Lmna^{H222P/H222P} | 26 | 3.73 ± 0.42 | 2.67 ± 0.49*## | 0.66 ± 0.09 | 0.63 ± 0.07 | 61.88 ± 8.46**## | 33.11 ± 5.92**## |
| (SP600125) | | | | | | | |

LVEDD: left ventricle end diastolic diameter; LVESD: left ventricle end systolic diameter; LVPW: left ventricular posterior wall; IVSD: interventricular septum diameter; EF: ejection fraction; FS: fractional shortening.
Comparison between groups was performed using one-way ANOVA and Tukey adjustment for post hoc multiple comparison (5% type I error). Conditions of homogeneity of variances were validated and non-parametric tests were performed to validate results.
Values are means ± standard deviations.
*p < 0.05 versus Lmna^{H222P/H222P} (DMSO);
**p < 0.01 versus Lmna^{H222P/H222P} (DMSO)
#p < 0.05 versus Lmna^{+/+};
##p < 0.01 versus Lmna^{+/+}

### Further Clinical Assessment of Knock-in Mice

We examine effects of JNK and ERK inhibitors on heart function and survival in Lmna^{H222P/H222P} mice. Male Lmna^{H222P/H222P} and Lmna^{+/+} mice are treated with SP600125, PD98059, both or placebo as described above. We begin treatment at 8 weeks of age. Mice are assessed by electrocardiography prior to starting treatment and at 2 weeks, 8 weeks, 16 weeks and 24 weeks after treatment (10 weeks, 16 weeks, 24 weeks and 32 weeks of age, respectively). Ages of 10, 16 and 24 weeks correspond to those at which MAP kinase activities are assessed. Blood is drawn at these times for analysis of complete blood count, routine chemistries and cardiac enzymes. Male Lmna^{H222P/H222P} mice typically begin to develop abnormalities detected by echocardiography starting at 8 weeks of age and conduction system abnormalities, primarily an increased PR interval, at 12 weeks of age. If Lmna^{H222P/H222P} mice survive beyond 32 weeks of age (medial survival is 28 weeks), analyses continue to be performed at 2 to 4 week intervals in these mice and Lmna+/+ controls. Kaplan-Meier analysis (95) is performed to compare survival between groups. For electrocardiography, transmitters are placed in the abdominal region under anesthesia with ketamine, xylazine and midazolam. Signals are sent to a computer for display and analysis. Telemetric electrocardiography tracings are also obtained in conscious mice during quiet awake time at daytime, and PR intervals and QRS durations are measured.

Results demonstrating that MAP kinase inhibition provides a clinical benefit in an animal model of autosomal dominant EDMD pave the way for testing in human subjects and provides a model for drug discovery for the treatment of neuromuscular disorders in which the pathogenesis is unclear.

### Example 4

### Reduced Expression of A-type Lamins and Emerin Activates ERK Signaling Pathway in Cell Lines

Mutations in genes encoding ubiquitously expressed A-type lamins and emerin, proteins of the nuclear envelope, respectively cause autosomal and X-linked Emery-Dreifuss muscular dystrophy (EDMD), which affects skeletal and cardiac muscles. We identified an activation of extracellular signal-regulated kinase (ERK), a branch of the MAP kinase signaling pathway, in hearts from mouse models of these two forms of EDMD, which could explain the pathogenesis of the disease. To examine the relation between the nuclear envelope and the activation of ERK, we studied the effect of decreasing A-type lamins and emerin using model mice and siRNA technology.

Loss of A-type lamins in mouse heart leads to the activation of ERK. We showed that knock-down of A-type lamins and emerin in HeLa and C2C12 cells using siRNA duplexes induced phosphorylation of ERK and activation of downstream transcription factors. The use of a specific MAPK/ERK kinase (MEK) inhibitor abolished this abnormal activation.

Our results further demonstrate that abnormalities in the expression of nuclear envelope proteins lead to activation of ERK signaling. This has implications for pharmacological therapy of EDMD.

### Methods

### Mice

Lmna^{-/-} mice were generated and genotyped as described [11]. Hearts were isolated from male Lmna^{-/-} and Lmna^{+/+} mice at 5 weeks of age. For immunoblotting and real-time RT-PCR experiments, Lmna^{-/—} and Lmna^{+/+} mice were compared directly to Lmna^{+/+} littermates.

### Cell Culture

Human HeLa cells and mouse C2C12 cells were maintained in a 5% CO₂ atmosphere at 37° C. The cells were cultured in Dulbecco's modified Eagle's medium supplemented with 10% calf bovine serum and 0.1% gentamicin.

siRNA

One day before transfection, HeLa and C2C12 cells were trypsinized, diluted with fresh medium without antibiotics and transferred to 24-well plates. Transient transfection of siRNAs was carried out using Oligofectamine (Invitrogen) as recommended by the manufacturer. Cells were preincubated in 7.5 μl OPTIMEM 1 medium (Life Technologies) and 2 μl Oligofectamine per well for 5 minutes at room temperature. During the time of this incubation, 40 μl OPTIMEM 1 medium were mixed with 2.5 μl siRNA. The two mixtures were combined and incubated for 20 minutes at room temperature for complex formation. The entire mixture was added to the cells in one well resulting in a final concentration of 50 pM for the siRNAs. Cells were assayed 72 hours after transfection for HeLa cells and 48 hours after transfection for C2C12 cells. Reduction of expression of targeted genes was confirmed in at least 3 independent experiments.

### RNA Extraction

At 80% confluence, media was removed from cultures and total RNA was extracted using the Rneasy isolation kit (Qiagen) according to the manufacturer's instructions. Adequacy and integrity of extracted RNA were determined by gel electrophoresis. Concentrations were measured by ultraviolet absorbance spectroscopy.

### Indirect Immunofluorescence Microscopy

HeLa and C2C12 cells were grown on coverslips and washed with phosphate-buffered saline (PBS). Cells were fixed for 10 minutes in methanol at −20° C. HeLa and C2C12 cells were then incubated with the primary antibody in PBS for 1 hour at room temperature. Primary antibody used was anti-pERK polyclonal (1:100, Cell Signaling). Cells were then washed with PBS and incubated with Texas Red conjugated goat anti-rabbit secondary antibody in PBS (Molecular Probes). Cells were washed with PBS and slides mounted in Mowiol (Santa-Cruz Biotechnologies) with 0.1 μg/m; 4′,6-diamidino-2-phenylindole (dapi) Immunofluorescence microscopy was performed using an Axiophot microscope (Carl Zeiss). Micrographs were processed using Adobe Photoshop 6.0 (Adobe Systems).

### Real-Time RT-PCR

Primers were designed corresponding to RNA sequences using Primer3 (http://frodo.wi.mit.edu/cgi-bin/primer3/primer3_www.cgi). RNA was extracted using Rneasy Protect Kit (Qiagen) and subsequently reverse transcribed using SuperScript First-Strand Synthesis System according to the manufacturer's instructions (Invitrogen). Each reaction contained iQ SYBR green super mix (Bio-Rad), 200 nM of primers and 0.2 μl of template in a 25 μl reaction volume. Amplification was carried out using the MyiQ Single-Color Real-Time PCR Detection System (Bio-Rad) with incubation times of 2 minutes at 95° C., followed by 50 cycles of 95° C. for 30 seconds and 62° C. for 30 seconds. Specificity of the amplification was checked by melting-curve analysis. Relative levels of mRNA expression were calculated according to the ΔΔC_{T} method, normalized by comparison to Gapdh mRNA expression.

### Western Blot Analysis

HeLa and C2C12 cells were harvested from each culture, washed with ice-cold PBS and total protein extracted in buffer (25 mM Tris [pH 7.4], 150 mM NaCl, 5 mM EDTA, 10 mM sodium pyrophosphate, 1 mM Na₃VO₄, 1% SDS, 1 mM dithiothreitol) containing protease inhibitors (25 mg/ml aprotinin and 10 mg/ml leupeptin). Proteins were separated by SDS-PAGE, transferred to nitrocellulose membranes and blotted with primary antibodies against ERK1/2 (Santa-Cruz), pERK1/2 (Cell Signaling), lamin A/C (Santa-Cruz), emerin (Novocatra), β-actin (Sant-Cruz) and Gapdh (Santa-Cruz). Secondary antibodies were HRP-conjugated (Amersham). Recognized proteins were visualized by enhanced chemiluminescence (ECL-Amersham) and visualized using Hyperfilm ECL (Amersham). The signal generated using antibody against β-actin was used as an internal control to normalize the amounts of protein between immunoblots. Band densities were calculated using Scion Image software (Scion Corporation) and normalized to the appropriate total extract to control for protein loading. Data are reported as means±standard deviations and are compared with respective controls using a two-tailed t test.

### Colorimetric Analysis of ERK1/2 Phosphorylation

HeLa and C2C12 cells were cultured for 24 hours in the presence of PD98059 (45 μM). ERK1/2 phosphorylation was measured using an Enzyme-Linked ImmunoSorbent Assay (ELISA) (SuperArray CASE, ERK1/2 kit) as per the manufacturer's protocol. Briefly, cells were fixed and stained with either phospho-ERK1/2 or ERK1/2 primary antibodies (1 hour at room temperature). After a wash and incubation with secondary antibody (1 hour at room temperature), cells were incubated with color developer (10 minutes at room temperature) and plates were read at an optical density (OD) of 450 nm. Thereafter, relative cell number was assayed in each well (OD of 595 nm) to normalize the antibody reading. To determine the ERK1/2 phosphorylation, we normalized the phospho-ERK1/2 signal ratio (OD_{450 nm}/OD_{595 nm}) to the total ERK1/2 signal ratio (OD_{450 nm}/OD_{595 nm}). Data are reported as means±standard deviations and are compared with respective controls using a two-tailed t test.

Activation of ERK1/2 in Hearts from Mice Without A-Type Lamins

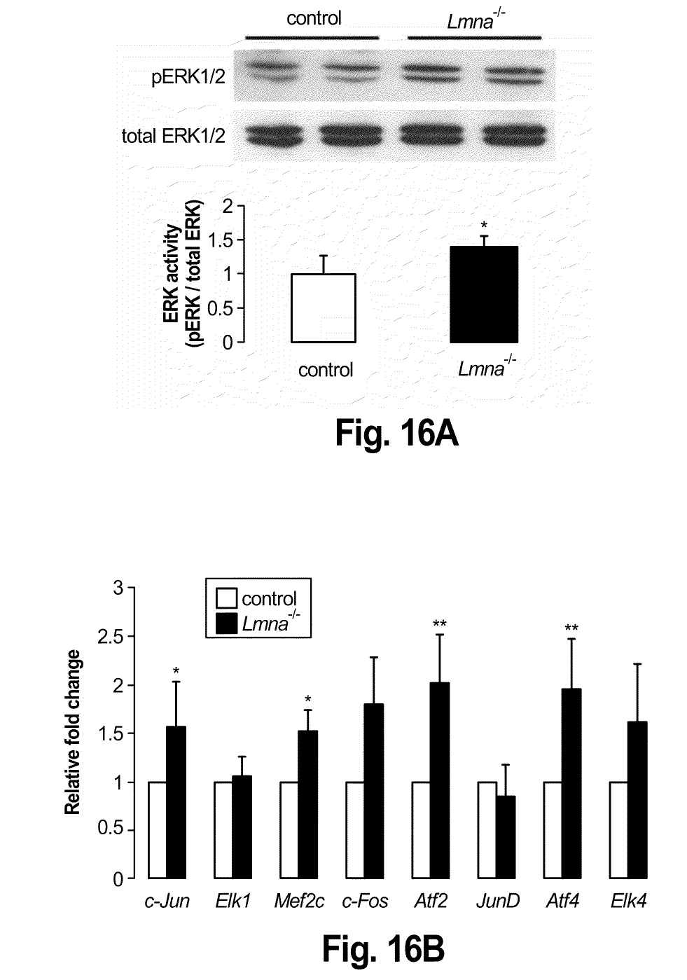

We have previously shown that a point mutation in Lmna that causes EDMD and loss of emerin both induce activation of ERK, one of the branches of MAP kinase signaling pathway, in hearts of mice prior to cardiac dysfunction [69, 89]. Loss of A-type lamins in mice has been described previously as leading to a muscular dystrophy and cardiomyopathy [101]. To examine if loss of A-type lamins induces an aberrant activation of ERK1/2, we analyzed expression of the phosphorylated ERK1/2 in hearts from mice 5 weeks of age. Phosphorylated ERK1/2 was activated 1.4±0.17 fold in hearts from Lmna^{-/-} mice compared to hearts from control mice when analyzed by immunoblotting (FIG. 16A). We also analyzed the expression of genes activated downstream in the ERK1/2 signaling pathway by real-time quantitative RT-PCR. There was significantly increased expression of c-Jun, Mef2c, Atf2 and Atf4 in hearts of Lmna^{-/-} mice compared to Lmna^{+/+} control mice (FIG. 16B). These results showed an activation of ERK pathway in hearts of Lmna^{-/-} mice.

Targeted Knockdown of Emd and Lmna Genes Using siRNAs

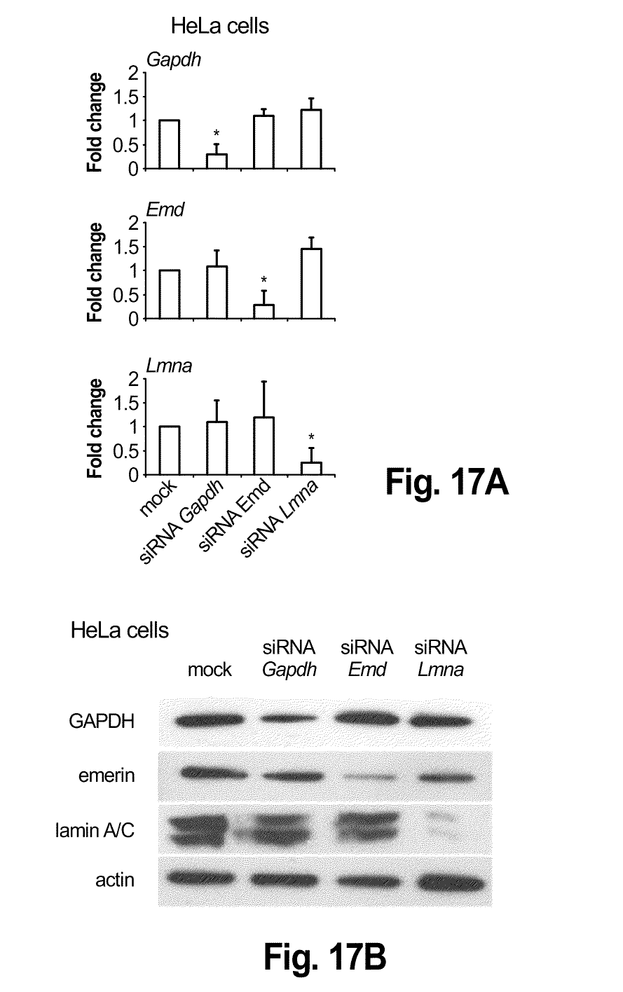

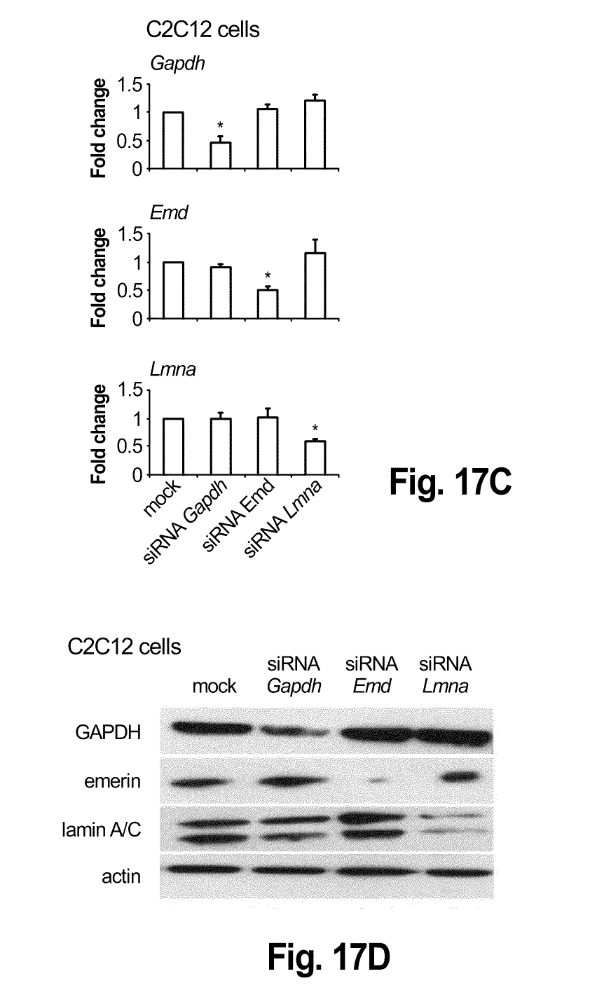

To further investigate the role of nuclear envelope proteins in activation of ERK signaling, we used a human cell line (HeLa cells) and a mouse myogenic cell line (C2C12 cells) and knocked down targeted genes using siRNA technology. After a 72 hours treatment for HeLa cells, total RNA and proteins were extracted from cells cultured without siRNA treatment (mock) and from cells cultured with Gapdh, Emd and Lmna siRNAs. When Gapdh, Emd and Lmna siRNAs were transfected into HeLa cells, the corresponding mRNAs (FIG. 17A) and proteins (FIG. 17B) were reduced of approximately 50%. In C2C12 cells, total RNA and proteins were extracted after 48 hours treatment in mock treated cells and in cells cultured with Gapdh, Emd and Lmna siRNA duplexes. When Gapdh, Emd and Lmna siRNAs were transfected in C2C12 cells, the corresponding mRNAs (FIG. 17C) and proteins (FIG. 17D) were markedly reduced of 50%. These observations demonstrated that treatment with siRNAs was successful to partially reduce the targeted mRNAs.

Activation of ERK Signaling Pathway in Cells with Knocked Down A-Type Lamins or Emerin

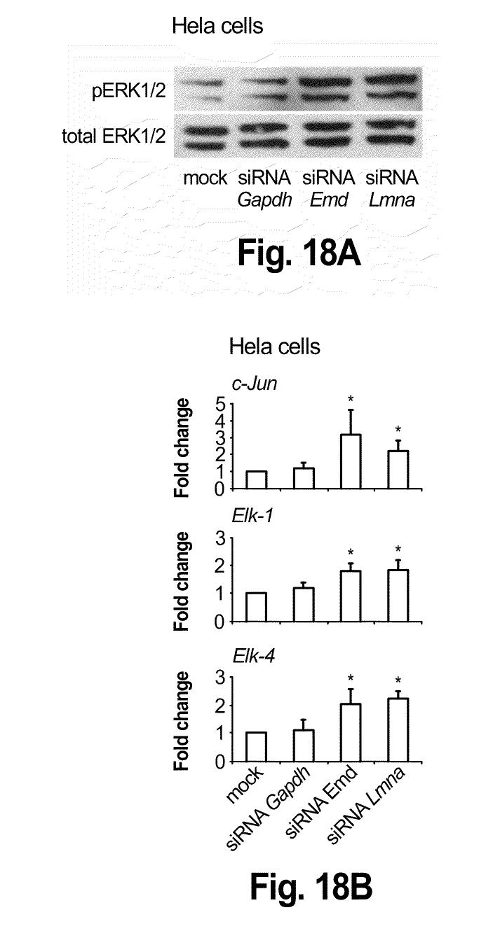

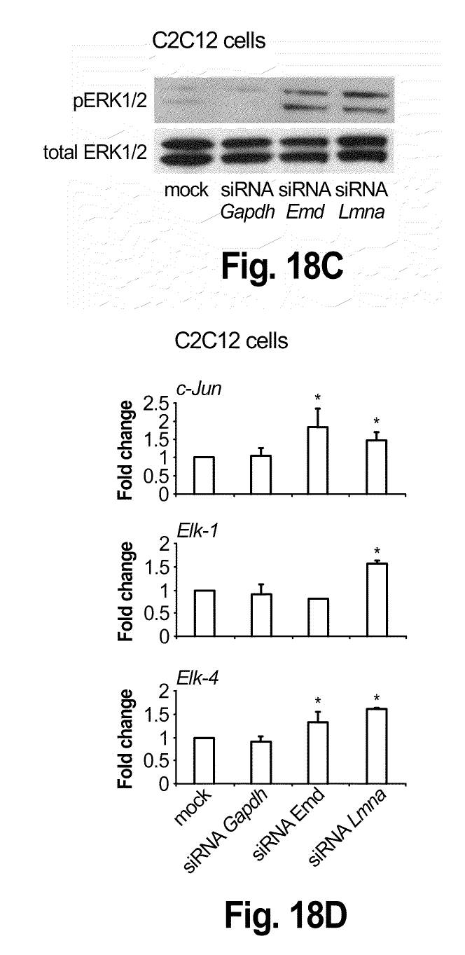

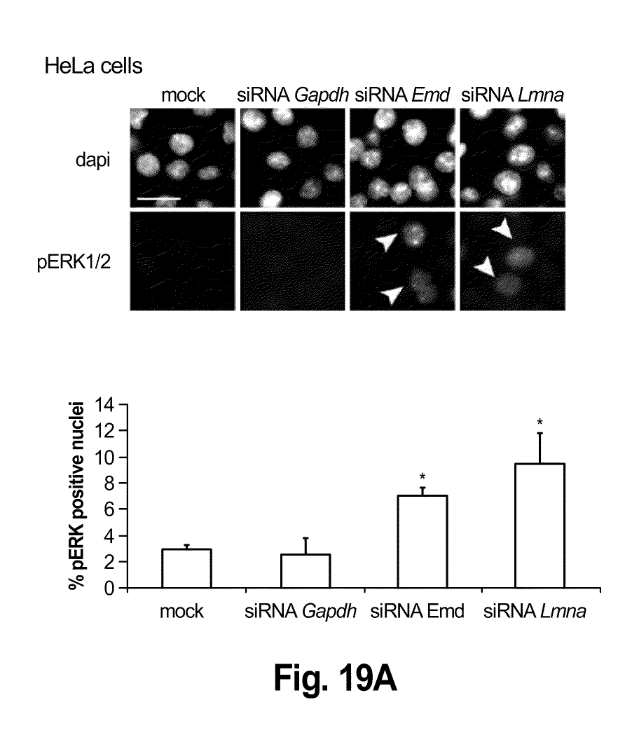

To determine if treatment with siRNAs in HeLa and C2C12 cells lead to activation of ERK signaling pathway, we first evaluated phosphorylation of ERK1/2 Immunoblotting with anti-pERK1/2 antibody demonstrated an increase in pERK1/2 in HeLa cells treated with Emd and Lmna siRNA duplexes whereas no significant increase was observed in mock treated cells or cells treated with GAPDH siRNA duplex (FIG. 18A). Phosphorylated ERK1/2 activates a series of downstream target genes, including those encoding c-Jun, Elk1 and Elk4. We analyzed the expression of these transcripts using real-time quantitative RT-PCR. While these individual genes were not found to be significantly differentially expressed in mock treated cells and cells treated with Gapdh siRNA (FIG. 18B), treatment with Emd and Lmna siRNAs lead to enhanced expression of c-Jun, Elk1 and Elk4 (FIG. 18B). Abnormal activation of pERK1/2 was also observed in C2C12 treated with Emd and Lmna siRNAs (FIG. 18C). We also analyzed the expression of downstream target genes and found an aberrant up-regulation of c-Jun and Elk4 when C2C12 cells were treated with Emd and Lmna siRNAs (FIG. 18D). The expression of Elk1 was increased only in C2C12 cells treated with Lmna siRNA (FIG. 18D).

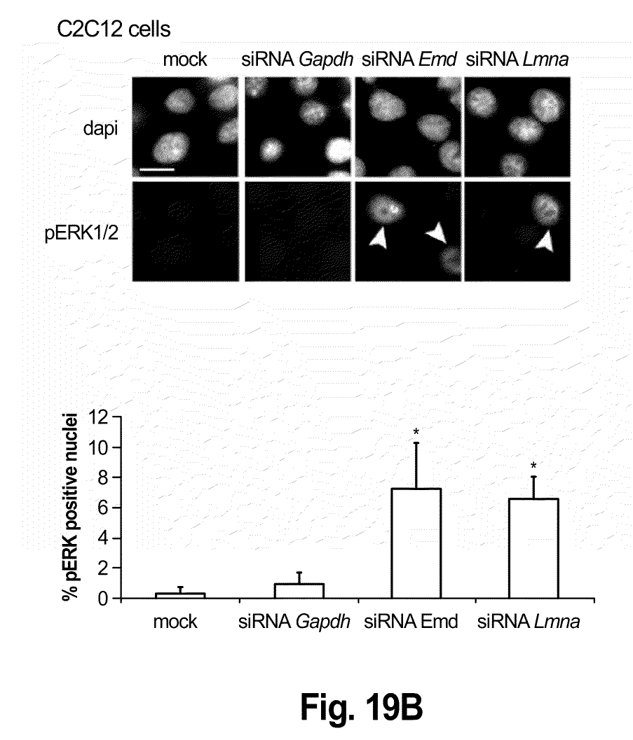

Translocation of pERK1/2 from cytoplasm to nucleus is necessary for activation of downstream genes. In mock treated HeLa cells and HeLa cells treated with Gapdh siRNA, pERK was weakly or not detectable and only approximately 2% of HeLa cells showed a nuclear localization of pERK (FIG. 19A). In contrast, treatment with Emd and Lmna siRNAs induced translocation of pERK into the nucleus in significantly more cells (FIG. 19A, arrowheads). Approximately 8% of HeLa cells treated with Emd siRNA and 10% of HeLa cells treated with Lmna siRNA showed a nuclear localization of pERK (FIG. 4A). In mock treated C2C12 cells and C2C12 cells treated with Gapdh siRNA, the activated pERK was detectable in less than 1% of nuclei (FIG. 19B). When C2C12 cells were treated with Emd or Lmna siRNAs, there was a significant increase of cells with a nuclear localization of pERK (FIG. 19B, arrowheads). Approximately 6% of C2C12 cells treated with Emd or Lmna siRNAs showed an intranuclear localization of pERK (FIG. 19B). Hence, knocking down expression of Emd and Lmna induces phosphorylation and translocation of ERK and subsequent activation of downstream targets.

### ERK1/2 Activity is Decreased by a MAPK/ERK Kinase (MEK) Inhibitor in HeLa Cells Knocked Down for A-Type Lamins or Emerin

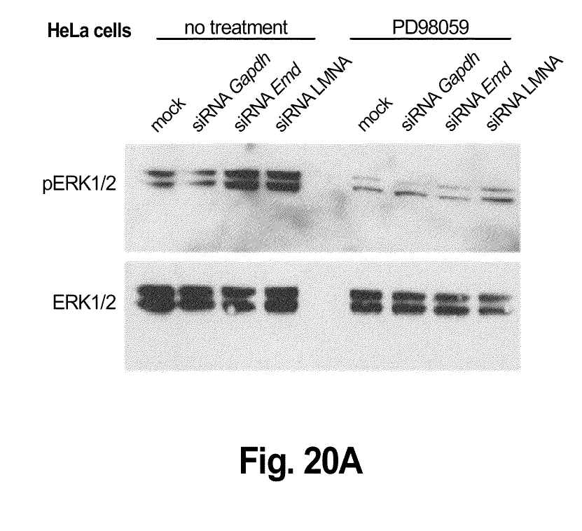

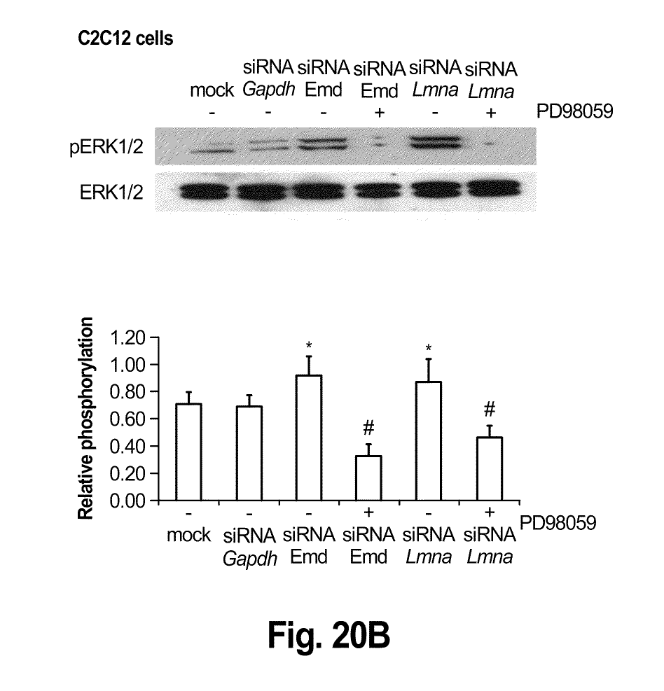

We analyzed the effect of a MEK inhibitor on ERK1/2 activity in HeLa and C2C12 cells treated with siRNA against Emd and Lmna. Cells were cultured with or without the addition of the MEK inhibitor PD98059 at a concentration of 45 μM for 24 hours. Immunoblotting with anti-pERK1/2 antibody demonstrated that the increase in pERK in HeLa and C2C12 cells treated with Emd and Lmna siRNAs was reduced when PD98059 is added to the culture medium (FIGS. 20A and 20B) Inhibition of ERK1/2 by PD98059 in siRNA-treated cells was also confirmed using an ELISA (FIG. 20B).

### Example 5

### Genetic Downregulation of MAPK Signaling

To show that genetic reductions of ERK improve the cardiac phenotype in Lmna^{H222P/H222P} mice, we cross Lmna^{H222P/H222P} mice to Erk1^{-/-} and Erk2^{+/-} mice (144-147). The progeny are Lmna^{H222P/H222P} mice that are completely deficient in ERK1 and have reduced levels of ERK2. PCR of DNA extracted from tail clippings is performed to determine genotypes of offspring. RT-PCR of RNA extracts and immunoblotting of protein extracts from cardiac muscle, skeletal muscle, and other tissues, confirms deficiency or reduced levels of ERK1 and ERK2. Approximately 16 each of male Lmna^{H222P/H222P}/Erk1^{+/+}Erk2^{+/+}, Lmna^{H222P/H222P}/Erk1^{-/-}/Erk2^{+/+}, and Lmna^{H222P/H222P}/Erk1^{+/+}/Erk2+/− mice are assessed by echocardiography and electrocardiography at 16 and 24 weeks of age.

We have shown that left ventricular tissue from Lmna H222P mice have a “molecular signature” of cardiomyopathy at the mRNA and protein expression level (96, 148; Example 1). These alterations in mRNA and protein expression occur prior to the onset of histological or clinical abnormalities in Lmna^{H222P/H222P} mice. We perform molecular analysis at the level of mRNA profiling and protein expression to examine the effects of ERKdeficiencies on a “molecular signature” indicative of cardiomyopathy in hearts of Lmna^{H222P/H222P} mice. Approximately 8 mice per group are sacrified at 16 or 25 weeks of age. RNA and proteins are isolated as described previously (96, 148, 149). Real-time RT-PCR is used to quantify mRNAs encoded by downstream genes in MAPK cascade. Proteins encoded by several of these RNAs are examined by immunoblotting. Expression of muscle-specific genes, such as those encoding myosins and sarcolipin, and fibrosis and inflammatory markers are also measured. We also measure the amounts of phosphorylated (active) and non-phosphorylated ERK using specific antibodies. Genetic reduction of ERK isoforms reduce or abolish the “molecular signature” indicative of cardiomyopathy and prevent dilated cardiomyopathy with heart block and skeletal muscle myopathy. These experiments are repeated with mice that are deficient in JNK1 and/or JNK2.

### REFERENCE FOR EXAMPLES 1-5 AND THE DETAILED DESCRIPTION OF THE INVENTION

The following references are referred to in this disclosure.
1. Emery, A. E. H. 2000. Emery-Dreifuss muscular dystrophy—a 40 year retrospective. Neuromusc. Disord. 10:228-232.
2. Bione, S., Maestrini, E., Rivella, S., Manchini, M., Regis, S., Romei, G., and Toniolo, D. 1994. Identification of a novel X-linked gene responsible for Emery-Dreifuss muscular dystrophy. Nature Genet. 8:323-327.
3. Manilal, S., Nguyen, T. M., Sewry, C. A., and Morris, G. E. 1996. The Emery-Dreifuss muscular dystrophy protein, emerin, is a nuclear membrane protein. Hum. Mol. Genet. 5:801-808.
4. Nagano, A., Koga, R., Ogawa, M., Kurano, Y., Kawada, J., Okada, R., Hayashi, Y. K., Tsukahara, T., and Arahata, K. 1996. Emerin deficiency at the nuclear membrane in patients with Emery-Dreifuss muscular dystrophy. Nature Genet. 12:254-259.
5. Bonne, G., Di Barletta, M. R., Varnous, S., Becane, H., Hammouda, E. H., Merlini, L., Muntoni, F., Greenberg, C. R., Gary, F., Urtizberea, J. A., et al. 1999. Mutations in the gene encoding lamin A/C cause autosomal dominant Emery-Dreifuss muscular dystrophy. Nature Genet. 21 :285-288.
6. Di Barletta, M. R. Ricci, E., Galluzzi, G., Tonali, P., Mora, M., Morandi, L., Romorini, A., Voit, T., Orstavik, K. H., Merlini, L., et al. 2000. Different mutations in the LMNA gene cause autosomal dominant and autosomal recessive Emery-Dreifuss muscular dystrophy. Am. J. Hum. Genet. 66:1407-1412.
7. Muchir, A., and Worman, H. J. 2004. The nuclear envelope and human disease. Physiology 19: 309-314.
8. Lin, F., and Worman, H. J. 1993. Structural organization of the human gene encoding nuclear lamin A and nuclear lamin C J. Biol. Chem. 268:16321-16326.
9. Aebi, U., Cohn, J., Buhle, L., and Gerace, L. 1986. The nuclear lamina is a meshwork of intermediate-type filaments. Nature 323:560-564.
10. Fisher, D. Z., Chaudhary, N., and Blobel, G. 1986. cDNA sequencing of nuclear lamins A and C reveals primary and secondary structural homology to intermediate filament proteins. Proc. Natl. Acad. Sci. USA. 83:6450-6454.
11. Goldman, A. E., Maul, G., Steinert, P. M., Yang, H. Y., and Goldman, R. D. 1986. Keratin-like proteins that co-isolate with intermediate filaments of BHK- 21 cells are nuclear lamins. Proc. Natl. Acad. Sci. USA. 83:3839-3843.
12. McKeon, F. D., Kirschner, M. W., and Caput, D. 1986. Homologies in both primary and secondary structure between nuclear envelope and intermediate filament proteins. Nature 319:463-468.
13. Lammerding, J., Schulze, P. C., Takahashi, T., Kozlov, S., Sullivan, T., Kamm, R. D., Stewart, C. L., and Lee, R. T. 2004. Lamin A/C deficiency causes defective nuclear mechanics and mechanotransduction. J. Clin. Invest. 113 :370-378.
14. Arimura, T., Helbling-Leclerc, A., Massart, C., Varnous, S., Niel, F., Lacene, E., Fromes, Y., Toussaint, M., Mura, A. M., Keller, D. I., et al. 2005. Mouse model carrying H222P-lmna mutation develops muscular dystrophy and dilated cardiomyopathy similar to human striated muscle laminopathies. Hum. Mol. Genet. 14:155-169.
15. Hwang, J. J., Allen, P. D., Tseng, G. C., Lam, C. W., Fananapazir, L., Dzau, V. J., and Liew, C. C. 2002. Microarray gene expression profiles in dilated and hypertrophic cardiomyopathic end-stage heart failure. Physiol. Genomics 10:31-44.
16. Mukherjee, S., Belbin, T. J., Spray, D. C., Iacobas, D. A., Weiss, L. M., Kitsis, R. N., Wittner, M., Jelicks, L. A., Scherer, P. E., Ding, A., et al. 2003. Microarray analysis of changes in gene expression in a murine model of chronic chagasic cardiomyopathy. Parasitol. Res. 91:187-196.
17. Barrans, J. D., Allen, P. D., Stamatiou, D., Dzau, V. J., and Liew, C. C. 2002. Global gene expression profiling of end-stage dilated cardiomyopathy using a human cardiovascular-based cDNA microarray. Am. J. Pathol. 160:2035-2043.
18. Grzeskowiak, R., Witt, H., Drungowski, M., Thermann, R., Hennig, S., Perrot, A., Osterziel, K. J., Klingbiel, D., Scheid, S., Spang, R., et al. 2003. Expression profiling of human idiopathic dilated cardiomyopathy. Cardiovasc. Res. 59:400-411.
19. Petrich, B. G., Gong, X., Lerner, D. L., Wang, X., Brown, J. H., Saffitz, J. E., and Wang, Y. 2002. c-Jun N-terminal kinase activation mediates downregulation of connexin43 in cardiomyocytes. Circ. Res. 91:640-647.
20. Petrich, B. G., Molkentin, J. D., and Wang, Y. 2003. Temporal activation of c-Jun N-terminal kinase in adult transgenic heart via cre-loxP-mediated DNA recombination. Faseb J. 17:749-751.
21. Chen, Z., Gibson, T. B., Robinson, F., Silvestro, L., Pearson, G., Xu, B., Wright, A., Vanderbilt, C., and Cobb, M. H. 2001. MAP kinases. Chem. Rev. 101:2449-2476.
22. Garrington, T. P., and Johnson, G. L. 1999. Organization and regulation of mitogen-activated protein kinase signaling pathways. Curr. Opin. Cell Biol. 11:211-218.
23. Baines, C. P., and Molkentin, J. D. 2005. STRESS signaling pathways that modulate cardiac myocyte apoptosis. J. Mol. Cell. Cardiol. 38:47-62.
24. Brunet, A., Roux, D., Lenormand, P., Dowd, S., Keyse, S., and Pouyssegur, J. 1999. Nuclear translocation of p42/p44 mitogen-activated protein kinase is required for growth factor-induced gene expression and cell cycle entry. Embo J. 18:664-674.
25. Hochholdinger, F., Baier, G., Nogalo, A., Bauer, B., Grunicke, H. H., and Uberall, F. 1999. Novel membrane-targeted ERK1 and ERK2 chimeras which act as dominant negative, isotype-specific mitogen-activated protein kinase inhibitors of Ras-Raf-mediated transcriptional activation of c-fos in NIH 3T3 cells. Mol. Cell. Biol. 19:8052-8065.
26. Qin, L. X., Beyer, R. P., Hudson, F. N., Linford, N. J., Morris, D. E., and Kerr, K. F. 2006. Evaluation of methods for oligonucleoyides array data via quantitative real time PCR. BMC Bioinformatics 7:23.
27. Millenaar, F. F., Okyere, J., May, S. T., van Zanten, M., Voesenek, L. A., and Peeters, A. J. 2006. How to decide? Different methods of calculating gene expression from short oligonucleotide array data will give different results. BMC Bioinformatics 7:137.
28. Widmann, C., Gibson, S., Jarpe, M. B., and Johnson, G. L. 1999. Mitogen-activated protein kinase: conservation of a three-kinase module from yeast to human. Physiol. Rev. 79:143-180.
29. Chi, H., Barry, S. P., Roth, R. J., Wu, J. J., Jones, E. A., Bennett, A. M., and Flavell, R. A. 2006. Dynamic regulation of pro- and anti-inflammatory cytokines by MAPK phosphatase 1 (MKP-1) in innate immune responses. Proc. Natl. Acad. Sci. USA 103:2274-2279.
30. Gillespie-Brown, J., Fuller, S. J., Bogoyevitch, M. A., Cowley, S., and Sugden, P. H. 1995. The mitogen-activated protein kinase kinase MEK1 stimulates a pattern of gene expression typical of the hypertrophic phenotype in rat ventricular cardiomyocytes. J. Biol. Chem. 270:28092-28096.
31. Thorburn, J., Carlson, M., Mansour, S. J., Chien, K. R., N G Ahn, N.G., and Thorburn, A. 1995. Inhibition of a signaling pathway in cardiac muscle cells by active mitogen-activated protein kinase kinase. Mol. Biol. Cell. 6:1479-1490.
32. Braz, J. C., Bueno, O. F., Liang, Q., Wilkins, B. J., Dai, Y. S., Parsons, S., Braunwart, J., Glascock, B. J., Klevitsky, R., Kimball, T. F., et al. 2003. Targeted inhibition of p38 MAPK promotes hypertrophic cardiomyopathy through upregulation of calcineurin-NFAT signaling. J. Clin. Invest. 111:1475-1486.
33. Nicol, R. L., Frey, N., Pearson, G., Cobb, M., Richardson, J., and Olson, E. N. 2001. Activated MEKS induces serial assembly of sarcomeres and eccentric cardiac hypertrophy. Embo J. 20:2757-2767.
34. Cook, S. A., Sugden, P. H., and Clerk, A. 1999. Activation of c-Jun N-terminal kinases and p38-mitogen-activated protein kinases in human heart failure secondary to ischaemic heart disease. J. Mol. Cell. Cardiol. 31:1429-1434.
35. Haq, S., Choukroun, G., Lim, H., Tymitz, K. M., del Monte, F., Gwathmey, J., Grazette, L., Michael, A., Hajjar, R., Force, T., et al. 2001. Differential activation of signal transduction pathways in human hearts with hypertrophy versus advanced heart failure. Circulation 103:670-677.
36. Rodriguez-Viciana, P., Tetsu, O., Tidyman, W. E., Estep, A. L., Conger, B. A., Santa Cruz, M., McCormick, F., and Rauen, K. A. 2006. Germline mutations in genes within the MAPK pathway cause cardio-facio-cutaneous syndrome. Science 311:1287-1290.
37. Bueno, O. F., De Windt, L. J., Tymitz, K. M., Witt, S. A., Kimball, T. R., Klevitsky, R., Hewett, T. E., Jones, S. P., Lefer, D. J., Peng, C. F., et al. 2000. The MEK1-ERK1/2 signaling pathway promotes compensated cardiac hypertrophy in transgenic mice. Embo J. 19:6341-6350.
38. Woodman, S. E., Park, D. S., Cohen, A. W., Cheung, M. W., Chandra, M., Shirani, J., Tang, B., Jelicks, L. A., Kitsis, R. N., Christ, G. J., et al. 2002. Caveolin-3 knock-out mice develop a progressive cardiomyopathy and show hyperactivation of the p42/44 MAPK cascade. J. Biol. Chem. 277:38988-38997.
39. Cohen, A. W., Park, D. S., Woodman, S. E., Williams, T. M., Chandra, M., Shirani, J., Pereira de Souza, A., Kitsis, R. N., Russell, R. G., Weiss, L. M., et al. 2003. Caveolin-1 null mice develop cardiac hypertrophy with hyperactivation of p42/44 MAP kinase in cardiac fibroblasts. Am. J. Physiol. Cell. Physiol. 284:457-474.
40. Luo, J., McMullen, J. R., Sobkiw, C. L., Zhang, L., Dorfman, A. L., Sherwood, M. C., Logsdon, M. N., Homer, J. W., DePinho, R. A., Izumo, S., et al. 2005. Class IA phosphoinositide 3-kinase regulates heart size and physiological cardiac hypertrophy. Mol. Cell. Biol. 25:9491-9502.
41. Malarkey, K., Belham, C. M., Paul, A., Graham, A., McLees, A., Scott, P. H., and Plevin, R. 1995. The regulation of tyrosine kinase signalling pathways by growth factor and G-protein-coupled receptors. Biochem J. 309:361-375.
42. Rosette, C., and Karin, M. 1996. Ultraviolet light and osmotic stress: activation of the JNK cascade through multiple growth factor and cytokine receptors. Science 274:1194-1197.
43. Coleman, M. L., Densham, R. M., Croft, D. R., and Olson, M. F. 2006. Stability of p21 (Waf1/Cip1) CDK inhibitor protein is responsive to RhoA-mediated regulation of the actin cytoskeleton. Oncogene 25: 2708-2716.
44. Smith, E. R., Smedberg, J. L., Rula, M. E., and Xu, X. X. 2004. Regulation of Ras-MAPK pathway mitogenic activity by restricting nuclear entry of activated MAPK in endoderm differentiation of embryonic carcinoma and stem cells. J. Cell Biol. 164:689-699.
45. Ivorra, C., Kubicek, M., González, J. M., Sanz-González, S. M., Álvarez-Barrientos, A., O'Connor, J. E., Burke, B., and Andres, V. 2006. A mechanism of AP-1 suppression through interaction of c-Fos with lamin A/C. Genes Dev. 20: 307-320.
46. Schwartz, K., Boheler, K. R., de la Bastie, D., Lompre, A. M., and Mercadier, J. J. 1992. Switches in cardiac muscle gene expression as a result of pressure and volume overload. Am. J. Physiol. 262:364-369.
47. Chien, K. R., Zhu, H., Knowlton, K. U., Miller-Hance, W., van-Bilsen, M., O'Brien, T. X., and Evans, S. M. 1993. Transcriptional regulation during cardiac growth and development. Annu. Rev. Physiol. 55 :77-95.
48. Hwang, J. J., Allen, P. D., Tseng, G. C., Lam, C. W., Fananapazir, L., Dzau, V. J., and Liew, C. C. 2002. Microarray gene expression profiles in dilated and hypertrophic cardiomyopathic end-stage heart failure. Physiol. Genomics 10:31-44.
49. Yung, C. K., Halperin, V. L., Tomaselli, G. F., and Winslow, R. L. 2004. Gene expression profiles in end-stage human idiopathic dilated cardiomyopathy: altered expression of apoptotic and cytoskeletal genes. Genomics 83:281-297.
50. Swynghedauw, B. 1999. Molecular mechanisms of myocardial remodeling. Physiol. Rev. 79:215-262.
51. Swynghedauw, B., and Baillard, C. 2000. Biology of hypertensive cardiopathy. Curr. Opin. Cardiol. 15:247-253.
52. Yoshimine, K., Horiuchi, M., Suzuki, S., Kobayashi, K., Abdul, J. M., Masuda, M., Tomomura, M., Ogawa, Y., Itoh, H., Nakao, K., et al. 1997. Altered expression of atrial natriuretic peptide and contractile protein genes in hypertrophied ventricle of JVS mice with systemic carnitine deficiency. J. Mol. Cell. Cardiol. 29:571-578.
53. Liao, P., Georgakopoulos, D., Kovacs, A., Zheng, M., Lerner, D., Pu, H., Saffitz, J., Chien, K., Xiao, R. P., Kass, D. A., and Wang, Y. 2001. The in vivo role of p38 MAP kinases in cardiac remodeling and restrictive cardiomyopathy. Proc. Natl. Acad. Sci. USA. 98:12283-12288.
54. Zheng, M., Dilly, K., Dos Santos Cruz, J., Li, M., Gu, Y., Ursitti, J. A., Chen, J., Ross, J. Jr., Chien, K. R., Lederer, J. W., and Wang, Y. 2004. Sarcoplasmic reticulum calcium defect in Ras-induced hypertrophic cardiomyopathy heart. Am. J. Physiol. Heart Circ. Physiol. 286:424-433.
55. Wang, J., Xu, N., Feng, X., Hou, N., Zhang, J., Cheng, X., Chen, Y., Zhang, Y., and Yang, X. 2005. Targeted disruption of Smad4 in cardiomyocytes results in cardiac hypertrophy and heart failure. Circ. Res. 97:821-828.
56. Takahashi, T., Allen, P. D., and Izumo, S. 1992. Expression of A-, B-, and C-type natriuretic peptide genes in failing and developing human ventricles. Correlation with expression of the Ca(2+)-ATPase gene. Circ. Res. 71:9-17.
57. Schwartz, K., de la Bastie, D., Bouveret, P., Oliviero, P., Alonso, S.,and Buckingham, M. 1986. Alpha-skeletal muscle actin mRNA's accumulate in hypertrophied adult rat hearts. Circ. Res. 59:551-555.
58. Izumo, S., Nadal-Ginard, B., Mandavi, V. 1988. Protooncogene induction and reprogramming of cardiac gene expression produced by pressure overload. Proc. Natl. Acad. Sci. USA. 85:339-343.
59. Ferrandi, C., Ballerio, R., Gaillard, P., Giachetti, C., Carboni, S., Vitte, P. A., Gotteland, J. P., and Cirillo, R. 2004 Inhibition of c-Jun N-terminal kinase decreases cardiomyocyte apoptosis and infarct size after myocardial ischemia and reperfusion in anaesthetized rats. Br. J. Pharmacol. 142 :953-960.
60. Borsello, T., Clarke, P. G., Hirt, L., Vercelli, A., Repici, M., Schorderet, D. F., Bogousslaysky, J., and Bonny, C. 2003. A peptide inhibitor of c-Jun N-terminal kinase protects against excitotoxicity and cerebral ischemia. Nature Med. 9:1180-1186.
61. Wang, J., Van De Water, T. R., Bonny, C., de Ribaupierre, F., Puel, J. L., and Zine, A. 2003. A peptide inhibitor of c-Jun N-terminal kinase protects against both aminoglycoside and acoustic trauma-induced auditory hair cell death and hearing loss. J. Neurosci. 23:8596-8607.
62. Minogue, A. M., Schmid, A. W., Fogarty, M. P., Moore, A. C., Campbell, V. A., Herron, C. E., and Lynch, M. A. 2003. Activation of the c-Jun N-terminal kinase signaling cascade mediates the effect of amyloid-beta on long term potentiation and cell death in hippocampus: a role for interleukin-lbeta? J. Biol. Chem. 278:27971-27980.
63. Kuida, K., and Boucher, D. M. 2004. Functions of MAP kinases: Insights from gene-targeting studies. J. Biochem. 135: 653-656.
64. Mounkes, L. C., Kozlov, S. V., Rottman, J. N., and Stewart, C. L. 2005. Expression of an LMNA-N195K variant of A-type lamins results in cardiac conduction defects and death in mice. Hum. Mol. Genet. 14:2167-2180.
65. Pavlidis, P., Lewis, D. P., and Noble, W. S. 2002. Exploring gene expression data with class scores. Pac. Symp. Biocomput. 474-485.
66. Dennis, G. Jr., Sherman, B. T., Hosack, D. A., Yang, J., Gao, W., Lane, H. C., and Lempicki, R. A. 2003. DAVID: Database for Annotation, Visualization, and Discovery. Genome Biology. 4:P3.
67. Ponchel, F., Toomes, C., Bransfield, K., Leong, F. T., Douglas, S. H., Field, S. L., Bell, S. M., Combaret, V., Puisieux, A., Mighell, A. J., et al. 2003. Real-time PCR based on SYBR-Green I fluorescence: An alternative to the TaqMan assay for a replicative quantification of gene rearrangements, gene amplifications and micro gene deletions. BMC Biotechnol. 13:18.
68. Fatkin, D., MacRae, C, Sasaki, T., Wolff, M. R., Porcu, M., Frenneaux, M., Atherton, J., Vidaillet, H. J., Spudich, S., De Girolami, U., et al. 1999. Missense mutations in the rod domain of the lamin A/C gene as causes of dilated cardiomyopathy and conduction-system disease. N. Engl. J. Med. 341:1715-1724.
69. Muchir, A., Bonne, G., van der Kooi, A. J., van Meegen, M., Baas, F., Bolhuis, P. A., de Visser, M., and Schwartz, K. 2000. Identification of mutations in the gene encoding lamins A/C in autosomal dominant limb girdle muscular dystrophy with atrioventricular conduction disturbances (LGMD1B). Hum. Mol. Genet. 9:1453-1459.
70. De Sandre-Giovannoli, A., Chaouch, M., Kozlov, S., Vallat, J. M., Tazir, M., Kassouri, N., Szepetowski, P., Hammadouche, T., Vandenberghe, A., Stewart, C. L., et al. 2002. Homozygous defects in LMNA, encoding lamin A/C nuclear envelope proteins, cause autosomal recessive axonal neuropathy in human (Charcot-Marie-Tooth Disorder Type 2) and mouse. Am. J. Hum. Genet. 70:726-736.
71. Cao, H., and Hegele, R. A. 2000. Nuclear lamin A/C R482Q mutation in Canadian kindreds with Dunnigan-type familial partial lipodystrophy. Hum. Mol. Genet. 9:109-112.
72. Shackleton, S., Lloyd, D. J., Jackson, S. N., Evans, R., Niermeijer, M. F., Singh, B. M., Schmidt, H., Brabant, G., Kumar, S., Durrington, P. N., et al. 2000. LMNA, encoding lamin A/C, is mutated in partial lipodystrophy. Nature Genet. 24:153-156.
73. Speckman, R. A., Garg, A., Du, F., Bennett, L., Veile, R., Arioglu, E., Taylor, S. I., Lovett, M., and Bowcock, A. M. 2000. Mutationai and hapiotype analyses of families with familial partial lipodystrophy (Dunnigan variety) reveal recurrent missense mutations in the globular C-terminal domain of lamin A/C. Am. J. Hum. Genet. 66:1192-1198.
74. Dunnigan, M. G., Cochrane, M. A., Kelly, A., and Scott, J. W. 1974. Familial lipoatrophic diabetes with dominant transmission. A new syndrome. Q. J. Med. 43:33-48.
75. Novelli, G., Muchir, A., Sangiuolo, F., Helbiing-Leclerc, A., Rosaria d'Apice, M., Massart, C, Capon, F., Sbraccia, P., Federici, M., Lauro, R., et al. 2002. Mandibuloacral dysplasia is caused by a mutation in LMNA encoding lamins A/C. Am. J. Hum. Genet. 71:426-431.
76. Eriksson, M., Brown, W. T., Gordon, L. B., Glynn, M. W., Singer, J., Scott, L, Erdos, M. R., Robbins, C. M., Moses, T. Y., Berglund, P., et al. 2003. Recurrent de novo point mutations in lamin A cause Hutchinson-Gilford progeria syndrome. Nature 423 :293-298.
77. De Sandre-Giovannoli, A., Bernard, R., Cau, P., Navarro, C, Amiel, J., Boccaccio, I., Lyonnet, S., Stewart, C. L., Munnich, A., Le Merrer, M., et al. 2003. Lamin A truncation in Hutchinson-Gilford progeria. Science 300:2055.
78. Chen, L., Lee, L., Kudlow, B., Dos Santos, H., Sletvold, O., Shafeghati, Y., Botha, E., Garg, A., Hanson, N., Martin, G., et al. 2003. LMNA mutations in atypical Werner's syndrome. Lancet 362:440-445.
79. Navarro, C. L., De Sandre-Giovannoli, A., Bernard, R., Boccaccio, I., Boyer, A., Genevieve, D., Hadj-Rabia, S., Gaudy-Marqueste, C, Smitt, H. S., Vabres, P., et al. 2004. Lamin A and ZMPSTE24 (FACE-1) defects cause nuclear disorganization and identify restrictive dermopathy as a lethal neonatal laminopathy. Hum. Mol. Genet. 13:2493-2503.
80. Bennett, B. L., Sasaki, D. T., Murray, B. W., O'Leary, E. C., Sakata, S T., Xu, W., Leisten, J. C., Motiwala, A., Pierce, S., Satoh, Y., et. al. 2001. SP600125, an anthrapyrazolone inhibitor of Jun N-terminal kinase. Proc. Natl. Acad. Sci. USA. 98:13681-13686.
81. Han, Z., Boyle, D. L., Chang, L., Bennett, B., Karin, M., Yang, L., Manning, A. M., and Firestein, G. S. 2001. c-Jun N-terminal kinase is required for metalloproteinase expression and joint destruction in inflammatory arthritis. J. Clin. Invest. 108:73-81.
82. Shin, M., Yan, C, and Boyd D. 2002. An inhibitor of c-jun aminoterminal kinase (SP600125) represses c-Jun activation, DNA-binding and PMA-inducible 92-kDa type IV collagenase expression. Biochim, Biophys. Acta. 1589:311-316.
83. Dudley, D. T., Pang, L, Decker, S. J., Bridges, A. J., and Saltiel, A. R. 1995. A synthetic inhibitor of the mitogen-activated protein kinase cascade. Proc. Natl. Acad. Sci. USA. 92:7686-7689.
84. Pang, L., Sawada, T., Decker, S. J., and Saltiel, A. R. 1995 Inhibition of MAP kinase kinase blocks the differentiation of PC-12 cells induced by nerve growth factor. J. Biol. Chem. 270:13585-13588.
85. Waters, S. B., Holt, K. H., Ross, S. E., Syu, L. J., Guan, K. L., Saltiel, A. R., Koretzky, G. A., and Pessin, J. E. 1995. Desensitization of Ras activation by a feedback disassociation of the SOS-Grb2 complex. J. Biol. Chem. 270:20883-20886.
86. Langlois, W. J., Sasaoka, T., Saltiel, A. R., and Olefsky, J. M. 1995. Negative feedback regulation and desensitization of insulin- and epidermal growth factor-stimulated p21ras activation. J. Biol. Chem. 270:25320-25323.
87. Kultz, D., Madhany, S., and Burg, M. B. 1998. Hyperosmolality causes growth arrest of murine kidney cells. Induction of GADD45 and GADD 153 by osmosensing via stress-activated protein kinase 2. J. Biol. Chem. 273:13645-13651.
88. Means, T. K., Pavlovich, R. P., Roca, D., Vermeulen, M. W., and Fenton, M J. 2000. Activation of TNF-alpha transcription utilizes distinct MAP kinase pathways in different macrophage populations. J. Leukoc. Biol. 67:885-893.
89. Ostlund, C, Bonne, G., Schwartz, K., and Worman, H. J. 2001. Properties of lamin A mutants found in Emery-Dreifuss muscular dystrophy, cardiomyopathy and Dunnigan-type partial lipodystrophy. J. Cell Sci. 114:4435-4445.
90. Boguslaysky, R. L., Stewart, C. L., Worman, H. J. 2006. Nuclear lamin A inhibits adipocyte differentiation: implications for Dunnigan-type familial partial lipodystrophy. Hum. Mol. Genet. 15:653-663.
91. Paradisi, M., McClintock, D., Boguslaysky, R. L., Pedicelli, C, Worman, H. J., and Djabaii, K. 2006. Dermal fibroblasts in Hutchinson-Gilford progeria syndrome with the lamin A G608G mutation have dysmorphic nuclei and are hypersensitive to heat stress. BMC Cell Biol. 6:27.
92. Laemmli, U. K. 1970. Cleavage of structural proteins during the assembly of the head of bacteriophage T4. Nature 227:680-685.
93. Daigle, C, Martens, F. M., Girardot, D., Dao, H. H., Touyz, R. M., and Moreau, P. 2004. Signaling of angiotensin 11-induced vascular protein synthesis in conduit and resistance arteries in vivo. BMC Cardiovasc Disord. 10; 4:6.
94. Wang, Y., Herron, A. J., and Worman, H. J. 2006. Pathology and nuclear abnormalities in hearts of transgenic mice expressing M371K lamin A encoded by an LMNA mutation causing Emery-Dreifuss muscular dystrophy. Hum. Mol. Genet. 15(16):2479-89.
95. Kaplan, E. L., and Meier, P. 1958. Nonpararnetric estimation from incomplete observations. J. Amer. Statist. Assn. 53:457-481.
96. Muchir, A. et al., Activation of MAPK pathways links LMNA mutations to cardiomyopathy in Emery-Dreifuss muscular dystrophy. J. Clin. Invest. 117, 1282 (2007).
97. Kyoi, S., Otani, H., Matsuhisa, S., Akita, Y., Tatsumi, K., Enoki, C., et al. (2006). Opposing effects of p38 MAP kinase and JNK inhibitors on the development of heart failure in the cardiomyopathic hamster. Cardiovasc Res 69, 888-898.
98. Lorusso, P. M. et al., J. Clin. Oncol. 23, 5281 (2005).
99. Adjei, A. A. et al., J. Clin. Oncol. 26, 2139 (2008).
100. Miller R G., Layzer R B, Mellenthin M A, Golabi M, Francoz R A, Mall J C: Emery-Dreifuss muscular dystrophy with autosomal dominant transmission. Neurology 1985, 35:1230-1233.
101. Sullivan T, Escalante-Alcalde D, Bhatt H, Anver M, Bhat N, Nagashima K, Stewart C L, Burke B: Loss of A-type lamin expression compromises nuclear envelope integrity leading to muscular dystrophy. J Cell Biol 1999, 147:913-920.
102. de Nadal E, Alepuz P M, Posas F: Dealing with osmostress through MAP kinase activation. EMBO Rep 2002, 3:735-40
103. Cowan K J, Storey K B: Mitogen-activated protein kinases: new signaling pathways functioning in cellular responses to environmental stress. J Exp Biol 2003, 206:1107-15.
104. Lammerding J, Hsiao J, Schulze P C, Kozlov S, Stewart C L, Lee R T: Abnormal nuclear shape and impaired mechanotransduction in emerin-deficient cells. J Cell Biol 2005, 170:781-791.
105. Muchir A, van Engelen B G M, Lammens M, Mislow J M, McNally E, Schwartz K, Bonne G: Nuclear envelope alterations in fibroblasts from LGMD1B patients carrying nonsense Y259X heterozygous or homozygous mutation in lamin A/C gene. Exp Cell Res 2003, 291:352-362.
106. Ozawa R, Hayashi Y K, Ogawa M, Kurokawa R, Matsumoto H, Noguchi S, Nonaka I, Nishino I: Emerin-lacking mice show minimal motor and cardiac dysfunctions with nuclear-associated vacuoles. Am J Pathol 2006, 168:907-917.
107. Melcon G, Kozlov S, Cutler D A, Sullivan T, Hernandez L, Zhao P, Mitchell S, Nader G, Bakay M, Rottman J N, Hoffman E P, Stewart C L: Loss of emerin at the nuclear envelope disrupts the Rb1/E2F and MyoD pathways during muscle regeneration. Hum Mol Genet 2006, 15:637-651.
108. Nikolova V, Leimena C, McMahon A C, Tan J C, Chandar S, Jogia D, Kesteven S H, Michalicek J, Otway R, Verheyen F, Rainer S, Stewart C L, Martin D, Feneley M P, Fatkin D. Defects in nuclear structure and function promote dilated cardiomyopathy in lamin A/C-deficient mice. J Clin Invest 2004, 113:357-369.
109. Ishitani T, Ninomiya-Tsuji J, Nagai S, Nishita M, Meneghini M, Barker N, Waterman M, Bowerman B, Clevers H, Shibuya H, Matsumoto K: The TAK1-NLK-MAPK-related pathway antagonizes signalling between beta-catenin and transcription factor TCF. Nature 1999, 399:798-802.
110. Kim D, Rath O, Kolch W, Cho K H: A hidden oncogenic positive feedback loop caused by crosstalk between Wnt and ERK Pathways. Oncogene 2007, 26:4571-4579.
111. Massague J: How cells red Tgf-β signals. Nat Rev Mol Cell Biol 2000, 1:169-78.
112. Hocevar B A, Brown T L, Howe PH: Tgf-β induced fibronectin synthesis through a c-Jun N-terminal kinase-dependent, SMAD4-independent pathway. The EMBO J 1999, 18:1345-1356.
113. Wolf C M, Wang L, Alcalai R, Pizard A, Burgon P G, Ahmad F, Sherwood M, Branco D M, Wakimoto H, Fishman G I, See V, Stewart C L, Conner D A, Berul C I, Seidman C E, Seidman J G: Lamin A/C haploinsufficiency causes dilated cardiomyopathy and apoptosis-triggered cardiac conduction system disease. J Mol Cell Cardiol 2008, 44:293-303.
114. Yoon S, Seger R: The extracellular signal-regulated kinase: Multiple substrates regulate diverse cellular functions. Growth Factors 2006, 24:21-44.
115. Torres M, Forman H J: Redox signaling and the MAP kinase pathways. Biofactors 2003, 17:287-96
116. Michel M C, Li Y, Heusch G: Mitogen-activated protein kinases in the heart. Naunyn-Schmiedeberg Arch Pharmacol 2001, 363:245-266.
117. Crisp M, Liu Q, Roux K, Rattner J B, Shanadan C, Burke B, Stahl P D, Hodzic D: Coupling of the nucleus and cytoplasm: role of the LILAC complex. J Cell Biol 2006, 172:41-53.
118. English J M, Cobb M H: Pharmacological inhibitors of MAPK pathways. Trends Pharmacol Sci 2002, 23:40-45.
119. U.S. Pat. No. 6,924,415 “Transgenic mice comprising a constitutively-activated MEK5 and exhibiting cardiac hypertrophy and dilated cardiomyopathy”
120. Barancik, M., Htun, P., Srrohm, C., Kilian, S., & Schaper, W. (2000) Inhibition of the cardiac g38-MAPK pathway by SB203580 delays ischemic cell death. J Cardiovasc Pharmacol 35, 474-483.
121. Sanada, S., Kitakaze, M., Papst, P. J., Hatanaka, K., Asanuma, H., Aki, T., et al. (2001). Role of Phasic Dynamism of p38 Mitogen-Activated Protein Kinase Activation in Ischemic Preconditioning of the Canine Heart. Circ Res 88, 175-180.
122. See, F., Thomas, W.,Way, K., Tzanidis, A., Kompa, A., Lewis, D., et al. (2004). p38 mitogen-activated protein kinase inhibition improves cardiac function and attenuates left ventricular remodeling following myocardial infarction in the rat. J Am Coll Cardiol 44, 1679-1689.
123. Yada, M., Shimamoto, A., Hampton, C. R., Chong, A. J., Takayama, H., Rothnie, C. L., et al. (2004). FR167653 diminishes infarct size in a murine model of myocardial ischemia-reperfusion injury. J Thorac Cardiovasc Surg 128, 588-594.
124. Liu, Y. H., Wang, D., Rhaleb, N. E., Yang, X. P., Xu, J., Sankey, S. S., et al. (2005). Inhibition of p38 mitogen-activated protein kinase protects the heart against cardiac remodeling in mice with heart failure resulting from myocardial infarction. J Card Fail 11, 74-81.
125. Engel, F. B., Hsieh, P. C., Lee, R. T., & Keating, M. T. (2006). FCF1/p38 MAP kinase inhibitor therapy induces cardiomyocyte mitosis, reduces scarring, and rescues function after myocardial infarction. Proc Natl Acad Sci USA 103, 15546-15551.
126. Li, M., Georgakopoulos, D., Lu, G., Hester, L., Kass, D. A, Hasday, J., et al. (2005). p38 MAP kinase mediates inflammatory cytokine induction in cardiomyocytes and extracellular matrix remodeling in heart. Circulation 111, 2494-2 502.
127. Nakamura, T., Colbert, M., Krenz, M., Molkentin, J. D., Hahn, H. S., Dorn, G. W. 2nd, Robbins, J. (2007) Mediating ERK 1/2 signaling rescues congenital heart defects in a mouse model of Noonan syndrome. J Clin Invest 117, 2123-2132.
128. Worman, H. J. and Bonne, G (2007) “Laminopathies”: a wide spectrum of human diseases. Exp. Cell Res., 313, 2121-2133.
129. Taylor, M. R., Fain, P. R., Sinagra, G., Robinson, M. L., Robertson, A. D., Carniel, E., Di Lenarda, A., Bohlmeyer, T. J., Ferguson, D. A., Brodsky, G. L. et al. (2003) Natural history of dilated cardiomyopathy due to lamin A/C gene mutations. J. Am. Coll. Cardiol., 41, 771-780.
130. Becane, H. M., Bonne, G., Varnous, S., Muchir, A., Ortega, V., Hammouda, E. H., Urtizberea, J. A., Lavergne, T., Fardeau, M., Eymard, B. et al. (2000) High incidence of sudden death with conduction system and myocardial disease due to lamins A and C gene mutation. Pacing Clin. Electrophysiol., 23, 1661-1666.
131. Meune, C., Van Berlo, J. H., Anselme, F., Bonne, G., Pinto, Y. M. and Duboc D. (2006) Primary prevention of sudden death in patients with lamin A/C gene mutations. N. Engl. J. Med., 354, 209-210.
132. Sanada, S., Node, K., Minamino, T., Takashima, S., Ogai, A., Asanuma, H., Ogita, H., Liao, Y., Asakura, M., Kim, J. et al. (2003) Long-acting Ca²⁺ blockers prevent myocardial remodeling induced by chronic NO inhibition in rats. Hypertension, 41, 963-967.
133. Mizuno, Y., Yoshimura, M., Harada, E., Nakayama, M., Sakamoto, T., Shimasaki, Y., Ogawa, H., Kugiyama, K., Saito, Y., Nakao, K. et al. (2000) Plasma levels of A- and B-type natriuretic peptides in patients with hypertrophic cardiomyopathy or idiopathic dilated cardiomyopathy. Am. J. Cardiol., 86, 1036-1040.
134. Abraham, W. T., Gilbert, E. M., Lowes, B. D., Minobe, W. A., Larrabee, P., Roden, R. L., Dutcher, D., Sederberg, J., Lindenfeld, J. A., Wolfel, E. E. et al. (2002) Coordinate changes in myosin heavy chain isoform gene expression are selectively associated with alterations in dilated cardiomyopathy phenotype. Mol. Med., 8, 750-760.
135. Favreau, C., Delbarre, E., Courvalin, J. C. and Buendia B. (2008) Differentiation of C2C12 myoblasts expressing lamin A mutated at a site responsible for Emery-Dreifuss muscular dystrophy is improved by inhibition of the MEK-ERK pathway and stimulation of the PI3-kinase pathway. Exp. Cell Res., 314, 1392-1405.
136. Alessi, D. R., Cuenda, A., Cohen, P., Dudley, D. T. and Saltiel, A. R. (1995) PD 098059 is a specific inhibitor of the activation of mitogen-activated protein kinase kinase in vitro and in vivo. J. Biol. Chem., 270, 27489-27494.
137. Börsch-Haubold, A. G., Pasquet, S. and Watson, S. P. (1998) Direct inhibition of cyclooxygenase-1 and -2 by the kinase inhibitors SB 203580 and PD 98059. SB 203580 also inhibits thromboxane synthase. J. Biol. Chem., 273, 28766-28772.
138. Mamdani, M., Juurlink, D. N., Lee, D. S., Rochon, P. A., Kopp, A., Naglie, G., Austin, P. C., Laupacis, A. and Stukel, T. A. (2004) Cyclo-oxygenase-2 inhibitors versus non-selective non-steroidal anti-inflammatory drugs and congestive heart failure outcomes in elderly patients: a population-based cohort study. Lancet, 363, 1751-1756.
139. Hudson, M., Richard, H. and Pilote, L. (2005) Differences in outcomes of patients with congestive heart failure prescribed celecoxib, rofecoxib, or non-steroidal anti-inflammatory drugs: population based study. BMJ, 330, 1370.
140. Manilal, S., Nguyen, T. M., Sewry, C. A. and Morris, G. E. (1996) The Emery-Dreifuss muscular dystrophy protein, emerin, is a nuclear membrane protein. Hum. Mol. Genet., 5, 801-808.
141. Yang, S. H., Meta, M., Qiao, X., Frost, D., Bauch, J., Coffinier, C., Majumdar, S., Bergo, M. O., Young, S. G. and Fong, L. G. (2006) A farnesyltransferase inhibitor improves disease phenotypes in mice with a Hutchinson-Gilford progeria syndrome mutation. J. Clin. Invest., 116, 2115-2121.
142. Yang, S. H., Qiao, X., Fong, L. G. and Young, S. G. (2008) Treatment with a farnesyltransferase inhibitor improves survival in mice with a Hutchinson-Gilford progeria syndrome mutation. Biochim. Biophys. Acta, 1781, 36-39.
143. Lo Russo, P. M., Adjei, A. A., Varterasian, M., Gadgeel, S., Reid, J., Mitchell, D. Y., Hanson, L., DeLuca, P., Bruzek, L., Piens, J. et al. (2005) Phase I and pharmacodynamic study of the oral MEK inhibitor CI-1040 in patients with advanced malignancies. J. Clin. Oncol., 23, 5281-5293.
144. Pages, G. et al. (1999) Defective thymocyte maturation in p44 MAP kinase (Erk1) knockout mice. Science 286:1374-1377.
145. Yao, Y. et al. (2003) Extracellular signal-regulated kinase 2 is necessary for mesoderm differentiation. Proc. Natl. Acad. Sci. U.S.A. 100:12759-12764.
146. Hatano, N. et al. (2003) Essential role for ERK2 mitogen-activated protein kinase in placental development. Genes Cells 8:847-856.
147. Saba-El-Leil, M. K. et al. (2003) An essential function of the mitogen-activated protein kinase Erk2 in mouse trophoblast development. EMBO Rep. 4:964-968.
148. Muchir, A. et al. (2009) Inhibition of extracellular signal-regulated kinase signaling to prevent cardiomyopathy caused by mutation in the gene encoding A-type lamins Hum. Mol. Genet. 18:241-247.
149. Muchir, A. et al. (2007) Activation of MAPK in hearts of Emd null mice: similarities between mouse models of X-linked and autosomal dominant Emery-Dreifuss muscular dystrophy. Hum. Mol. Genet. 16: 1884-1895.
150. Heinke, J., Molkentin, J. D. (2006) Regulation of cardiac hypertrophy by intracellular signaling pathways. Nat. Rev. Mol. Cell. Biol. 7:589-600.
151. Maosong & Elion (2005) MAP kinase pathways. J. Cell. Sci. 118: 3569-3572.
152. Chang & Karin (2001) Mammalian MAP kinase signaling cascade. Nature 410: 37-40.
153. Chen et al. (2000) The c-Jun N-terminal kinase pathway and apoptotic signaling. Int. J. Oncol. 16: 651-62.
154. Pearson et al. (2001) Mitogen-activated protein (MAP) kinase pathways: regulation and physiological functions. Endocr. Rev. 22: 53-83.
155. Davis et al. (2000) Signal transduction by the JNK group of MAP kinases. Cell 103: 239-252.
156. Roux & Blenis (2004) ERK and p38 MAPK-activated protein kinases: a family of protein kinases with diverse biological functions. Microbiol. Mol. Biol. Rev. 68: 320-344.
157. Hetman, M., et al. (2002) J. Biol. Chem. 277, 49577.
158. Ohno, M., et al. (2001) Nat. Neurosci. 4, 1238.
159. Wang, H., et al. (2001) Biochem. Biophys. Res. Commun. 286, 869.
160. Scherle, P. A., et al. (2000) J. Biol. Chem. 275, 37086.
161. Valjent, E., et al. (2000) J. Neurosci. 20, 8701.
162. Watabe, A. M., et al. (2000) J. Neurosci. 20, 5924.
163. Atkins, C. M., et al. (1998) Nat. Neurosci. 1, 602.
164. DeSilva, D. R., et al. 1998. J. Immunol. 160, 4175.
165. Duncia, J. V., et al. 1998. Biorg. Med. Chem. Lett. 8, 2839.
166. Favata, M. F., et al. 1998. J. Biol. Chem. 273, 18623.
167. Ahn et al. (1999) Promega Notes. 71: 4.
168. Kohno & Pouyssegur (2003) Pharmacological inhibitirs of the ERK signaling pathway: application as anticancer drugs. Prog. Cell. Cyc. Res. 5: 219-224.
169. Ohori, M. et al. (2005) Identification of a selective ERK inhibitor and structural determination of the inhibitor-ERK2 complex. Biochem. Biophys. Res. Comm. 336: 357-363.
170. Chen, F. et al. (2006) Characterization of ATP-independent ERK inhibitors identified through in silico analysis of the active ERK2 structure. Bioorg. Med. Chem. 16:6281-6288.
171. Hancock, C. N. et al. (2005) Identification of novel extracellular signal-regulated kinase docking domain inhibitors. J. Med. Chem. 48: 4586-4595.
172. National Cancer Institute NCI Drug Dictionary, available at www.cancer.gov.
173. Stebbins, J. L. et al. (2008) Identification of a new JNK inhibitor targeting the JNK-JIP interaction site. Proc. Natl. Acad. Sci., U.S.A. 105: 16809-16813.
174. Wiegler, K. et al. (2008) The JNK inhibitor XG-102 protects from ischemic damage with delayed intravenous administration also in the presence of recombinant tissue plasminogen activator. Cerebrovasc. Dis. 26: 360-366.
175. Carboni, S. et al. (2004) AS601245: a JNK inhibitor with neuroprotective properties. J. Pharmacol. Exp. Ther. 310: 25-32.

### Example 6

### Second-Generation MEK1/2 Inhibitors Prevented LMNA-Associated Cardiomyopahy

The numbers in brackets below refer to the references in the numbered list that follows this Example 6.

### Chemical Entities

The study in Example 6 tested compound(s) with superior pharmacological properties that can be used as drug(s) for EDMD and CMD1A in humans. We focused on re-profiling MAP kinase inhibitors that are already in human clinical trials for other indications for treating cardiomyopathy in Lmna^{H222P/H222P} mice (Table 2). Although several biotechnology and pharmaceutical companies are working on bringing MAP kinase inhibitors to market [15,16], these are being developed mostly for cancer or inflammation and not for cardiomyopathy. MEK1/2 inhibitors share common and special features, which make them good compounds to develop into drugs [16].

The vast majority of kinase inhibitors bind to the ATP binding site, and this causes selectivity issues as this site is highly conserved in all kinases. This leads to multi-kinase inhibitors. In contrast, known MEK1/2 inhibitors are allosteric binders; they do not bind to the ATP binding site and do not have to compete with endogenous ATP. Therefore, MEK1/2 inhibitors can be highly selective. All of the candidate MAP kinase inhibitors used in this Example 6 were selected based on their pharmacokinetics and toxicity profiles.

CI-1040 (PD184352), a benzhydroxamate from Pfizer, was the first small-molecule MEK inhibitor that proceeded to clinical testing [17]. It was developed based on compounds and structures identified during the screening that led to the identification of PD98059, but had improved potency and selectivity. C1-1040 selectively inhibited MEK in a noncompetitive manner with respect to ATP, by binding to a pocket adjacent to the ATP binding site. C1-1040, shown below, is an oral MEK inhibitor with promising preclinical activity that led to its clinical development in patients with advanced solids tumors (including lymphoma) [18,19].

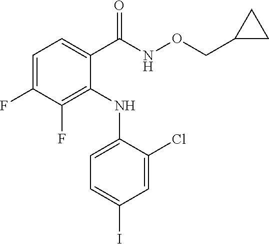

PD0325901, shown below, is a second-generation oral MEK inhibitor that was subsequently developed by Pfizer. Relatively minor changes distinguish the chemical structure of PD0325901 from that of CI-1040 [17]. Nevertheless, these minor structural changes imparted significant increases in potency with PD0325901. Pre-clinical findings of significantly improved pharmacologic and pharmaceutical properties of PD0325901 showed the compound as a therapeutic agent [20,21].

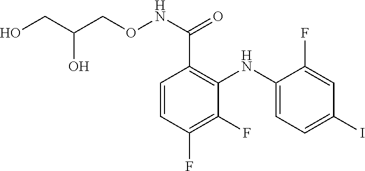

ARRY-142886 (AZD6244; Array Biopharma/AstraZeneca), shown below, is another potent, highly specific MEK inhibitor [22]. ARRY-142886 has undergone phase I testing in a trial of patients with solid tumor types [23]. Promising results in this phase I clinical trial triggered a phase II study, which is currently under investigation.

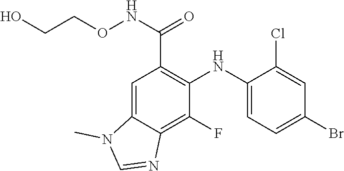

TABLE 10
Description of MEK1/2 inhibitors

| | PD0325901 | AZD6244 | CI-1040 |
|---|---|---|---|
| Company | Pfizer | Array BioPharma | Pfizer |
| | | (AstraZeneca) | |
| IC50 | 1 nM | 10 nM | 1 nM |
| Development | Phase I | Phase II | Phase II |
| | (discontinued) | (ongoing) | (discontinued) |
| | Non-Randomized, | Randomized, | Non-Randomized, |
| | Open Label | Open Label | Open Label |
| Clinical | NCT00147550 | NCT00514761 | NCT00033384 |
| Trial | NCT00174369 | | NCT00034827 |
| number | | | |
| Dose (daily) | 0.01 mg/kg- | 1 mg/kg- | 1 mg/kg- |
| | 0.5 mg/kg | 5 mg/kg | 20 mg/kg |
| Indication | Cancer | Cancer | Cancer |

### Methodology

To evaluate the effect of chronic treatment with the inhibitors, Lmna^{H222P/H222P} mice were randomly assigned to four treatment groups: vehicle (DMSO) (n=12), PD0325901 (n=11), AZD6244 (n=9) and CI-1040 (n=6 per molecule). The compounds were administered at a dose of 1 mg/kg/day, 5 days a week to Lmna^{H222P/H222P} mice, as previously described [12,13]. Treatment was initiated at 16 weeks of age and continued until the mice were 20 weeks of age. At 20 weeks of age, the mice were analyzed by echocardiography and then sacrificed for biochemical studies. Untreated male Lmna^{+/+} and Lmna^{H222P/H222P} mice were similarly analyzed for comparisons.

### Pharmacology

Before and after the 4 weeks treatment with PD0325901, AZD6244 and CI-1040 MEK1/2 inhibitors, we collected serum from male Lmna^{H222P/H222P} mice to measure the concentration of the chemical entities present in the serum. We were able to detect between 0.17 μM and 1.22 μM of AZD6244 in the Lmna^{H222P/H222P} mice serum at the end of the treatment. We are currently measuring the circulating concentration for PD0325901 and CI-1040.

### Results

### 1) Effect of the MEK1/2 Inhibitors on Their Molecular Target

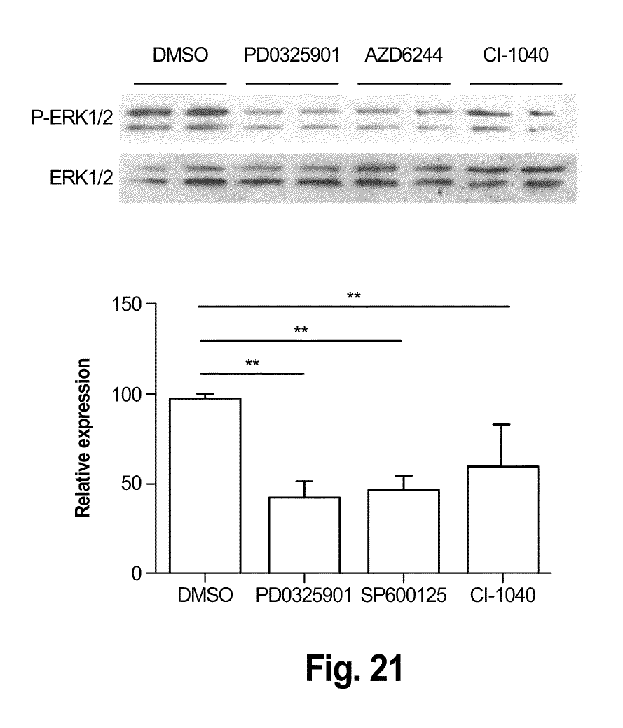

We assessed the efficacy of the chemical entities in inhibiting their targets in the heart by immunoblot using anti-total ERK1/2 (Santa Cruz) and anti-pERK1/2 (Cell Signaling) antibodies [12,13]. Systemic administration of PD0325901, AZD6244 and CI-1040 inhibited partially the phosphorylation of ERK1 and ERK2 in hearts, as shown by immunoblotting of proteins in tissue homogenates with antibodies against phosphorylated ERK1/2 and total ERK1/2 (FIG. 21).

### 2) Effect of the MEK1/2 Inhibitors on the Regulation of Cardiac Hormones

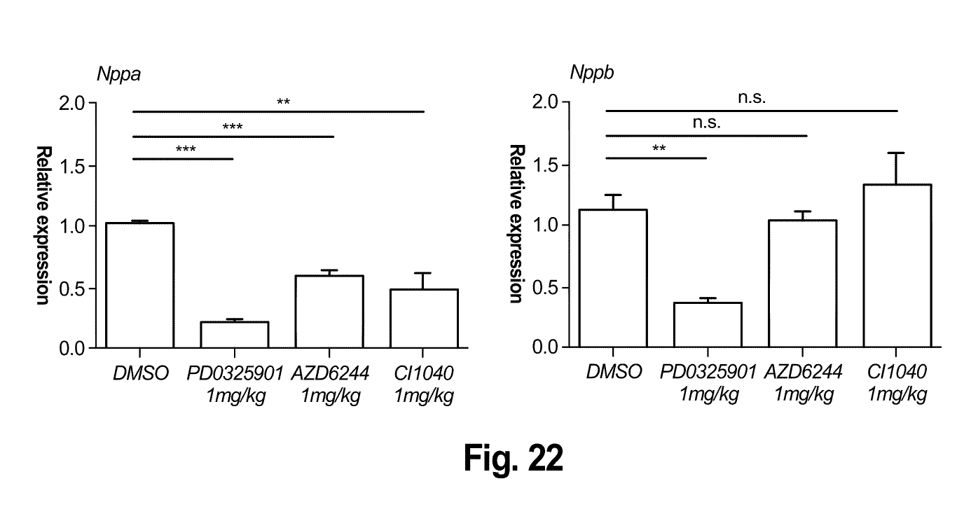

A feature of dilated cardiomyopathy is the up-regulation of cardiac hormones such as natriuretic peptides (atrial and brain natriuretic factors) as a compensatory mechanism to maintain cardiac output [24,25]. The Lmna^{H222P/H222P} mice treated with with the three MEK1/2 inhibitors had a cardiac expression of atrial natriuretic factor (Nppa) decreased compared to DMSO-treated Lmna^{H222P/H222P} mice (FIG. 22). Only PD0325901-treated Lmna^{H222P/H222P} mice had a cardiac expression of brain natriuretic factor (Nppb) decreased compared to Lmna^{+/+} mice (FIG. 22).

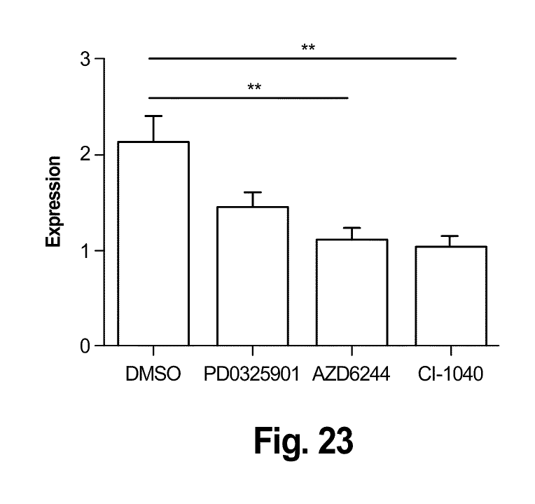

We measured the expression of atrial-natriuretic factor in the serum Lmna^{H222P/H222P} mice before (16 weeks) and after (20 weeks) the treatment with the MEK1/2 inhibitors (FIG. 3). The expression of atrial natriuretic factor was significantly decreased in AZD6244-treated and CI1040-treated mice. Even though it did not reach significance in PD0325901-treated mice, the expression of atrial natriuretic factor clearly showed a trend toward diminished expression compared to DMSO-treated mice (FIG. 23).

### 3) Effect of the MEK1/2 Inhibitors on the Expression of Myosin Chains

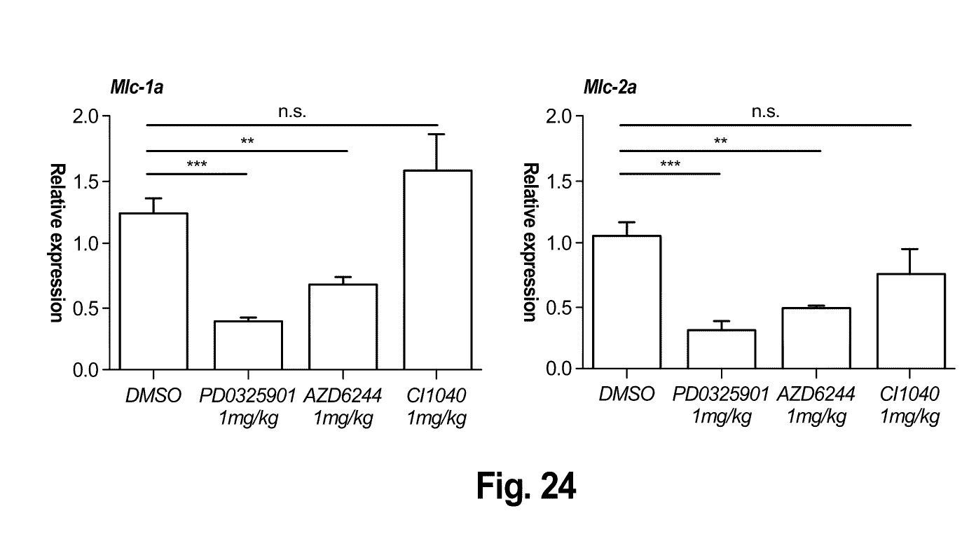

One of the features of dilated cardiomyopathy is the upregulation of genes involved in sarcomere organization also occurs [26,27]. We therefore assayed expression of Mlc-1a and Mlc-2a mRNA, encoding a cardiac isoform of myosin light chain in hearts from DMSO-treated Lmna^{H222P/H222P} mice and inhibitor-treated Lmna^{H222P/H222P} mice. In hearts from PD0325901-treated and AZD6244-treated Lmna^{H222P/H222P} mice, expression of Mlc-1a and Mlc-2a mRNAs were significantly decreased approximately 2-fold compared to hearts of DMSO-treated Lmna^{H222P/H222P} mice (FIG. 24). Hence, pharmacological inhibition of MEK1/2 signaling reversed molecular compensatory processes that occur in Lmna^{H222P/H222P} mice with cardiomyopathy.

### 4) Effect of the MEK1/2 Inhibitors on the Left Ventricle Dilatation and Contractility

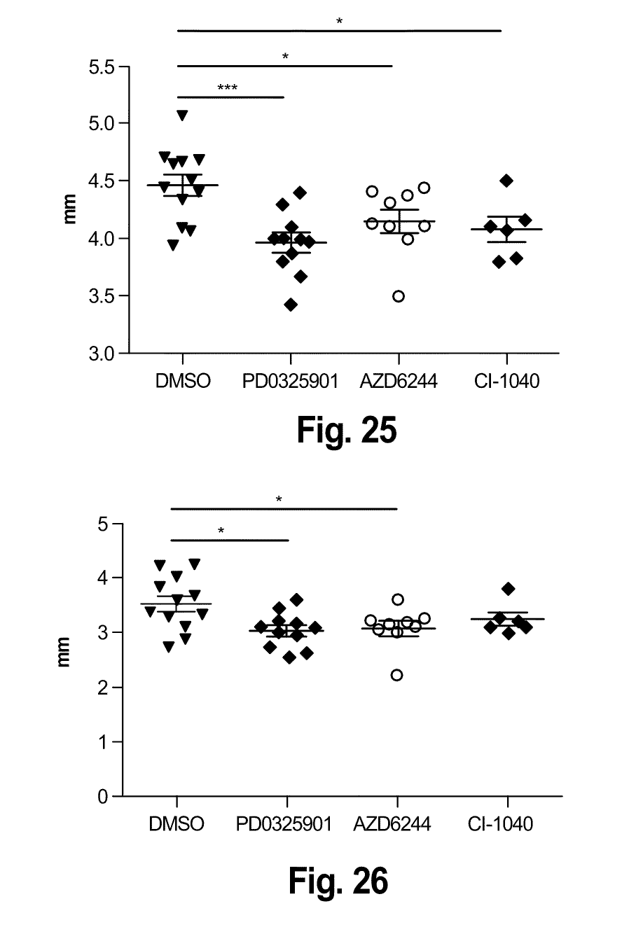

After 4 weeks of treatment with DMSO, PD0325901, AZD6244 and CI-1040, Lmna^{H222P/H222P} mice were anesthetized and cardiac dimensions and function measured by echocardiography. Lmna^{H222P/H222P} mice treated with PD0325901, AZD6244 and CI-1040 had significantly smaller left ventricular end diastolic diameter (LVEDD) compared to the DMSO-treated mice (FIG. 25).

Lmna^{H222P/H222P} mice treated with PD0325901 and AZD6244 also had significantly smaller left ventricular end systolic diameter (LVESD) compared to the DMSO-treated mice (FIG. 26).

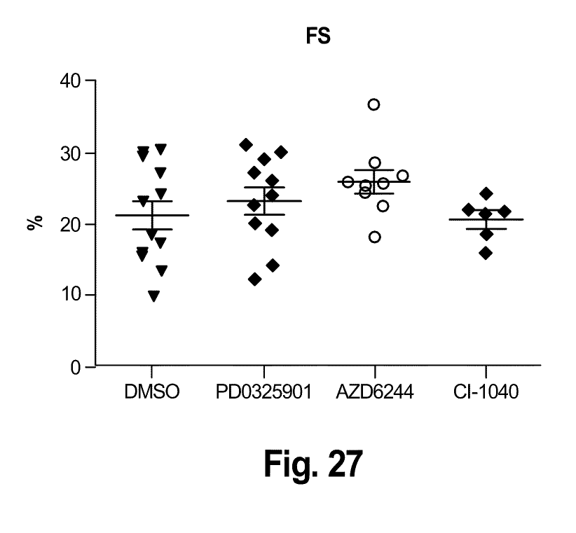

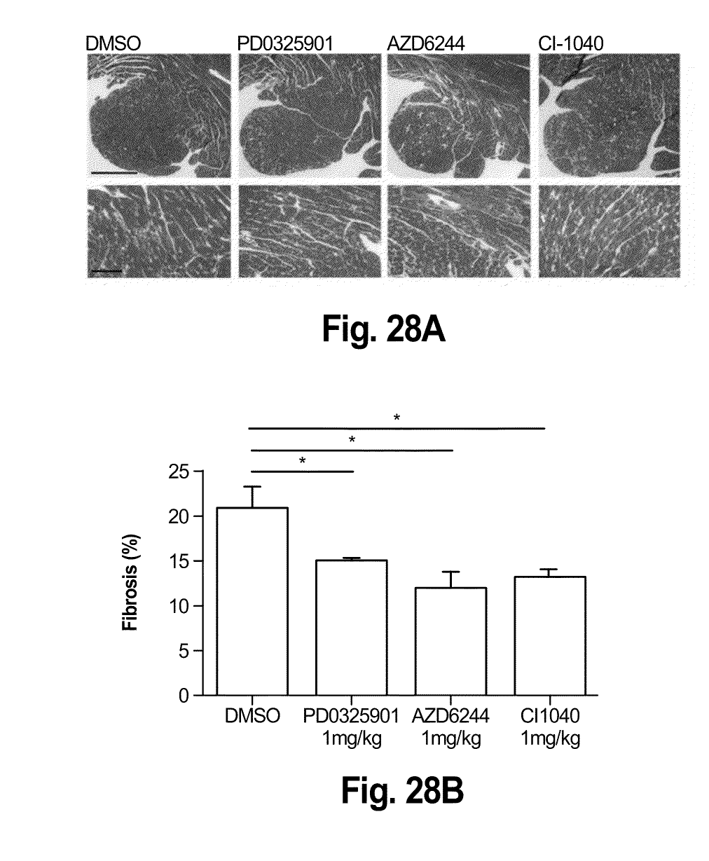

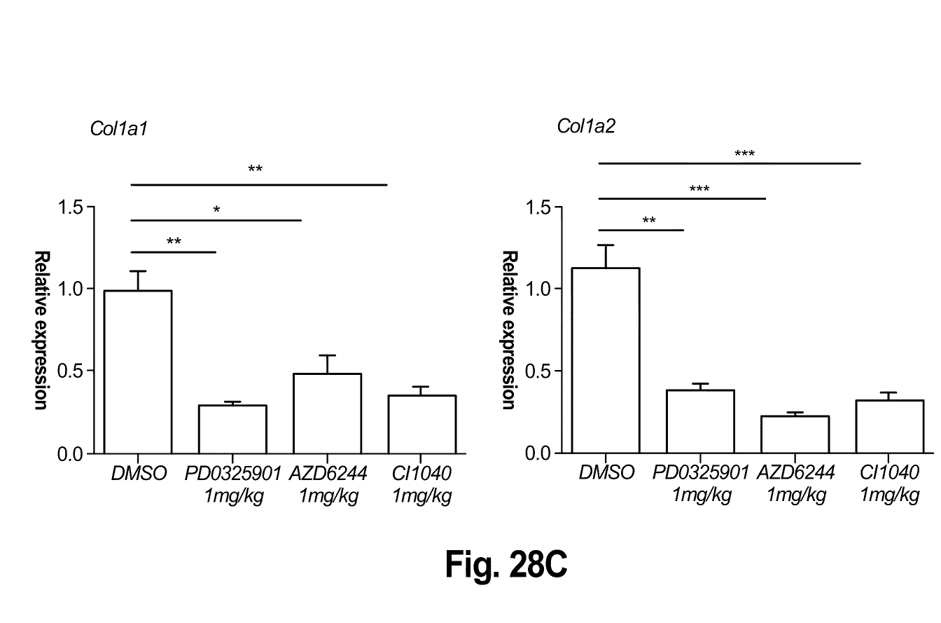

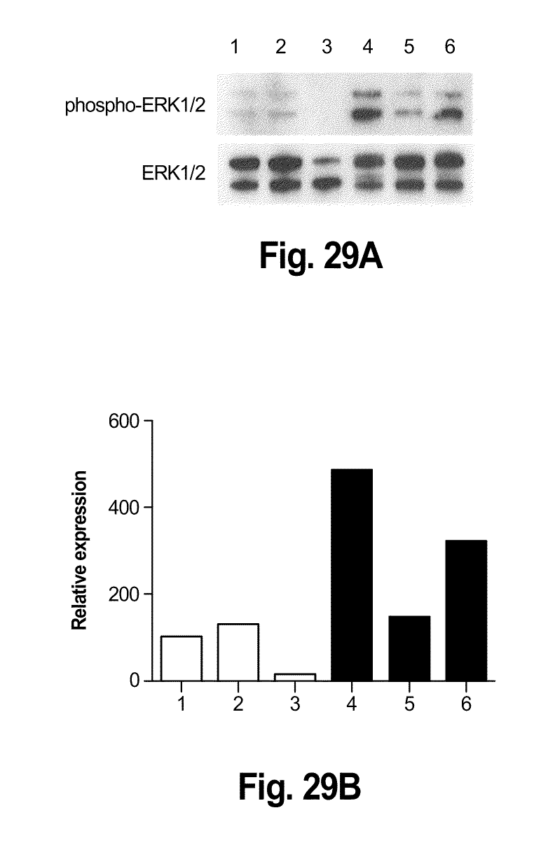

FS of Lmna^{H222P/H222P} mice treated with PD0325901 and AZD6244 were 23.23%±6.33%, and 25.97%±5.05%, respectively, compared to the DMSO-treated group (FIG. 27). Even though this parameter did not reach significance, we ran a power analysis and by adding few more mice to our study, it should become statistically significant. The FS was not improved in the CI-1040-treated Lmna^{H222P/H222P} _{mice.}

Overall, these results showed that PD0325901 and AZD6244 had positive effects on cardiac contractility when administered after cardiac dysfunction occurs in Lmna^{H222P/H222P} mice.

### 5) Effect of the MEK1/2 Inhibitors on Myocardial Fibrosis

Later-stage cardiomyopathy caused by LMNA mutations is characterized by myocardial fibrosis [28,29]. Gomori's trichrome staining of hearts from Lmna^{H222P/H222P} mice 20 weeks of age treated with DMSO had a significant increase in fibrosis compared to hearts from Lmna^{+/+} mice. In contrast, Lmna^{H222P/H222P} mice treated with PD0325901, AZD6244 and CI-1040 had a lower degree of cardiac fibrosis than DMSO-treated mice (FIG. 8A). We quantified the myocardial fibrotic area of each animal by determining the ratio of fibrotic tissue (blue stained with Gomori's trichrome) to the total tissue area in each micrograph (FIG. 8B). Hearts from DMSO-treated Lmna^{H222P/H222P} mice had 20.93%±2.45% fibrotic tissue per total surface examined (FIG. 8B). Systemic treatment with PD0325901, AZD6244 and CI-1040 significantly lowered the area of fibrotic tissue to 15.06%±0.28% (P<0.05), 11.90%±1.97% (P<0.05) and 13.10%±0.96% (P<0.05), respectively (FIG. 8B).

Excessive extracellular matrix, predominantly collagen proteins, defines fibrotic tissue. We therefore determined expression of genes encoding type I collagen (Colla1 and Cola2) using real-time RT-PCR. At 20 weeks of age, treatment with PD0325901, AZD6244 and CI-1040 significantly lowered the expression of Colla1 and Colla2 (FIG. 8C). These results demonstrated that Lmna^{H222P/H222P} mice treated with second-generation MEK1/2 inhibitors had decreased progression of myocardial fibrosis.

6) Level of Phospho-ERK1/2 Activation in Heart from Patients Carrying LMNA Mutations

We showed that inhibiting MEK1/2 signaling pathway in a mouse model of EDMD and CMD1A using small chemical entities improved the cardiac structure and function. The inhibitors had already been tested on humans for several types of cancer, and AZD6244 is currently under investigation for a phase II clinical trial. We re-profiled these molecules for the use in cardiomyopathy related to mutations in LMNA. Our data are leading the path for testing in humans.

We recently obtained cardiac biopsies from “control” (from NDRI-USA) and patients with LMNA mutations (from MyoBank-France) (Table 2).

TABLE 1
Listing of human cardiac biopsies

| | | | | | | LMNA |
|---|---|---|---|---|---|---|
| sample | age | sex | tissue | origin | diagnosis | mutation |
| 1 | 57 | M | right | NDRI | IC bleed | — |
| | | | ventricle | | | |
| 2 | 15 | F | right | NDRI | drug | — |
| | | | ventricle | | overdose | |
| 3 | 46 | M | right- | NDRI | end stage | — |
| | | | ventricle | | liver disease | |
| 4 | 23 | M | right- | MyoBank | EDMD | delK261 |
| | | | ventricle | | | |
| 5 | 47 | F | right- | MyoBank | FPLD + CHF | R60G |
| | | | ventricle | | | |
| 6 | 62 | F | right- | MyoBank | EDMD | cIVS9 + 1 g |
| | | | ventricle | | | sup a |

We showed that there was an increase of the phosphorylation of ERK1 and ERK2 in hearts from patients with LMNA mutation associated with EDMD, as shown by immunoblotting of proteins in tissue homogenates with antibodies against phosphorylated ERK1/2 and total ERK1/2 (FIG. 29).

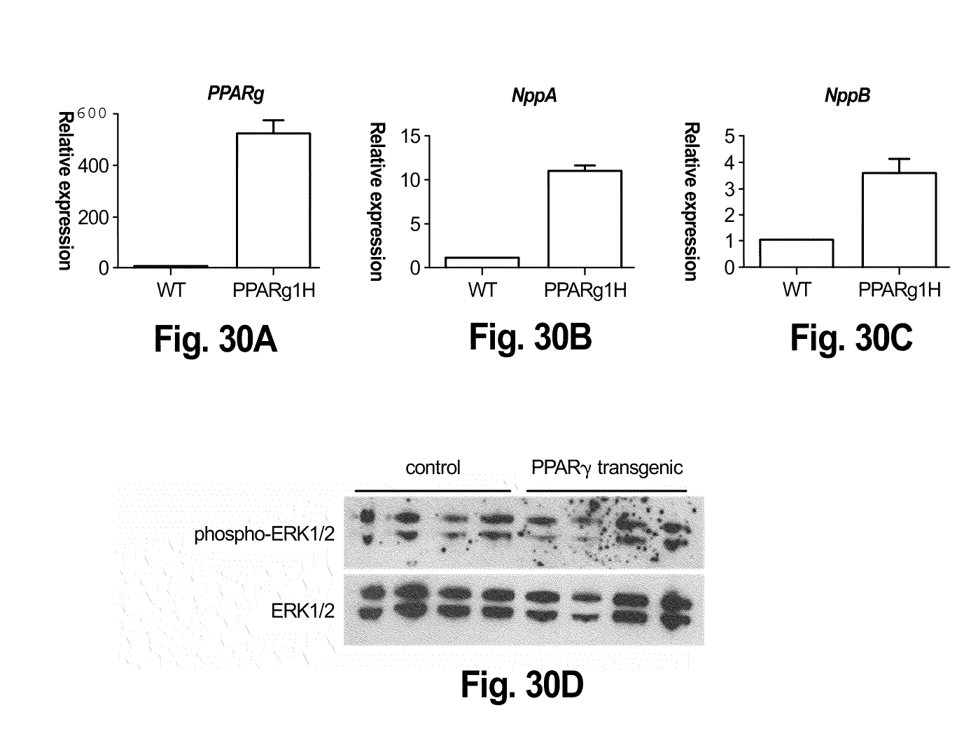

We note that there was no activation of ERK1/2 signaling in the heart from the patient with the LMNA mutation associated with FPLD, compared to the unaffected individuals. In this patient, the cardiac abnormality appeared to be secondary to the lipodystrophy and was not the typical cardiomyopathy observed in EDMD and CMD1A patients [30]. Along this line, we recently showed that there is no activation of phospho-ERK1/2 in mouse model of lipotoxic cardiomyopathy (transgenic mice expressing PPARγ [31]) (FIG. 30).

### Conclusion

Hence, this Example 6 shows that Lmna^{H222P/H222P} mice with cardiac dysfunction could lay the foundation for clinical trials of MEK1/2 inhibitors, currently being developed for cancer and inflammatory conditions in human subjects with cardiomyopathy caused by LMNA mutations.

### REFERENCE FOR EXAMPLE 6

1. Codd M B, Sugrue D D, Gersh B J, Melton L J. “Epidemiology of idiopathic dilated and hypertrophic cardiomyopathy. A population-based study in Olmsted County, Minn., 1975-1984.” Circulation 1989, 80:564-572.
2. Keeling P J, Gang G, Smith G, Seo H, Bent S E, Murday V, Caforio A L P, McKenna W J. “Familial dilated cardiomyopathy in the United kingdom.” Br Heart J 1995, 73:417-421.
3. Baig M K, Goldman J H, Caforio A L P, Coonar A S, Keeling P J, McKenna W J. “Familial dilated cardiomyopathy: cardiac abnormalities are common in asymptomatic relatives and may represent early disease.” J Am Coll Cardiol 1998, 31:195-201.
4. Gregori D, Rocco C, Miocic S, Mestroni L. “Estimating the frequency of familial dilated cardiomyopathy in the presence of misclassification errors.” J Appl Statistics 2001, 28:53-62.
5. Grunig E, Tasman J A, Kucherer H, Franz W, Kubler W, Katus H A. “Frequency and phenotypes of familial dilated cardiomyopathy.” J Am Coll Cardiol 1998, 31:186-194.
6. Cowan J, Li D, Gonzalez-Quintana J, Morales A, Hershberger R E. “Morphological analysis of 13 LMNA variants identified in a cohort of 324 unrelated patients with idiopathic or familial dilated cardiomyopathy.” Circ Cardiovasc Genet 2010 3:6-14.
7. van Tintelen J P, Hofstra R M, Katerberg H, Rossenbacker T, Wiesfeld A C, du Marchie Sarvaas G J, Wilde A A, van Langen I M, Nannenberg E A, van der Kooi A J, Kraak M, van Gelder I C, van Veldhuisen D J, Vos Y, van den Berg M P. “High yield of LMNA mutations in patients with dilated cardiomyopathy and/or conduction disease referred to cardiogenetics outpatient clinics.” Am Heart J 2007 154:1130-9.
8. Bilińska Z T, Sylvius N, Grzybowski J, Fidziańska A, Michalak E, Walczak E, Walski M, Bieganowska K, Szymaniak E, Kuśmierczyk-Droszcz B, Lubiszewska B, Wagner T, Tesson F, Ruzyllo W. “Dilated cardiomyopathy caused by LMNA mutations. Clinical and morphological studies.” Kardiol Pol 2006 64:812-9.
9. Vytopil M, Benedetti S, Ricci E, Galluzzi G, Dello Russo A, Merlini L, Boriani G, Gallina M, Morandi L, Politano L, Moggio M, Chiveri L, Hausmanova-Petrusewicz I, Ricotti R, Vohanka S, Toman J, Toniolo D. “Mutation analysis of the lamin A/C gene (LMNA) among patients with different cardiomuscular phenotypes.” J Med Genet 2003 40:132.
10. Taylor M R, Fain PR, Sinagra G, Robinson M L, Robertson A D, Carniel E, Di Lenarda A, Bohlmeyer T J, Ferguson D A, Brodsky G L, Boucek M M, Lascor J, Moss A C, Li W L, Stetler G L, Muntoni F, Bristow M R, Mestroni L. “Natural history of dilated cardiomyopathy due to lamin A/C gene mutations.” J Am Coll Cardiol 2003 41:771-80.
11. Grimm T, Janka M. “Emery-Dreifuss muscular dystrophy” Myology Second Edition 1994:1188-1191.
12. Muchir A, Shan J, Bonne G, Lehnart S E, Worman H J “Inhibition of extracellular signal-regulated kinase signaling to prevent cardiomyopathy caused by mutation in the gene encoding A-type lamins” Hum Mol Genet 2009; 18:241-247.
13. Wei W, Shan J, Bonne G, Worman H J, Muchir A “Pharmacological inhibition of c-Jun N-terminal kinase signaling prevents cardiomyopathy caused by mutation in LMNA gene” Biochim Biophys Acta 2010; 632-638.
14. Wang D, Boener S A, Winkler J D, LoRusso P M “Clinical experience of MEK inhibitors in cancer therapy” Bloch Biophys Acta 2007; 1248-1255.
15. Friday B B, Adjei A A “Advances in Targeting the Ras/Raf/MEK/Erk Mitogen-Activated Protein Kinase Cascade with MEK Inhibitors for Cancer Therapy” Clin Cancer Res 2008; 14: 342-6.
16. Margutti S, Laufer S A “Are MAP kinases drug targets? Yes, but difficult ones” Chem Med Chem 2007; 2:1116-40.
17. Messersmith W A, Hidalgo M, Carducci M, Eckhardt G S “Novel targets in solid tumors: MEK inhibitors” Clin Adv Hematol Oncol 2006; 4:831-836.
18. LoRusso P M, Adjei A A, Varterasian M, Gadgeel S, Reid J, Mitchell D Y, et al. “Phase I and pharmacodynamic study of the oral MEK inhibitor CI-1040 in patients with advanced malignancies” J Clin Oncol 2005; 23:5281-5293.
19. Rinehart J, Adjei A A, LoRusso P M, Waterhouse D, Hecht J R, Natale R B, et al. “Multicenter phase II study of the oral MEK inhibitor, CI-1040, in patients with advanced non-small-cell lung, breast, colon, and pancreatic cancer” J Clin Oncol 2004; 22:4456-4462.
20. LoRusso P, Krishnamurthi S, Rinehart J R, Nabell L, Croghan G, Varterasian M, et al. “A phase 1-2 clinical study of a second generation oral MEK inhibitor, PD0325901 in patients with advanced cancer” J Clin Oncol (Meet. Abstr.) 2005; 23:3011.
21. Menon S S, Whitfield L R, Sadis S, Meyer M B, Leopold J, LoRusso P M, et al. “Pharmacokinetics (PK) and pharmacodynamics (PD) of PD0325901, a second generation MEK inhibitor after multiple oral doses of PD0325901 to advanced cancer patients” J Clin Oncol (Meet. Abstr.) 2005; 23:3066.
22. Wallace E M, Lyssikatos J, Blake J F, Seo J, Yang H W, Yeh T C, et al. “Potent and selective mitogen-activated protein kinase kinase (MEK) 1,2 inhibitors. 1. 4-(4-bromo-2-fluorophenylamino)-1-methylpyridin-2(1H)-ones” J Med Chem 3006; 49:441-444.
23. Adjei A A, Cohen R B, Franklin W, Morris C, Wilson D, Molina J R, et al. “Phase I pharmacokinetic and pharmacodynamic study of the oral, small-molecule mitogen-activated protein kinase kinase ½ inhibitor AZD6244 (ARRY-142886) in patients with advances cancers” J Clin Oncol 2008; 26:2139-2146.
24. Yoshimine K, Horiuchi M, Suzuki S, Kobayashi K, Abdul J M, Masuda M, Tomomura M, Ogawa Y, Itoh H, Nakao K, Osame M, Saheki T. Altered expression of atrial natriuretic peptide and contractile protein genes in hypertrophied ventricles of JVS mice with systemic carnitine deficiency. J Mol Cell Cardiol. 1997; 29:571-578.
25. Takahashi T, Allen P D, Izumo S. Expression of A-, B-, and C-type natriuretic peptide genes in failing and developing human ventricles. Correlation with expression of the Ca(2+)-ATPase gene. Circ Res. 1992; 71:9-17.
26. Hwang J J, Allen P D, Tseng G C, Lam C W, Fananapazir L, Dzau V J, Liew C C. Microarray gene expression profiles in dilated and hypertrophic cardiomyopathic end-stage heart failure. Physiol Genomics. 2002; 10:31-44.
27. Yung C K, Halperin V L, Tomaselli G F, Winslow R L. Gene expression profiles in end-stage human idiopathic dilated cardiomyopathy: altered expression of apoptotic and cytoskeletal genes. Genomics. 2004; 83:281-297.
28. Van Tintelen P J, Tio R A, Kerstjens-Frederikse W S, van Berlo J H, Boven L G, Suurmeijer A J H, White S J, den Dunnen J T, to Meerman G J, Vos Y J, van der Hout A H, Osinga J, van den Berg M P, van Verhuisen D J, Buys C H C M, Hofstra R M W, Pinto Y M. Severe myocardial fibrosis caused by a deletion of the 5′ end of the lamin A/C gene. J Am Coll Cardiol. 2007; 49:2430-2439.
29. Raman S V, Sparks E A, Baker P M, McCarthy B, Wooley C F. Mid-myocardial fibrosis by cardiac magnetic resonance in patients with lamin A/C cardiomyopathy: possible substrate for diastolic dysfunction. J Cardiovac Magn Res. 2007; 9:907-913.
30. Van der Koii A J, Bonne G, Eymard B, Duboc D, Talim B, van der Valk M, Reiss P, Richard P, Demay L, Merlini L, Schwartz K, Busch H F M, de Visser M. Lamin A/C mutations with lipodystrophy, cardiac abnormalities, and muscular dystrophy. Neurology. 2002; 59:620-623.
31. Son N H, Park T S, Yamashita H, Yokoyama M, Huggins L A, Okajima K, Homma S, Szabolcs M, Huang L S, Goldberg I. Cardiomyocyte expression of PPARg leads to cardiac dysfunction in mice. J Clin Invest. 2007; 117:2791-2801.

### Example 7

### Mitogen Activated Protein Kinase Inhibitors Improve Heart Function and Prevent Fibrosis in Cardiomyopathy Caused by Mutation in Lamin A/C Gene

The numbers in parentheses below refer to the references in the numbered list that follows this Example 7.

In Example 7, to determine if pharmacological inhibitors of ERK and JNK signaling were clinically useful to treat cardiomyopathy caused by LMNA mutation, we administered them to Lmna^{H222P/H222P} mice after they developed left ventricular dilatation and decreased ejection fraction. Lmna^{H222P/H222P} mice were treated with ERK and JNK signaling inhibitors from 16 to 20 or, in pilot experiments, 19 to 24 weeks of age. The inhibitors blocked increased expression of RNAs encoding natriuretic peptide precursors and proteins involved in sarcomere architecture that occurred in placebo-treated mice. Echocardiography and histological analysis demonstrated that treatment prevented left ventricular end systolic dilatation, increased ejection fraction and decreased myocardial fibrosis. Thus, this Example showed that inhibitors of ERK and JNK signaling can be used to treat humans with cardiomyopathy caused by LMNA mutations.

Dilated cardiomyopathy is characterized by ventricular dilatation and impaired systolic function with 20% to 48% of cases familial (1). Mutations in LMNA encoding A-type nuclear lamins have been shown to cause a several human diseases (2) with at least 3 having dilated cardiomyopathy as a predominant feature: autosomal Emery-Dreifuss muscular dystrophy (3), limb girdle muscular dystrophy type 1B (4) and dilated cardiomyopathy type 1A (5). Given the phenotypic overlap of these disorders, they can be described as LMNA dilated cardiomyopathy with variable skeletal muscle involvement (6). LMNA mutations appear to be responsible for approximately 8% of familial cardiomyopathies (7-10). The onset of symptoms in LMNA cardiomyopathy is variable, ranging from the first to sixth decade of life and occurring most frequently in the third decade (7-11). It has a natural history more aggressive than most other familial cardiomyopathies, with high rates of arrhythmias leading to sudden death and advanced heart failure necessitating cardiac transplantation (7,11,12).

To identify targets to treat cardiomyopathy caused by LMNA mutation, we have been examining cellular signaling pathways in hearts of Lmna H222P knock in mice, a model of the human disease. Male Lmna^{H222P/H222P} mice develop left ventricular (LV) dilatation and depressed contractile function starting at approximately 8-10 weeks of age and invariably develop LV dilatation and decreased cardiac contractility at 16 weeks (13). We have shown abnormal activation of the extracellular signal-regulated kinase (ERK) and the c-Jun N-terminal kinase (INK) branches of the mitogen-activated protein kinase (MAPK) signaling cascade in hearts of Lmna H222P knock in mice prior to the onset of clinically detectable cardiomyopathy (14). We have also shown that lamin A variants that cause cardiomyopathy activate ERK and JNK when expressed in cultured cells (14). Based on these results, we hypothesized that activation of ERK and JNK plays a primary pathogenic role in the development of cardiomyopathy. Our recent work has shown that small molecule inhibitors of ERK and JNK signaling administered to male Lmna^{H222P/H222P} mice prior to the onset of detectable cardiomyopathy prevented LV dilatation and decreases in cardiac ejection fraction (EF) at an age when placebo-treated mice had significant abnormalities in these parameters (15,16).

A critical question relevant to treatment of human subjects with ERK and JNK inhibitors regards their effectiveness after the onset of cardiac dysfunction. It would be impractical to use such drugs as prophylactic treatment in asymptomatic humans with LMNA mutations, especially given the variable age of onset, usually in adulthood. To help answer this question, we conducted this study to determine if inhibitors of ERK and JNK signaling would be beneficial in Lmna^{H222P/H222P} mice after LV dilatation and decreased cardiac EF have already occurred.

### Materials and Methods

### Mice

Lmna^{H222P/H222P} mice were generated and genotyped as previously described (13). Genotyping was performed by polymerase chain reaction (PCR) of genomic tail DNA using oligonucleotides 5′-cagccatcacctctcctttg-3′ and 5′-agcaccagggagaggacagg-3′. Mice were fed a chow diet and housed in a disease-free barrier facility with 12 h/12 h light/dark cycles. The Institutional Animal Care and Use Committee at the Columbia University Medical Center approved the use of vertebrate animals and the study protocol.

### JNK and MEK Inhibitors

SP600125, an anthrapyrazolone inhibitor of JNK, and PD98059, a 2′-amino-3′-methoxyflavone MAPK/ERK kinase (MEK) ½ inhibitor, (Calbiochem) were dissolved in dimethyl sulfoxide (DMSO) (Sigma) at a concentration of 1 mg/ml and were delivered to a dose of 3 mg/kg/day for 5 days a week. The placebo control consisted of DMSO alone and was delivered in the same volume. Placebo and inhibitors were administered by intraperitoneal injection using a 27⅝-gauge syringe. Treatments were started when mice were 16 weeks of age and continued until 20 weeks of age or when the mice were 19 weeks and continued until 24 weeks of age.

### Thansthoracic Echocardiography

At 20 weeks or 24 weeks of age, mice were anesthetized with 1.5% isoflurane in O₂ and placed on a heating pad (37° C.). Cardiac function was assessed by echocardiography with a Visualsonics Vevo 770 ultrasound with a 30 MHz transducer applied to the chest wall. Cardiac ventricular dimensions and fractional shortening (FS) were measured in 2D mode and M-mode three times in a modified short axis view for the number of animals indicated. A “blinded” echocardiographer (J.S.), unaware of the genotype or treatment, performed the examinations.

### Histopathological Analysis

Mice were sacrificed at 20 weeks of age, and freshly removed hearts were fixed in 4% formaldehyde for 48 hours, embedded in paraffin, sectioned at 5 μm and stained with hematoxylin, eosin and Gomori's trichrome and Syrius Red. Representative stained sections were photographed using a Microphot SA (Nikon) light microscope attached to a Spot RT Slide camera (Diagnostic Instruments) with a 10× objective. Images were processed using Adobe Photoshop CS (Adobe Systems).

### Immunoblot Analysis

Proteins were loaded on 10% SDS gels and electrotransferred on a 0.45 mM pore size nitrocellulose membrane (Invitrogen). We used antibodies directed against phosphorylated ERK1/2 (Cell Signaling, 1:1,000), total ERK1/2 (Santa Cruz, 1:2,000), phosphorylated JNK (Cell Signaling, 1:1,000) and total JNK (Santa Cruz, 1:200). The secondary antibodies were coupled to horseradish-peroxydase (Amersham). Recognized proteins were visualized by enhanced chemiluminescence (SuperSignal® West Pico chemiluminescent substrate, Thermo Scientific).

Quantitative mRNA Analyses

Real-time reverse transcription-PCR (RT-PCR) was used to quantify tissue RNA levels. Total RNA was extracted from cardiac ventricles of mice using the Rneasy isolation kit (Qiagen) as previously described. cDNA was synthesized using Superscript first strand synthesis system according to the manufacturer's instructions (Invitrogen) on total RNA. For each replicate in each experiment, RNA from tissue samples of different animals was used. Primers were designed correspond to mouse RNA sequences using Primer3 (http://frodo.wi.mit.edu/cgi-bin/primer3/primer3_www.cgi) for Nppa (forward 5′-gcttccaggccatattggag-3′ [SEQ ID NO: 4], reverse 5′-ccctgcttcctcagtctgct-3′ [SEQ ID NO: 5]), Nppb (forward 5′-ggaccaaggcctcacaaaag-3′ [SEQ ID NO: 6], reverse 5′-tacagcccaaacgactgacg-3′ [SEQ ID NO: 7]), Mlc-2a (forward 5′-tcaaggaagccttcagctgc-3′ [SEQ ID NO: 8], reverse 5′-cggaacacttaccctcccg-3′ [SEQ ID NO: 9]), Colla1 (forward 5′-agacggacagtactggatcg-3′ [SEQ ID NO: 10], reverse 5′-gcttcttttccttggggttc-3′ [SEQ ID NO: 11]), Colla2 (forward 5′-ccgtgcttctcagaacatca-3′ [SEQ ID NO: 12], reverse 5′-gagcagccatcgactaggac-3′ [SEQ ID NO: 13]) and Fn1 (forward 5′-aatggaaaaggggaatggac-3′ [SEQ ID NO: 14], reverse 5′-ctcggttgtccttcttgctc-3′ [SEQ ID NO: 15]). The real-time RT-PCR reaction contained iQ SYBR green super mix (Bio-Rad), 200 nM of each primer, and 0.2 μl of template in a 25 μl reaction volume. Amplification was carried out using the MyiQ Single-Color Real-Time PCR Detection System (Bio-Rad) with an initial denaturation at 95° C. for 2 mM followed by 50 cycles at 95° C. for 30 s and 62° C. for 30 s. Relative levels of mRNA expression were calculated using the ΔΔCT method (2). Individual expression values were normalized by comparison with Gapdh mRNA (forward 5′-tgcaccaccaactgcttag-3′ [SEQ ID NO: 16], reverse 5′-ggatgcagggatgatgttc-3′ [SEQ ID NO: 17]).

### Statistical Analysis

A commercial software package (Prism Software Inc.) was used to perform all statistical analyses. For most experiments, significance was determined using parametric test (Welch's one-way analysis of variance [ANOVA]) with a value of P<0.05 considered significant. To validate all results, nonparametric test (Mann-Whitney) was performed and concordance checked. To determine significant differences in the cardiomyocyte nuclei length between groups of animals, we calculated average nuclear lengths for each mouse and used a parametric test (Welch's one-way ANOVA) with a value of P<0.05 considered significant. To determine significant differences between groups of animals analyzed by echocardiography, we used one-way ANOVA. We then used a Tukey adjustment for post hoc multiple comparisons (5% type I error) to determine which groups were significantly different. Comparisons of the echocardiographic parameters between the different groups of mice were also performed using a Student unpaired t-test and shown are in Table 11. The normality of residuals was validated using D'Agostino and Pearson tests. To validate all results, nonparametric tests (Kruskal-Wallis and Mann-Whitney) were performed and concordance checked. All data are presented as means±standard errors of means (SEM).

Lmna^{H222P/H222P} mice were generated and genotyped using polymerase chain reaction (PCR) primers as described (13). Drugs were dissolved in dimethyl sulfoxide (DMSO) are delivered into the peritoneal cavity by injection at 3 mg/kg/day for 5 days a week. Equal volumes of DMSO were administered as placebo. Cardiac structure and contractility were assessed by echocardiography. Representative stained cardiac sections were photographed using a Microphot SA (Nikon) light microscope attached to a Spot RT Slide camera (Diagnostic Instruments) with a 10× objective. Images were processed using Adobe Photoshop CS (Adobe Systems). RNA transcripts measured using real-time reverse transcription-polymerase chain reaction (RT-PCR) were quantified using iQ SYBR green super mix (Bio-Rad). Statistical comparisons were made using an unpaired Student's t-test or a one-way analysis of variance with the Tukey post hoc test to evaluate the significance of differences between means.

### Results

### Treatment of Lmna^{H222P/H222P} Mice

Treatment with a MAPK/ERK kinase (MEK) ½ inhibitor, which inhibits activation of ERK, or a JNK inhibitor improved cardiac structure and function in Lmna^{H222P/H222P} mice when the compounds were administered after these parameters were significantly abnormal. We assigned male Lmna^{H222P/H222P} mice 16 weeks of age to 3 different treatment arms (placebo DMSO, n=28; MEK1/2 inhibitor PD98059, n=22; JNK inhibitor SP600125, n=29) and examined parameters of cardiac structure and function at 20 weeks of age, after 4 weeks treatment. At 16 weeks, male Lmna^{H222P/H222P} mice are known to have markedly increased LV end diastolic diameter (LVEDD) and LV end systolic diameter (LVESD) compared to Lmna^{+/+} mice (13,15,16). Lmna^{H222P/H222P} mice also have depressed cardiac contractility, with fractional shortening (FS) decreased by 20%-40% compared to Lmna^{+/+} mice (13,15). Myocardial fibrosis occurs in Lmna^{H222P/H222P} mice at 16 weeks of age (16). At 20 weeks, LVEDD and LVESD increase further in Lmna^{H222P/H222P} mice and cardiac contractility also progressively deteriorates (16). During the 4-week treatment protocol, 6 mice in the DMSO group, 3 in the PD98059 group and 3 in the SP600125 group died prior to reaching 20 weeks of age for evaluation.

### Effect of PD98059 and SP600125 on ERK and JNK Signaling

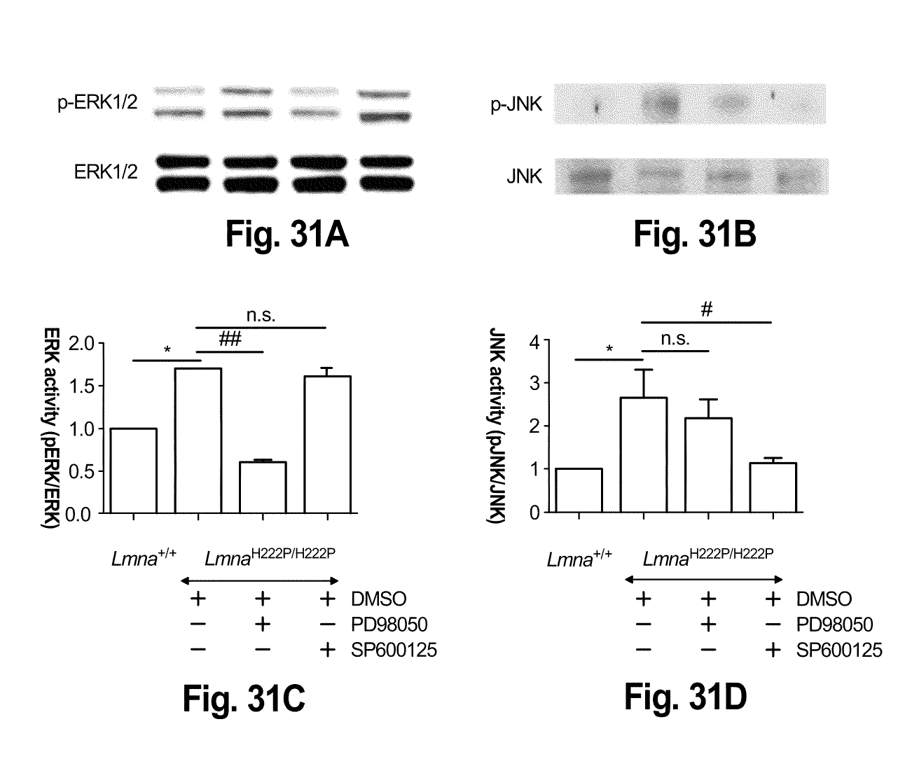

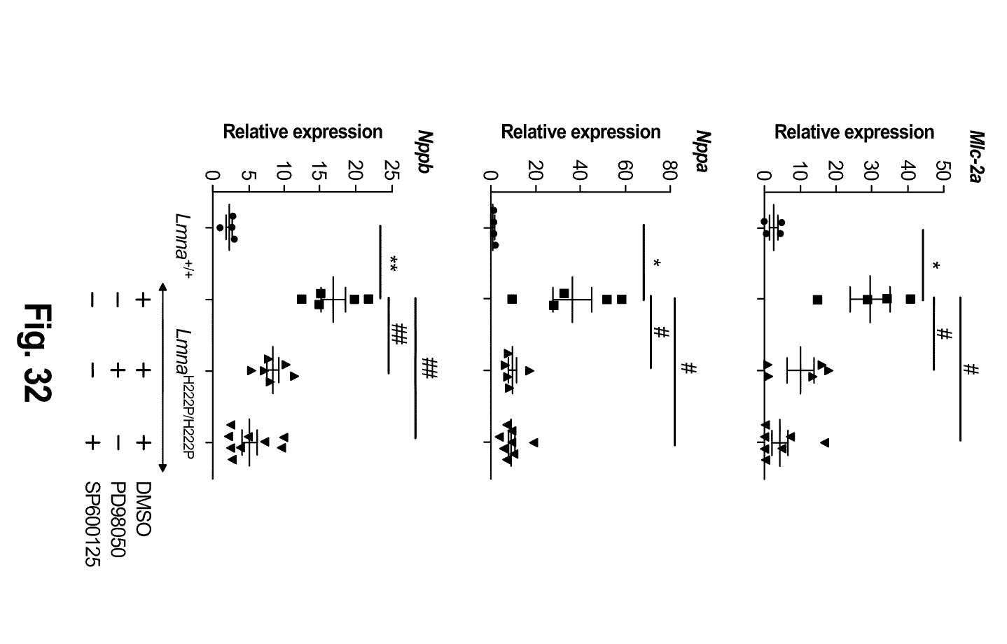

Systemic administration of the MEK1/2 inhibitor, PD98059, and the JNK inhibitor, SP600125, to Lmna^{H222P/H222P} mice from 16 to 20 weeks of age partially blocked the phosphorylation of ERK1/2 (FIG. 31A) and JNK (FIG. 31B), respectively, in hearts. At 3 mg/kg/day, PD98059 was highly selective for blocking ERK signaling, as phosphorylation of JNK was not significantly inhibited (FIG. 31A). At 3 mg/kg/day, SP600125 was specific of the JNK signaling, as phosphorylation of ERK1/2 was not significantly inhibited (FIG. 31B).

### Effect of the PD98059 and SP600125 on Cardiac Expression of Natriuretic Peptides and Myosin Light Chain

One of the features of dilated cardiomyopathy is the upregulation of cardiac hormones such as natriuretic peptides as a compensatory mechanism to maintain cardiac output (17,18). Upregulation of genes involved in sarcomere organization also occurs (19,20). We therefore assayed expression of Mlc-2a mRNA, encoding a cardiac isoform of myosin light chain, and NppA and NppB mRNAs, encoding natriuretic peptides precursors in hearts from Lmna^{+/+} mice, DMSO-treated Lmna^{H222P/H222P} mice and inhibitor-treated Lmna^{H222P/H222P} mice (FIG. 32).

In hearts from DMSO-treated Lmna^{H222P/H222P} mice, expression of Mlc-2a mRNA was significantly increased approximately 30-fold compared to hearts of Lmna^{-/+} mice (FIG. 32). Similarly, in hearts from Lmna^{H222P/H222P} mice, NppA and NppB mRNA levels showed significant 36-fold and 17-fold increases in expression compared to hearts of Lmna^{-/+} mice (FIG. 32). Treatment of Lmna^{H222P/H222P} mice with PD98059 or SP600125 significantly decreased the expression of Mlc-2a, NppA and NppB mRNAs at 20 weeks of age (FIG. 32). Hence, pharmacological inhibition of ERK or JNK signaling reversed molecular compensatory processes that occur in Lmna^{H222P/H222P} mice with cardiomyopathy.

### Effect of PD98059 and SP600125 on LV Dilatation and Contractility in Lmna^{H222P/H222P} Mice

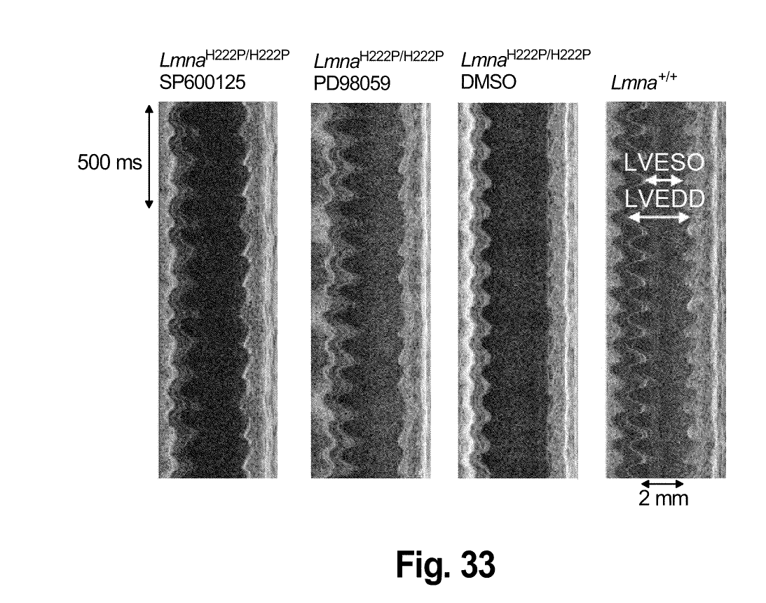

After 4 weeks of treatment with DMSO, PD98059 or SP600125, Lmna^{H222P/H222P} mice were anesthetized and cardiac dimensions and function measured by echocardiography. M-mode transthoracic echocardiography showed increased LVEDD and LVESD in Lmna^{H222P/H222P} mice treated with DMSO compared to Lmna^{+/+} mice (FIG. 33). Lmna^{H222P/H222P} mice treated with PD98059 and SP600125 had significantly smaller LVESD compared to the DMSO-treated mice (FIG. 33). FS and EF were reduced in Lmna^{H222P/H222P} mice compared to Lmna^{+/+} mice but increased in the Lmna^{H222P/H222P} mice treated with PD98059 or SP600125.

Table 11 below shows the composite echocardiographic data for the 3 treatment arms for Lmna^{H222P/H222P} mice and Lmna^{+/+} mice for comparison. Compared to Lmna^{+/+} mice, Lmna^{H222P/H222P} mice treated with DMSO had significantly increased LVEDD and LVESD. The EF of DMSO-treated male Lmna^{H222P/H222P} mice at 20 weeks was 53.87%±2.58%, which was decreased by 28% compared to Lmna^{+/+} mice. Lmna^{H222P/H222P} mice treated with PD98059 or SP600125 had a statistically significant reduction in the LVESD compared to mice treated with DMSO; however, LVEDD was not significantly different. Lmna^{H222P/H222P} mice treated with PD98059 had an EF of 65.46%±2.64%, an increase of approximately 22% (P<0.005) compared to the DMSO-treated group. EF of Lmna^{H222P/H222P} mice treated with SP600125 was 61.88%±1.66%, an increase of approximately 15% (P<0.005) compared to the DMSO-treated group. Overall, these results showed that PD98059 and SP600125 have

TABLE 11
Echocardiographic data at 20 weeks of age for Lmna^{+/+} mice and
Lmna^{H222P/H222P} mice treated with DMSO placebo or treated with SP600125 or PD98059

| | | | LVEDD | LVESD | | |
|---|---|---|---|---|---|---|
| Genotype (Treatment Group) | n | HR | (mm) | (mm) | EF (%) | FS (%) |
| Lmna^{+/+} | 12 | 400 | 3.50 ± 0.06 | 2.07 ± 0.08 | 73.21 ± 1.17 | 41.71 ± 1.01 |
| Lmna^{H222P/H222P} (DMSO) | 22 | 372 | 3.87 ± 0.11 * | 3.00 ± 0.13 *** | 53.87 ± 2.58 *** | 27.86 ± 1.54 *** |
| Lmna^{H222P/H222P} (PD98059) | 19 | 350 | 3.55 ± 0.11 | 2.41 ± 0.11 ^{‡‡‡} | 65.46 ± 2.64 ^{‡‡} | 35.91 ± 1.88 ^{‡‡} |
| Lmna^{H222P/H222P} (SP600125) | 26 | 363 | 3.73 ± 0.08 | 2.67 ± 0.10 ^{‡} | 61.88 ± 1.66 ^{‡‡} | 33.11 ± 1.16 ^{‡‡} |

Values are means ± SEM.
HR indicates heart rate in beats per minute.
Comparison between DMSO-treated Lmna^{H222P/H222P} and Lmna^{+/+} mice:
* P < 0.05,
*** P < 0.0005.
Comparison between SP600125-treated and DMSO-treated Lmna^{H222P/H222P}:
^{‡} P < 0.05,
^{‡‡} P < 0.005,
^{‡‡‡} P < 0.0005.

positive effects on cardiac contractility when administered after cardiac dysfunction occurs in Lmna^{H222P/H222P} mice.

### Effect of PD98059 and SP600125 on Myocardial Fibrosis in Lmna^{H222P/H222P} Mice

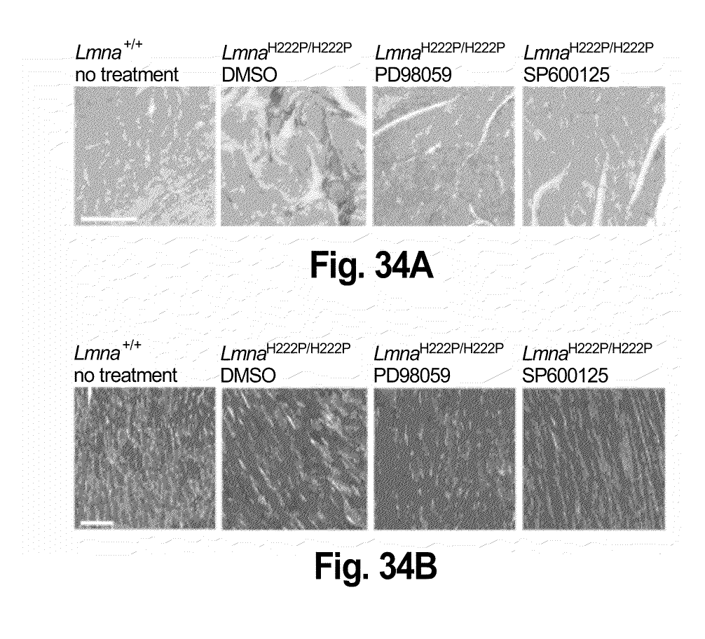

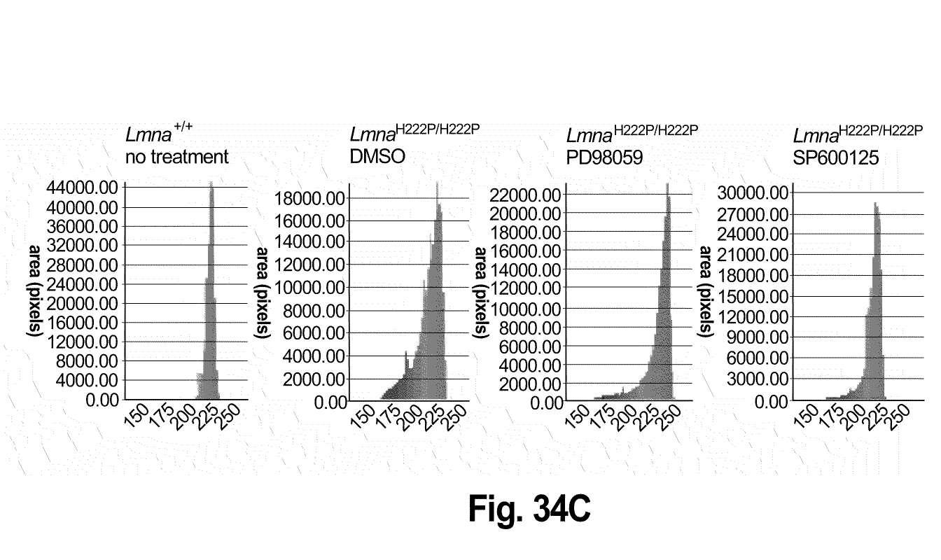

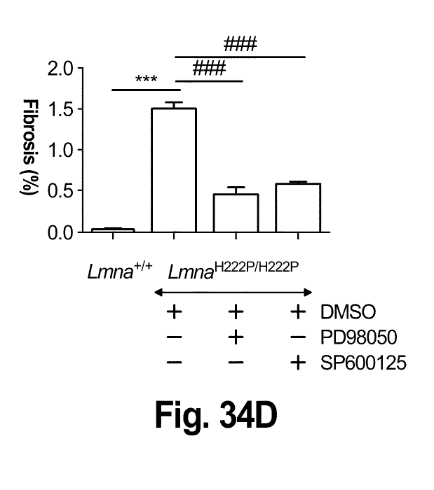

Later-stage cardiomyopathy caused by LMNA mutations is characterized by myocardial fibrosis (21,22). Sirius Red and Gomori's trichrome staining of hearts from Lmna^{H222P/H222P} mice 20 weeks of age treated with DMSO had a significant increase in fibrosis compared to hearts from Lmna^{+/+} mice (FIGS. 34A-B). In contrast, Lmna^{H222P/H222P} mice treated with PD98059 or SP600125 had a lower degree of cardiac fibrosis than DMSO-treated mice (FIGS. 34A-B). We quantified the myocardial fibrotic area of each animal by determining the ratio of fibrotic tissue (blue stained with Gomori's trichrome) to the total tissue area in each micrograph (FIG. 34C). Hearts from DMSO-treated Lmna^{H222P/H222P} mice had 15.01±0.9% fibrotic tissue per total surface examined (FIG. 34D). Systemic treatment with PD98059 or SP600125 significantly lowered the area of fibrotic tissue to 4.48%±1% (P<0.0005) and 5.86%±0.4% (P<0.0005), respectively (FIG. 34D).

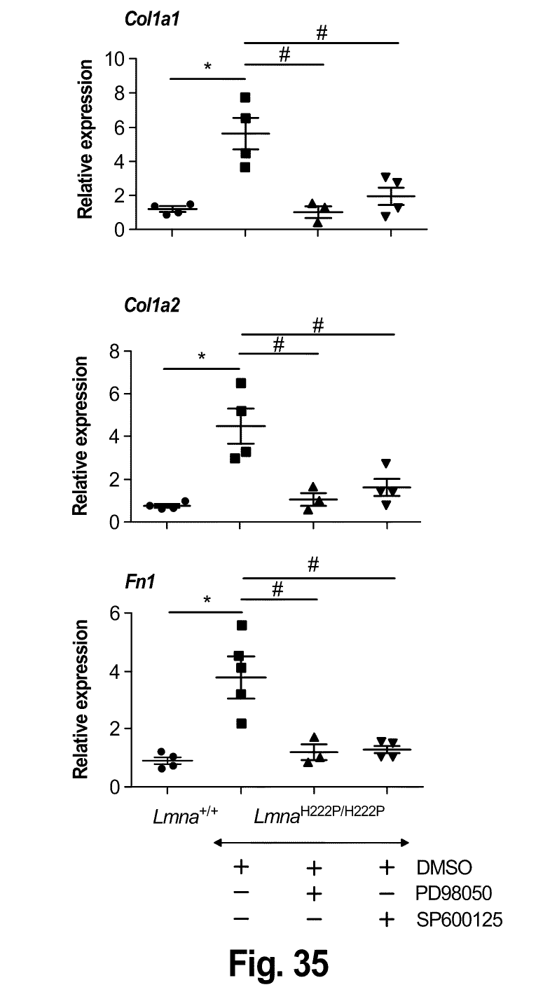

Excessive extracellular matrix, predominantly collagen proteins, defines fibrotic tissue. We therefore determined expression of genes encoding a protein of the extracellular matrix (Fn1 encoding fibronectin) and genes encoding type I collagen (Colla1 and Cola2) using real-time RT-PCR. At 20 weeks of age, hearts from Lmna^{H222P/H222P} mice treated with DMSO had a 5-fold increase of Colla1, a 4-fold increase of Cola2 and a 4-fold increase of Fn1 mRNAs compared to hearts from Lmna^{+/+} mice (FIG. 35). Treatment with PD98059 and SP600125 significantly lowered the expression of Colla1, Colla2 and Fn1 (FIG. 35). These results demonstrated that Lmna^{H222P/H222P} mice treated with either MEK1/2 or JNK inhibitors had decreased progression of myocardial fibrosis.

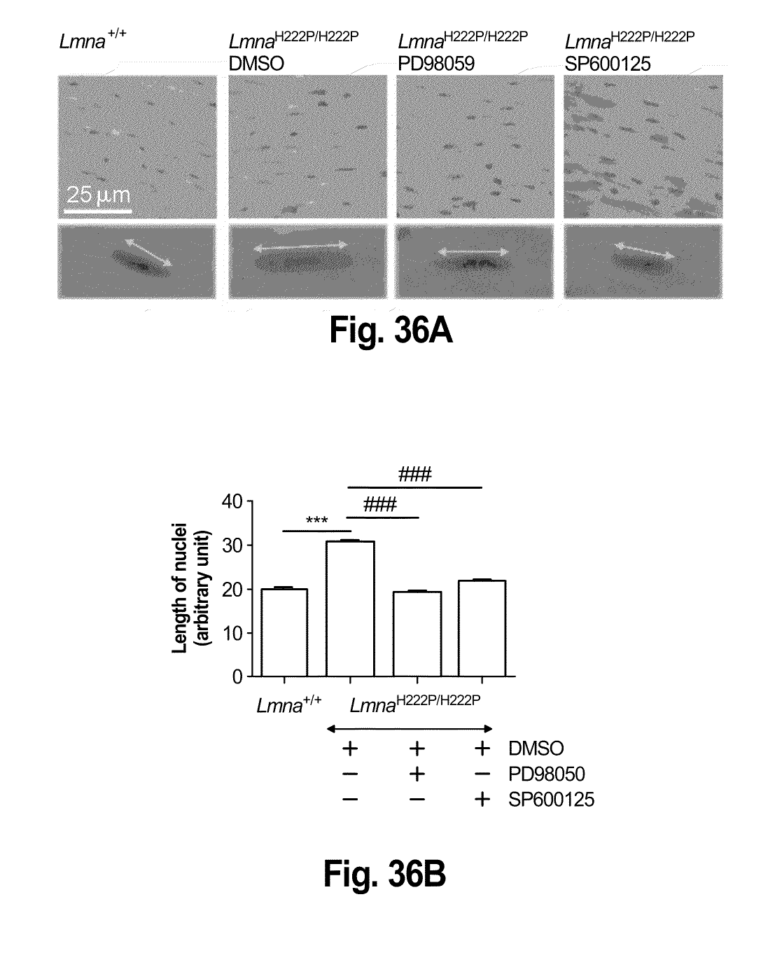

Effect of PD98059 and SP600125 on Nuclear Shape in Cardiomyocytes in Lmna^{H222P/H222P} Mice We have reported abnormal elongation of nuclei in cardiomyocytes of Lmna^{H222P/H222P} mice (15,16). Nuclei in cardiomyocytes in hearts from Lmna^{H222P/H222P} mice treated with DMSO were elongated compared to those in Lmna^{+/+} mice (FIG. 36A). Nuclei of cardiomyocytes in hearts of Lmna^{H222P/H222P} mice treated with PD98059 or SP600125 Lmna^{H222P/H222P} mice had an overall shape that was more “rounded” than those in hearts of mice treated with DMSO (FIG. 36A). Mean length of cardiomyocyte nuclei in hearts of Lmna^{H222P/H222P} mice treated with DMSO was significantly longer than in hearts from Lmna^{+/+} mice (P<0.0005) (FIG. 36B). The mean lengths of nuclei in cardiomyocytes in hearts from Lmna^{H222P/H222P} mice treated with PD98059 or SP600125 were significantly shorter than the in hearts of mice in the DMSO-treated group (P<0.0005) (FIG. 36B). Similar nuclear elongation has also been reported in Lmna knockout mice, suggesting a role of lamins in determining nuclear shape in cardiomyocytes (23,24).

Pilot Study of PD98059 and SP600125 to Treat More Advanced Heart Disease in Lmna^{H222P/H222P} mice

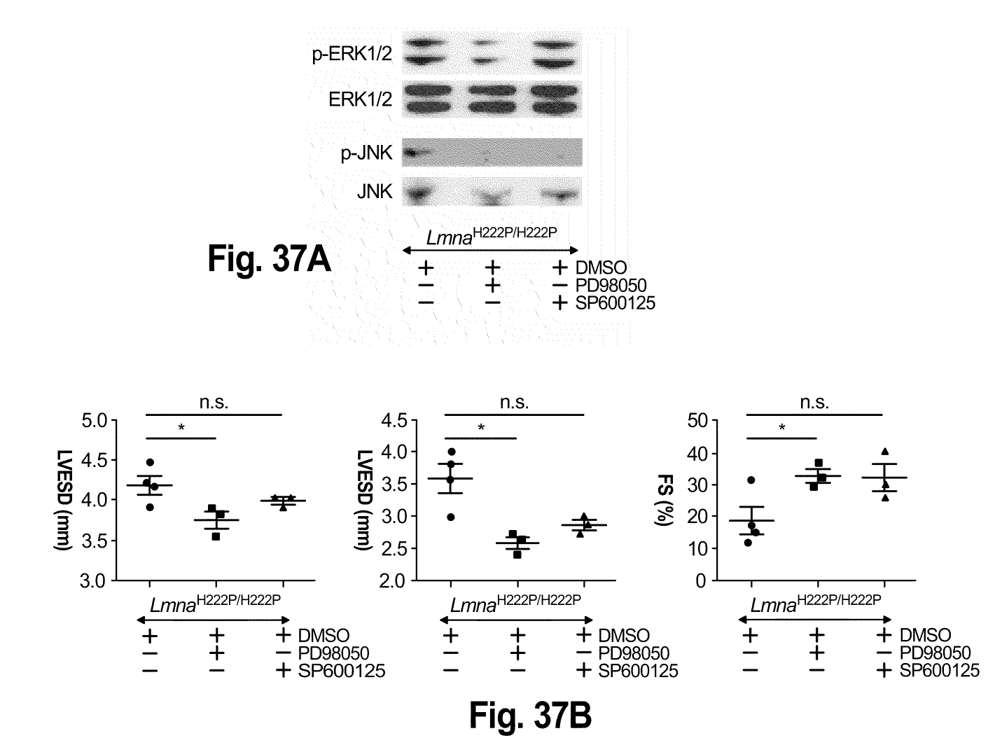

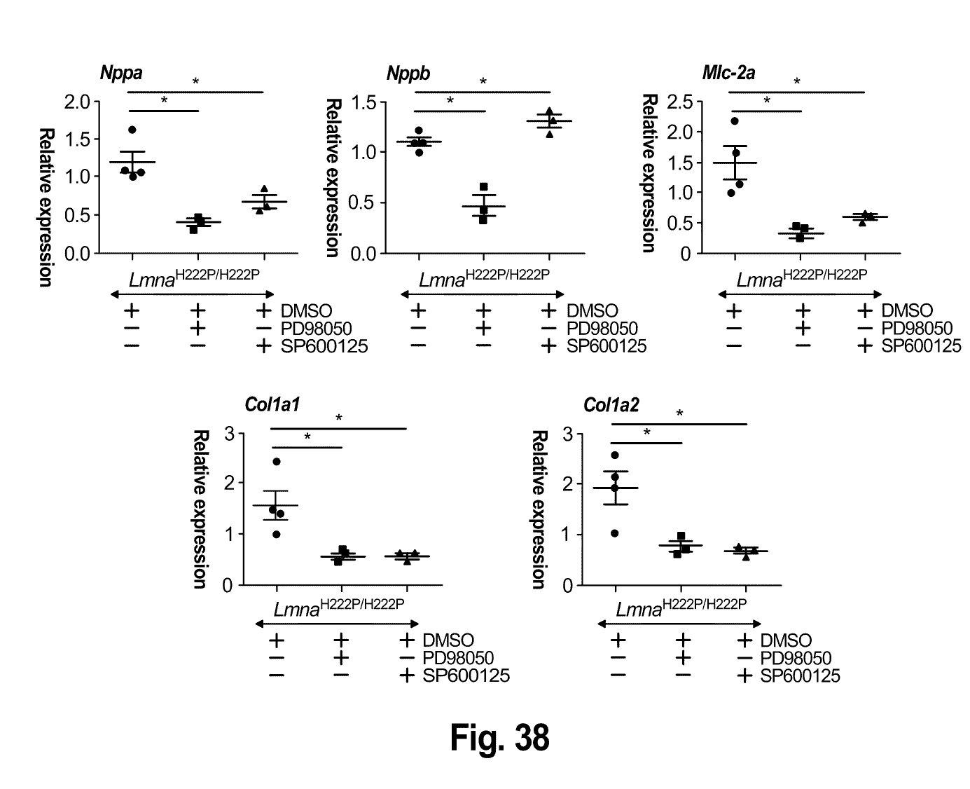

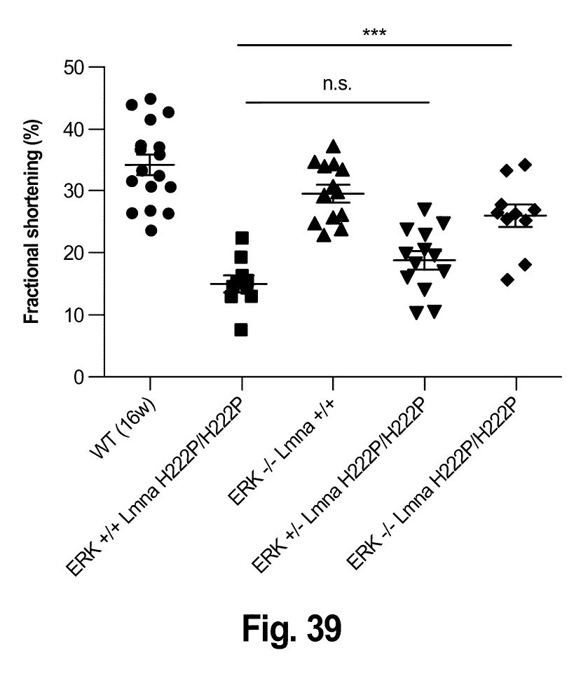

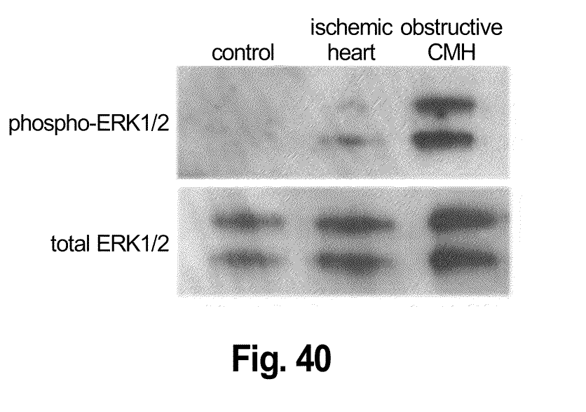

In a pilot study, we assessed treatment of Lmna^{H222P/H222P} mice with PD98059 and SP600125 at a more advanced stage of disease and for a longer time. We assigned male Lmna^{H222P/H222P} mice at 19 weeks of age to 3 different treatment arms (placebo DMSO, n=4; MEK1/2 inhibitor PD98059, n=3; JNK inhibitor SP600125, n=3) and examined parameters of cardiac structure and function. Systemic administration of PD98059 and SP600125 to Lmna^{H222P/H222P} mice partially blocked phosphorylation of ERK1/2 and JNK in hearts from 24 week-old mice (FIG. 37A). At 24 weeks, Lmna^{H222P/H222P} treated with PD98059 had decreased LV dilatation and increased FS compared to DMSO-treated mice (FIG. 37B). There was also a trend toward decreased LV dilatation and increased FS in the Lmna^{H222P/H222P} mice treated with SP600125 (FIG. 38). Cardiac expression of Mlc-2a, NppA, NppB, Colla1 and Colla2 mRNAs was also significantly reduced in the inhibitor-treated Lmna^{H222P/H222P} mice at 24 weeks, except for NppB in those treated with SP600125 (FIG. 38).

### Discussion

Our previous work has documented the effectiveness of inhibiting ERK and JNK signaling in preventing or delaying the onset of cardiomyopathy in Lmna^{H222P/H222P} mice (15,16). In those studies, MEK and JNK inhibitors were administered prior the onset of any detectable structural or functional cardiac abnormalities. A critical remaining question was if MEK and JNK inhibitors would be effective in improving heart function in Lmna^{H222P/H222P} mice when initiated after the onset of cardiac disease, which would be more analogous to treatment in human patients. In this study, we therefore tested the extent to which a treatment course starting after the onset of cardiac disease in Lmna^{H222P/H222P} mice would be beneficial.

Our results showed that pharmacological inhibitors of ERK and JNK signaling blocked increased expression of RNAs encoding natriuretic peptide precursors and proteins involved in sarcomere architecture, prevented LV end systolic dilatation, increased cardiac ejection fraction and decreased myocardial fibrosis. Two recent studies showed that either a calcium-sensitizing agent (25) or a β-blocker (24) also improved cardiac function in mouse models of Lmna-associated cardiomyopathy. This Example showed that MEK or JNK inhibitors could overcome the lack of definitive treatments for human patients suffering for cardiac disease caused by LMNA mutations.

Changes in myocardial structure and function in response to injury and proliferation of the non-myocyte cell populations of the heart, referred to as myocardial remodelling (26), alter cardiac performance over the long term. Part of such remodelling includes fibrosis, which results in exaggerated mechanical stiffness and causes systolic dysfunction (27). Established therapies for heart failure may also drive a significant part of their benefit from actions on cardiac fibroblasts. A beneficial effect on cardiac fibrosis has been reported for angiotensin converting enzyme inhibitors (28-30), angiotensin receptor blockers (31,32), diuretics (33) and aldosterone antagonists (34-36). Treatment of Lmna^{H222P/H222P} mice with MEK or JNK inhibitors had a profound beneficial effect on myocardial fibrosis, a characteristic of later-staged cardiomyopathy caused by LMNA mutations (21,22). Activation of ERK and JNK signaling pathways by various stimuli have been correlated to several cellular processes such as cell proliferation and remodelling of extra-cellular matrix (37) Inhibition of ERK and JNK signaling pathways had a beneficial effect on cardiac function by also acting directly to decrease the proliferation of myocardial fibroblasts.

Our study in Lmna^{H222P/H222P} mice was designed similar to a human clinical trial. It assessed primary endpoints (LV dilatation, EF) and “surrogate” secondary endpoints (expression of natriuretic peptide precursors) that are used in many human clinical heart failure trials. While mortality is a reasonable endpoint in phase III clinical trial for advanced heart failure, it is rarely if ever used in the initial drug assessment phase or in treatment of subjects with heart disease that is not end stage (38), as were both the case in our study. Furthermore, Lmna^{H222P/H222P} mice have diaphragmatic muscle involvement (not reported in humans with LMNA mutations) and significant skeletal muscle pathology as they age, which may be non-cardiac causes of mortality (13).

Nonetheless, the measurements of LV function we used correlate with prognosis in many human clinical trials and their behaviour parallels changes in mortality with treatment (38). For example, LV end-systolic volume, which is determined by measuring LVESD, is the major determinant of survival in human subjects after recovery from myocardial infarction and after coronary artery bypass grafting for impaired LV function (39,40). A study by Heywood et al. (41) also showed in human subjects with an EF less than 40% treated with angiotensin-converting enzyme inhibitors or angiotensin-receptor blockers that an increase of more than 15% in EF resulted in mortality of only about 2% per year. In our study, PD98059 and SP600125 improved the EF of Lmna^{H222P/H222P} mice approximately 22% and 15%, respectively, compared to placebo.

Taking into account that EF improvement is an important predictor for survival in human subjects with systolic dysfunction, small molecules inhibitors of the ERK and JNK signaling pathways could have a positive effect on survival of patients with LMNA mutations. While not an endpoint or our study, during the 4-week treatment protocol starting 16 weeks of age, 6 mice in the DMSO group, 3 in the PD98059 group and 3 in the SP600125 group died prior to reaching 20 weeks of age, suggesting that treatment with MEK1/2 or JNK inhibitors trended towards improved survival. Furthermore, our pilot study treating Lmna^{H222P/H222P} mice up to 24 weeks of age, when they have a mortality rate of approximately 25% (13), showed improvements in echocardiographic and cardiac biochemical parameters.

The choice of therapeutic agents in clinical trials is predicated, at least in part, on the efficacy of drugs studied in murine models of disease (42-44). For example, a second-generation oral MEK inhibitor, PD0325901 (Pfizer), has good potency against MEK, better bioavailability, increased metabolic stability and a longer time of MEK suppression (46). PD0325901 has been administered to humans and has entered a phase II clinical trial to treat advanced non-small cell lung cancer (47,48). Similarly, AZD6244/ARRY-142886 (AstraZeneca/Array Biopharma) is in phase II clinical trials for patients with cancers (49). Superior JNK inhibitors are also in preclinical development for use in humans (50). Hence, Lmna^{H222P/H222P} mice with cardiac dysfunction demonstrated the potential for clinical trials of MEK and JNK inhibitors, currently being developed for cancer and inflammatory conditions in human subjects with cardiomyopathy caused by LMNA mutations.

### REFERENCES FOR EXAMPLE 7

1. Taylor M R G, Carniel E, Mestroni L. Cardiomyopathy, familial dilated. Orphanet J Rare Dis. 2006; 13:1:27.
2. Worman H J, Fong L G, Muchir A, Young S G. Laminopathies and the long strange trip from basic cell biology to therapy. J Clin Invest. 2009; 119:1825-1836.
3. Bonne G, Di Barletta M R, Varnous S, Becane H M, Hammouda E H, Merlini L, Muntoni F, Greenberg C R, Gary F, Urtizberea J A, Duboc D, Fardeau M, Toniolo D, Schwartz K. Mutations in the gene encoding lamin A/C cause autosomal dominant Emery-Dreifuss muscular dystrophy. Nat Genet. 1999; 21:285-288.
4. Muchir A, Bonne G, van der Kooi A J, van Meegen M, Baas F, Bolhuis P A, de Visser M, Schwartz K. Identification of mutations in the gene encoding lamins A/C in autosomal dominant limb girdle muscular dystrophy with atrioventricular conduction disturbances (LGMD1B). Hum Mol Genet. 2000; 9:1453-1459.
5. Fatkin D, MacRae C, Sasaki T, Wolff M R, Porcu M, Frenneaux M, Atherton J, Vidaillet H J Jr, Spudich S, De Girolami U, Seidman J G, Seidman C, Muntoni F, Müehle G, Johnson W, McDonough B. Missense mutations in the rod domain of the lamin A/C gene as causes of dilated cardiomyopathy and conduction-system disease. N Engl J Med. 1999; 341:1715-1724.
6. Brodsky, G L, Muntoni, F, Miocic, S, Sinagra G, Sewry C, Mestroni L. Lamin A/C gene mutation associated with dilated cardiomyopathy with variable skeletal muscle involvement. Circulation. 2000; 101:473-476.
7. Taylor M R, Fain P R, Sinagra G, Robinson M L, Robertson A D, Carniel E, Di Lenarda A, Bohlmeyer T J, Ferguson D A, Brodsky G L, Boucek M M, Lascor J, Moss A C, Li W L, Stetler G L, Muntoni F, Bristow M R, Mestroni L, Familial Dilated Cardiomyopathy Registry Research Group. Natural history of dilated cardiomyopathy due to lamin A/C gene mutations. J Am Coll Cardiol. 2003; 41:771-780.
8. Vytopil M, Benedetti S, Ricci E, Galluzzi G, Dello Russo A, Merlini L, Boriani G, Gallina M, Morandi L, Politano L, Moggio M, Chiveri L, Hausmanova-Petrusewicz I, Ricotti R, Vohanka S, Toman J, Toniolo D. Mutation analysis of the lamin A/C gene (LMNA) among patients with different cardiomuscular phenotypes. J Med Genet. 2003; 40:e132.
9. van Tintelen J P, Hofstra R M, Katerberg H, Rossenbacker T, Wiesfeld A C, du Marchie Sarvaas G J, Wilde A A, van Langen I M, Nannenberg E A, van der Kooi A J, Kraak M, van Gelder I C, van Veldhuisen D J, Vos Y, van den Berg M P. High yield of LMNA mutations in patients with dilated cardiomyopathy and/or conduction disease referred to cardiogenetics outpatient clinics. Am Heart J. 2007; 154:1130-1139.
10. Cowan J, Li D, Gonzalez-Quintana J, Morales A, Hershberger R E. Morphological analysis of 13 LMNA variants identified in a cohort of 324 unrelated patients with idiopathic or familial dilated cardiomyopathy. Circ Cardiovasc Genet. 2010; 3:6-14.
11. van Berlo J H, de Voogt W G, van der Kooi A J, van Tintelen J P, Bonne G, Yaou R B, Duboc D, Rossenbacker T, Heidbilchel H, de Visser M, Crijns H J, Pinto Y M. Meta-analysis of clinical characteristics of 299 carriers of LMNA gene mutations: do lamin A/C mutations portend a high risk of sudden death? J Mol Med. 2005; 83:79-83.
12. Pasotti M, Klersy C, Pilotto A, Marziliano N, Rapezzi C, Serio A, Mannarino S, Gambarin F, Favalli V, Grasso M, Agozzino M, Campana C, Gavazzi A, Febo O, Marini M, Landolina M, Mortara A, Piccolo G, Viganò M, Tavazzi L, Arbustini E. Long-term outcome and risk stratification in dilated cardiolaminopathies. J Am Coll Cardiol. 2008; 52:1250-1260.
13. Arimura T, Helbling-Leclerc A, Massart C, Varnous S, Niel F, Lacene E, Fromes Y, Toussaint M, Mura A M, Keller D I, Amthor H, Isnard R, Malisses M, Schwartz K, Bonne G. Mouse model carrying H222P-lmna mutation develops muscular dystrophy and dilated cardiomyopathy similar to human striated muscle laminopathies. Hum Mol Genet. 2005; 14:155-169.
14. Muchir A, Pavlidis P, Decostre V, Herron A J, Arimura T, Bonne G, Worman H J. Activation of MAPK pathway links LMNA mutations to cardiomyopathy in Emery-Dreifuss muscular dystrophy. J Clin Invest. 2007; 117:1282-1293.
15. Muchir A, Shan J, Bonne G, Lehnart S E, Worman H J. Inhibition of extracellular signal-regulate kinase signaling to prevent cardiomyopathy caused by mutation in the gene encoding A-type lamins. Hum Mol Genet. 2009; 18:241-247.
16. Wu W, Shan J, Bonne G, Worman H J, Muchir A. Pharmacological inhibition of c-Jun N-terminal kinase prevents cardiomyopathy caused by mutation in LMNA gene. Biochim Biophys Acta. 2010; 1802:632-638.
17. Yoshimine K, Horiuchi M, Suzuki S, Kobayashi K, Abdul J M, Masuda M, Tomomura M, Ogawa Y, Itoh H, Nakao K, Osame M, Saheki T. Altered expression of atrial natriuretic peptide and contractile protein genes in hypertrophied ventricles of JVS mice with systemic carnitine deficiency. J Mol Cell Cardiol. 1997; 29:571-578.
18. Takahashi T, Allen P D, Izumo S. Expression of A-, B-, and C-type natriuretic peptide genes in failing and developing human ventricles. Correlation with expression of the Ca(2+)-ATPase gene. Circ Res. 1992; 71:9-17.
19. Hwang J J, Allen P D, Tseng G C, Lam C W, Fananapazir L, Dzau V J, Liew C C. Microarray gene expression profiles in dilated and hypertrophic cardiomyopathic end-stage heart failure. Physiol Genomics. 2002; 10:31-44.
20. Yung C K, Halperin V L, Tomaselli G F, Winslow R L. Gene expression profiles in end-stage human idiopathic dilated cardiomyopathy: altered expression of apoptotic and cytoskeletal genes. Genomics. 2004; 83:281-297.
21. Van Tintelen P J, Tio R A, Kerstjens-Frederikse W S, van Berlo J H, Boven L G, Suurmeijer A J H, White S J, den Dunnen J T, to Meerman G J, Vos Y J, van der Hout A H, Osinga J, van den Berg M P, van Verhuisen D J, Buys C H C M, Hofstra R M W, Pinto Y M. Severe myocardial fibrosis caused by a deletion of the 5′ end of the lamin A/C gene. J Am Coll Cardiol. 2007; 49:2430-2439.
22. Raman S V, Sparks E A, Baker P M, McCarthy B, Wooley C F. Mid-myocardial fibrosis by cardiac magnetic resonance in patients with lamin A/C cardiomyopathy: possible substrate for diastolic dysfunction. J Cardiovac Magn Res. 2007; 9:907-913.
23. Nikolova V, Leimena C, McMahon A C, Tan J C, CHandar S, Jogia D, Kesteven S H, Michalicek J, Otway R, Verheyen F, Rainer S, Stewart C L, Martin D, Feneley M P, Fatkin D. Defects in nuclear structure and function promote dilated cardiomyopathy in lamin A/C-deficient mice. J Clin Invest. 2004; 113:357-369.
24. Chandar S, Yeo L Z, Leimena C, Tan J C, Xiao X H, Nikolova-Krstevski V, Yasuoka Y, Gardiner-Garden M, Wu J, Kesteven S, Karlsdotter L, Natarajan S, Carlton A, Rainer S, Feneley M P, Fatkin D. Effects of mechanical stress and cardvedilol in lamin A/C-deficient dilated cardiomyopathy. Circ Res. 2010; 106:573-582.
25. Arimura T, Sato R, Machida N, Bando H, Zhan D Y, Morimoto S, Tanaka R, Yamane Y, Bonne G, Kimura A. Improvement of left ventricular dysfunction and of survival prognosis of dilated cardiomyopathy by administration of calcium sensitizer SCH00013 in a mouse model. J Am Coll Cardiol. 2010; 55:1502-1508.
26. Swynghedauw B. Molecular mechanisms of myocardial remodelling. Physiol Rev. 1999; 79:215-262.
27. Brown R D, Ambler S K, Mitchell M D, Long C S. The cardiac fibroblasts: therapeutic target in myocardial remodelling and failure. Annu Rev Pharmacol Toxicol. 2005; 45:657-687.
28. Sleight P. Angiotensin II and trials of cardiovascular outcomes. Am J Cardiol. 2002; 89:11-16.
29. Fox K M. Efficacy of perindopril in reduction of cardiovascular events among patients with stable coronary artery disease: randomised, double-blind, placebo-controlled, multicentre trial (the EUROPE study). Lancet. 2003; 362:782-788.
30. Brilla C G, Funck R C, Rupp H. Lisinopril-mediated regression of myocardial fibrosis in patients with hypertensive heart disease. Circulation. 2000; 102:1388-1393.
31. Pfeffer M A, Swedberg K, Granger C B, Held P, McMurray J J, Michelson E L, Olofsson B, Ostergen J, Yusuf S, Pocock S. Effects of candesartan on mortality and morbidity in patients with chronic heart failure: the CHARM-Overall programme Lancet. 2003; 362:759-766.
32. Diez J, Querej eta R, Lopez B, Gonzalez A, Larman M, Martinez Ubago J L. Losartan-dependent regression of myocardial fibrosis is associated with reduction of left ventricular chamber stiffness in hypertensive patients. Circulation. 2002; 105:2512-2517.
33. Lopez B, Gonzalez A, Beaumont J, Querejeta R, Larman M, Diez J. Identification of a potential cardiac antifibrotic mechanism of torasemide in patients with chronic heart failure. J Am Coll Cardiol. 2007; 50:859-867.
34. Pitt B, Zannad F, Remme W J, Cody R, Castaigne A, Perez A, Palensky J, Wittes J. The effect of spironolactone on morbidity and mortality in patients with severe heart failure. Randomized aldactone evaluation study investigators. N Engl J Med. 1999; 341:709-717.
35. Pitt B, Remme W, Zammad F, Neaton J, Martinez F, Roniker B, Bittman R, Hurley S, Kleiman J, Gatlin M. Eplerenone, a selective aldosterone blocker, in patients with left ventricular dysfunction after myocardial infarction. N Engl J Med. 2003; 348:1309-1321.
36. Zannad F, Alla F, Dousset B, Perez A, Pitt B. Limitation of excessive extracellular matrix turnover may contribute to survival benefit of spironolactone therapy in patients with congestive heart failure: insights from the randomized aldactone evaluation study (RALES). Circulation. 2000; 102:2700-2706.
37. Yoon S, Seger R. The extracellular signal-regulated kinase: Multiple substrates regulate diverse cellular functions. Growth Factors. 2006; 24:21-44.
38. Zanolla L, Zardini P. Selection of endpoints for heart failure clinical trials, Eur J Heart Fail. 2003; 5:717-723.
39. White H D, Norris, R M Brown M A, Brandt P W, Whitlock R M, Wild C J. Left ventricular end-systolic volume as the major determinant of survival after recovery from myocardial infarction, Circulation. 1987; 76:44-51.
40. Hamer A W, Takayama M, Abraham K A, Roche A H, Kerr A R, Williams B F, Ramage M C, White H D. End-systolic volume and long-term survival after coronary artery bypass graft surgery in patients with impaired left ventricular function, Circulation. 1994; 90:2899-2904.
41. Heywood J T, Elatre W, Pai R G, Fabbri S, Huiskes B. Simple Clinical Criteria to determine the prognosis of heart failure. J Cardiovasc Pharmacol Therapeut. 2005; 10:173-180.
42. Ikeda Y, Ross J. Models of dilated cardiomyopathy in the mouse and the hamster. Curr Opin Cardiol. 2000; 15:197-201.
43. Khurana T S, Davies K E. Pharmacological strategies for muscular dystrophy. Nat Rev Drug Discov. 2003; 2:379-390.
44. Bhatnagar S, Kumar A. Therapeutic targeting of signalling pathways in muscular dystrophy. J Mol Med. 2010; 88:155-166.
45. Allen L F, Sebolt-Leopold J, Meyer M B. C1-1040 (PD184352), a targeted signal transduction inhibitor f MEK (MAPKK). Semin Oncol. 2003; 30:105-116.
46. Brown A P, Carlson T C G, Loi C M, Graziano M J. Pharmacodynamic and toxicokinetic evaluation of the novel MEK inhibitor, PD0325901, in the rat following oral and intravenous administration. Cancer Chemother Pharmacol. 2007; 59:671-679.
47. Lorusso P, Krishnamurthi S, Rinehart J R, Nabell L, Croghan G, Varterasian M, Sadis S S, Menon S, Leopold J, Meyer M B. A phase 1-2 clinical study of a second generation oral MEK inhibitor, PD0325901 in patients with advanced cancer. J Clin Oncol. (Abstract) 2005; 23:3011.
48. Menon S S, Whitfield L R, Sadis S, Meyer M B, Leopold J, Lorusso P M, Krishnamurthi S, Rinehart J R, Nabell L, Croghan G. Pharmacokinetics (PK) and pharmacodynamics (PD) of PD0325901, a second generation MEK inhibitor after multiple oral doses of PD0325901 to advanced cancer patients. J Clin Oncol. (Abstract) 2005; 23:3066.
49. Adjei A A, Cohen R B, Franklin W, Morris C, Wilson D, Molina J R, Hanson L J, Gore L, Chow L, Leong S, Maloney L, Gordon G, Simmons H, Marlow A, Litwiler K, Brown S, Poch G, Kane K, Haney J, Eckhardt S G. Phase I pharmacokinetic and pharmacodynamic study of the oral, small-molecule mitogen-activated protein kinase kinase ½ inhibitor AZD6244 (ARRY-142886) in patients with advanced cancers. J Clin Oncol. 2008; 26:2139-2146.
50. Bogoyevitch M A, Ngoei K R, Zhao T T, Yeap Y Y, Ng D C. c-Jun N-terminal kinase (JNK) signaling: recent advances and challenges. Biochim Biophys Acta. 2010; 3:463-475.

We claim:

1. A method of treating or preventing a cardiomyopathy associated with activation of at least one kinase in the mitogen-activated protein kinase (MAPK) signaling pathway in heart tissue, the method comprising providing to a subject an inhibitor of at least one kinase in the extracellular signal-regulated kinase (ERK) signaling pathway, or an inhibitor of at least one kinase in the c-Jun N-terminal kinase (JNK) signaling pathway, or both.

2. The method of claim 1, wherein the cardiomyopathy is associated with one or more mutations in LMNA or EMD.

3. The method of claim 1, wherein the at least one kinase in the ERK signaling pathway is a MAPK/ERK kinase (MEK).

4. The method of claim 1, wherein the at least one kinase in the ERK signaling pathway is MEK1 or MEK2.

5. The method of claim 1, wherein the at least one kinase in the JNK signaling pathway is a JNK.

6. The method of claim 1, wherein the inhibitor of at least one kinase in the ERK signaling pathway is selected from the group consisting of a chromone and a flavone.

7. The method of claim 1, wherein the inhibitor of at least one kinase in the ERK signaling pathway is selected from the group consisting of 2-(2-amino-3-methoxyphenyl)-4H-1-benzopyran-4-one (PD98059), 1,4-diamino-2,3-dicyano-1,4-bis(2-aminophenylthio)butadiene (U0126), and Z-& E-a-(amino-((4-aminophenyl)thio)methylene)-2-(trifluoromethyl)benzeneacetonitrile (MEK1/2).

8. The method of claim 7, wherein the inhibitor of at least one kinase in the ERK signaling pathway is PD98059.

9. The method of claim 1, wherein the inhibitor of at least one kinase in the ERK signaling pathway is selected from the group consisting of PD0325901, AZD6244/ARRY-142886, and ARRY-438162.

10. The method of claim 1, wherein the inhibitor of at least one kinase in the JNK signaling pathway is an anthrapyrazolone.

11. The method of claim 10, wherein the inhibitor of at least one kinase in the JNK signaling pathway is anthra[1,9-cd]pyrazol-6(2H)-one (SP600125).

12. The method of claim 10, wherein the inhibitor of at least one kinase in the JNK signaling pathway is CC-401.

13. The method of claim 1, wherein the cardiomyopathy is dilated cardiomyopathy or a hypertrophic cardiomyopathy.

14. The method of claim 1, wherein the treating comprises improving cardiac function or preventing deterioration in cardiac function.

15. The method of claim 14, wherein the improving or preventing deterioration comprises increasing at least one of ejection fraction or fractional shortening.

16. The method of claim 14, wherein the improving or preventing deterioration comprises decreasing at least one of left ventricular end systolic diameter or left ventricular end diastolic diameter.

17. The method of claim 1, wherein the treating or the preventing comprises reducing expression of at least one molecular marker of cardiomyopathy.

18. The method of claim 17, wherein the molecular marker is selected from the group consisting of atrial natriuretic factor, brain natriuretic factor, Bcl-2, Elk-1, c-Jun, JunD, Vegf, Myl7, Sln, and Elk 4.

19. The method of claim 17, wherein the molecular marker is a sarcomere structure protein.

20. The method of claim 19, wherein the sarcomere structure protein is myosin.


SEQUENCE LISTING (17 sequences)

SEQ ID NO: 1 - protein, 13 aa, Unknown
  Description of Unknown MEK1 peptide
MPKKKPTPIQLNP

SEQ ID NO: 2 - DNA, 20 bp, Artificial Sequence
  Description of Artificial Sequence Synthetic oligonucleotide
cagccatcacctctcctttg

SEQ ID NO: 3 - DNA, 20 bp, Artificial Sequence
  Description of Artificial Sequence Synthetic oligonucleotide
agcaccagggagaggacagg

SEQ ID NO: 4 - DNA, 20 bp, Artificial Sequence
  Description of Artificial Sequence Synthetic primer
gcttccaggccatattggag

SEQ ID NO: 5 - DNA, 20 bp, Artificial Sequence
  Description of Artificial Sequence Synthetic primer
ccctgcttcctcagtctgct

SEQ ID NO: 6 - DNA, 20 bp, Artificial Sequence
  Description of Artificial Sequence Synthetic primer
ggaccaaggcctcacaaaag

SEQ ID NO: 7 - DNA, 20 bp, Artificial Sequence
  Description of Artificial Sequence Synthetic primer
tacagcccaaacgactgacg

SEQ ID NO: 8 - DNA, 20 bp, Artificial Sequence
  Description of Artificial Sequence Synthetic primer
tcaaggaagccttcagctgc

SEQ ID NO: 9 - DNA, 19 bp, Artificial Sequence
  Description of Artificial Sequence Synthetic primer
cggaacacttaccctcccg

SEQ ID NO: 10 - DNA, 20 bp, Artificial Sequence
  Description of Artificial Sequence Synthetic primer
agacggacagtactggatcg

SEQ ID NO: 11 - DNA, 20 bp, Artificial Sequence
  Description of Artificial Sequence Synthetic primer
gcttcttttccttggggttc

SEQ ID NO: 12 - DNA, 20 bp, Artificial Sequence
  Description of Artificial Sequence Synthetic primer
ccgtgcttctcagaacatca

SEQ ID NO: 13 - DNA, 20 bp, Artificial Sequence
  Description of Artificial Sequence Synthetic primer
gagcagccatcgactaggac

SEQ ID NO: 14 - DNA, 20 bp, Artificial Sequence
  Description of Artificial Sequence Synthetic primer
aatggaaaaggggaatggac

SEQ ID NO: 15 - DNA, 20 bp, Artificial Sequence
  Description of Artificial Sequence Synthetic primer
ctcggttgtccttcttgctc

SEQ ID NO: 16 - DNA, 19 bp, Artificial Sequence
  Description of Artificial Sequence Synthetic primer
tgcaccaccaactgcttag

SEQ ID NO: 17 - DNA, 19 bp, Artificial Sequence
  Description of Artificial Sequence Synthetic primer
ggatgcagggatgatgttc